(19) United States
(12) Patent Application Publication
(10) Pub. No.: US 2024/0228510 A1
(43) Pub. Date: Jul. 11, 2024

(54) KRAS G12C INHIBITORS

(71) Applicant: Mirati Therapeutics, Inc., San Diego, CA (US)

(72) Inventors: Aaron Craig Burns, San Diego, CA (US); James Gail Christensen, San Diego, CA (US); John David Lawson, Carlsbad, CA (US); Matthew Arnold Marx, San Diego, CA (US); Christopher Ronald Smith, San Diego, CA (US)

(21) Appl. No.: 18/286,961

(22) PCT Filed: Apr. 14, 2022
(86) PCT No.: PCT/US2022/024807

§ 371 Date: Oct. 13, 2023

Related U.S. Application Data
(60) Provisional application No. 63/175,891, filed on Apr. 16, 2021.

(51) Int. Cl. C07D 519/00; A61K 31/437; A61K 31/4375; A61K 31/438; A61K 31/496; C07D 471/04

(52) U.S. Cl. CPC C07D 519/00; A61K 31/437; A61K 31/4375; A61K 31/438; A61K 31/496; C07D 471/04

(57) ABSTRACT

The present invention relates to compounds that inhibit KRas G12C; in particular, the present invention relates to compounds that irreversibly inhibit the activity of KRas G12C, pharmaceutical compositions comprising the compounds and methods of use therefor.

### FIELD OF THE INVENTION

The present invention relates to compounds that inhibit KRas G12C. In particular, the present invention relates to compounds that irreversibly inhibit the activity of KRas G12C, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (e.g., see Dogan et al., (2012) Clin Cancer Res. 18(22):6169-6177, published online 2012 Sep. 26. doi: 10.1158/1078-0432.CCR-11-3265).

The well-known role of KRas in malignancy and the discovery of these frequent mutations in KRas in various tumor types made KRas a highly attractable target of the pharmaceutical industry for cancer therapy.

Despite many failed efforts to target KRas, compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well target KRas G12C (e.g., see Ostrem et al., (2013) Nature 503:548-551). Clearly there remains a continued interest and effort to develop inhibitors of KRas, particularly inhibitors of activating KRas mutants, including KRas G12C.

Thus, there is a need to develop new KRas G12C inhibitors that demonstrate sufficient efficacy, stability and/or safety for treating KRas G12C-mediated cancer. The compounds and compositions of the present invention advantageously overcome one or more of the previous shortcomings by providing selective KRas G12C inhibitors.

### SUMMARY OF THE INVENTION

In one aspect of the invention, compounds are provided that inhibit KRas G12C activity. In certain embodiments, the compounds are represented by Formula (I):

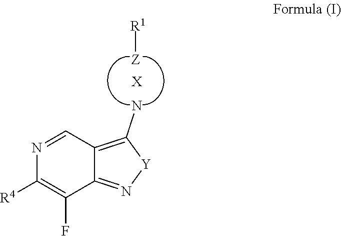

or a pharmaceutically acceptable salt thereof, wherein:
X is a 4-12 membered saturated or partially saturated monocyclic, bridged, spirocyclic or fused bicyclic heterocyclic ring system, wherein said heterocyclic ring system is optionally substituted with one or more R⁵;
Y is C(R²)═C(R³) or S;
Z is N, C(H)—N(H)— or C(H)—N(CH₃)—;
R¹ is —C(O)C(R^{A}) [symbol] C(R^{B})ₚ, where
   p is 1 or 2,
   R^{A} is absent, hydrogen, deuterium, cyano, halogen, C1-C6 alkyl, halo-C1-C6 alkyl, heteroalkyl or hydroxy-C1-C6 alkyl, and
   each R^{B} is independently hydrogen, deuterium, cyano, C1-C6 alkyl, alkoxy, halogen or halo-C1-C6 alkyl;
R² is absent, hydrogen, C1-C6 alkyl, alkoxy, halogen, cyano or C2-C6 alkynyl;
R³ is absent, hydrogen, C1-C6 alkyl, alkoxy, halogen, cyano or C2-C6 alkynyl;
R⁴ is 3-12 member heterocyclyl, 3-12 member cycloalkyl, C6-C14 aryl, C6-C14 aryl-C1-C6 alkyl or 5-14 member heteroaryl, wherein R⁴ is optionally substituted with one or more substituents independently selected from C1-C6 alkyl, C2-C6 alkenyl, C2-C6 alkynyl, 3-12 member cycloalkyl, amino, amino-C1-C6alkyl, hydroxy, alkoxy, halogen, cyano, and C1-C6 alkylamino; and
R⁵ is C1-C6 alkyl, cyano, C1-C6 alkyl-cyano, halogen, alkoxy, hydroxy, amino, C1-C6 alkylamino.

Also included are compounds of Formula (I) having the Formula (IA):

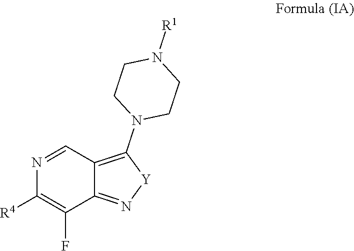

where R¹, R⁴ and Y are as defined for Formula (I).

Also included are compounds of Formula (I) having the Formula (IB):

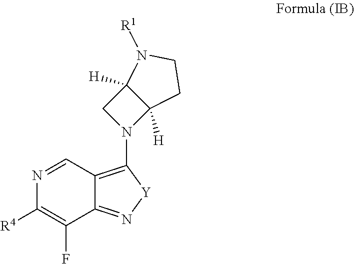

where R¹, R⁴ and Y are as defined for Formula (I), and where the fused bicylic heterocyclic ring system is optionally substituted with an alkyl, cyanoalkyl or halogen.

In another aspect of the invention R⁴ is naphthyl, or is naphthyl and is substituted with fluoro and chloro, and is naphthyl and is substituted with cyano, or is naphthyl and is substituted with fluoro and cyano, or is naphthyl and is substituted with hydroxy and cyano.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting KRas G12C activity in a in a cell, comprising contacting the cell with a compound of Formula (I), Formula (IA) and Formula (IB). In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), Formula (IA) and Formula (IB), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a KRas G12C-associated disease or disorder in a patient in need of such treatment, the method comprising administering to the patient a therapeutically effective amount of a compound of Formula (I), Formula (IA) and Formula (IB), or a pharmaceutically acceptable salt or solvate thereof, or a pharmaceutical composition thereof as defined herein.

Also provided herein is a compound of Formula (I), Formula (IA) and Formula (IB), or a pharmaceutically acceptable salt or solvate thereof, or a pharmaceutical composition thereof as defined herein for use in therapy.

Also provided herein is a compound of Formula (I), Formula (IA) and Formula (IB), or a pharmaceutically acceptable salt or solvate thereof or a pharmaceutical composition thereof as defined herein for use in the treatment of cancer.

Also provided herein is a compound of Formula (I), Formula (IA) and Formula (IB), or a pharmaceutically acceptable salt or solvate thereof for use in the inhibition of KRas G12C.

Also provided herein is a compound of Formula (I), Formula (IA) and Formula (IB), or a pharmaceutically acceptable salt or solvate thereof or a pharmaceutical composition thereof as defined herein, for use in the treatment of a KRas G12C-associated disease or disorder.

Also provided herein is the use of a compound of Formula (I), Formula (IA) and Formula (IB), or a pharmaceutically acceptable salt or solvate thereof, as defined herein in the manufacture of a medicament for the treatment of cancer.

Also provided herein is a use of a compound of Formula (I), Formula (IA) and Formula (IB), or a pharmaceutically acceptable salt or solvate thereof, as defined herein in the manufacture of a medicament for the inhibition of activity of KRas G12C.

Also provided herein is the use of a compound of Formula (I), Formula (IA) and Formula (IB), or a pharmaceutically acceptable salt or solvate thereof, as defined herein, in the manufacture of a medicament for the treatment of a KRas G12C-associated disease or disorder.

Also provided herein is a method for treating cancer in a patient in need thereof, the method comprising (a) determining that the cancer is associated with a KRas G12C mutation (e.g., a KRas G12C-associated cancer); and (b) administering to the patient a therapeutically effective amount of a compound of Formula (I), Formula (IA) and Formula (IB), or a pharmaceutically acceptable salt thereof, or a pharmaceutical composition thereof.

Also provided herein is a process for preparing a compound of Formula (I), Formula (IA) and Formula (IB), or a pharmaceutically acceptable salt or solvate thereof.

Also provided herein is a compound of Formula (I), Formula (IA) and Formula (IB), or a pharmaceutically acceptable salt thereof obtained by a process of preparing the compound as defined herein.

### DETAILED DESCRIPTION OF THE INVENTION

The present invention relates to inhibitors of KRas G12C. In particular, the present invention relates to compounds that irreversibly inhibit the activity of KRas G12C, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms. Substituents can include any substituents described herein, for example, but not limited to, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

As used herein, a “KRas G12C inhibitor” refers to compounds of the present invention that are represented by formulae (I) as described herein. These compounds are capable of negatively modulating or inhibiting all or a portion of the enzymatic activity of KRas G12C. The KRas G12C inhibitors of the present invention interact with and irreversibly bind to KRas G12C by forming a covalent adduct with the sulfhydryl side chain of the cysteine residue at position 12 resulting in the inhibition of the enzymatic activity of KRas G12C.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12C mutation (e.g., as determined using a regulatory agency-approved, e.g., FDA-approved, assay or kit). In some embodiments, the subject has a tumor that is positive for a KRas G12C mutation (e.g., as determined using a regulatory agency-approved assay or kit). The subject can be a subject with a tumor(s) that is positive for a KRas G12C mutation (e.g., identified as positive using a regulatory agency-approved, e.g., FDA-approved, assay or kit). The subject can be a subject whose tumors have a KRas G12C mutation (e.g., where the tumor is identified as such using a regulatory agency-approved, e.g., FDA-approved, kit or assay). In some embodiments, the subject is suspected of having a KRas G12C gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has KRas G12C mutation using a sample (e.g., a biological sample or a biopsy sample (e.g., a paraffin-embedded biopsy sample) from a patient (e.g., a patient suspected of having a KRas G12C-associated cancer, a patient having one or more symptoms of a KRas G12C-associated cancer, and/or a patient that has an increased risk of developing a KRas G12C-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “amino” refers to —NH₂.

The term “acyl” refers to —C(O)CH₃.

The term “alkyl” as employed herein refers to straight and branched chain aliphatic groups having from 1 to 12 carbon atoms, 1-8 carbon atoms 1-6 carbon atoms, or 1-3 carbon atoms which is optionally substituted with one, two or three substituents. Examples of alkyl groups include, without limitation, methyl, ethyl, propyl, isopropyl, butyl, isobutyl, sec-butyl, tert-butyl, pentyl, and hexyl.

The term “haloalkyl” refers to an alkyl chain as defined herein above, in which one or more hydrogen has been replaced by a halogen. Examples of haloalkyls are trifluoromethyl, difluoromethyl and fluoromethyl.

The term “haloalkyloxy” refers to —O-haloalkyl.

An “alkylene,” group is an alkyl group, as defined hereinabove, that is positioned between and serves to connect two other chemical groups. Exemplary alkylene groups include, without limitation, methylene, ethylene, propylene, and butylene.

The term “alkoxy” refers to —OC1-C6 alkyl.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, and N.

As used herein, the term “hydroxyalkyl” refers to an alkyl chain, as defined herein above, wherein one hydrogen atom is replaced with a hydroxyl group.

The term “dihydroxyalkyl” refers to an alkyl group as defined herein wherein two carbon atoms are each substituted with a hydroxyl group.

The term “alkylaminyl” refers to —NR^{x}-alkyl, wherein R^{x} is hydrogen.

The term “dialkylaminyl” refers to —N(R^{y})₂, wherein each R^{y} is independently C1-C3 alkyl.

The term “alkylaminylalkyl” refers to -alkyl-NR^{x}-alkyl, wherein R^{x} is hydrogen.

The term “dialkylaminylalkyl” refers to -alkyl-N(R^{y})₂, wherein each R^{y} is independently C1-C4 alkyl, wherein the alkyl of the -alkyl-N(R^{y})₂ is an alkyl group as defined hereinabove and may be optionally substituted with hydroxy or hydroxyalkyl.

An “aryl” group is a C₆-C₁₄ aromatic moiety comprising one to three aromatic rings, which is optionally substituted. As one embodiment, the aryl group is a C₆-C₁₀ aryl group. Examples of aryl groups include, without limitation, phenyl, naphthyl, anthracenyl, fluorenyl, and dihydrobenzofuranyl. An “aryl” group may be optionally include one aromatic ring fused to a heterocyclyl.

An “aralkyl” or “arylalkyl” group comprises an aryl group covalently linked to an alkyl group as defined herein above, either of which may independently be optionally substituted or unsubstituted. An example of an aralkyl group is (C₁-C₆)alkyl(C₆-C₁₀)aryl, including, without limitation, benzyl, phenethyl, and naphthylmethyl. An example of a substituted aralkyl is wherein the alkyl group is substituted with hydroxyalkyl.

A “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. The heterocyclic group is optionally substituted with R⁷ on carbon or nitrogen at one or more positions, wherein R⁷ is as defined for Formula (I). The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, thiazolidinyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, hexahydrofuro[3.2-b]furanyl, (3R, 3aR, 6R, 6aR)-hydroxyhexahydrofuro[3.2-b]furanyl, morpholinyl, oxazepanyl, and azabicyclohexanes, azabicycloheptanes and oxa azabiocycloheptanes, including diazabicyclo[3.2.0]heptan-6-yl, diazabicyclo[3.2.0]heptan-2-yl, diazabicyclo[3.2.1]octan-8-yl or diazabicyclo[3.2.1]octan-3-yl. Specifically excluded from the scope of this term are compounds having adjacent annular O and/or S atoms.

The term “heterocyclylalkyl” refers to a heterocyclyl group as defined herein covalently linked to an alkyl group as defined hereinabove wherein the radical is on the alkyl group, wherein the alkyl group of the heterocyclylalkyl may be optionally substituted with hydroxy or hydroxyalkyl.

As used herein, the term “heteroaryl” refers to groups having 5 to 14 ring atoms, preferably 5, 6, 9, or 10 ring atoms; having 6, 10, or 14π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “heteroarylalkyl” group comprises a heteroaryl group covalently linked to an alkyl group, wherein the radical is on the alkyl group, either of which is independently optionally substituted or unsubstituted. Examples of heteroarylalkyl groups include a heteroaryl group having 5, 6, 9, or 10 ring atoms bonded to a C1-C6 alkyl group. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent annular O and/or S atoms.

As used herein, “an effective amount” of a compound is an amount that is sufficient to negatively modulate or inhibit the activity of KRas G12C. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of KRas G12C. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of the KRAS inhibitor detailed herein or a pharmaceutically acceptable salt thereof, or the length of treatment time described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### Compounds

In certain embodiments of the invention there are provided compounds Formula (I):

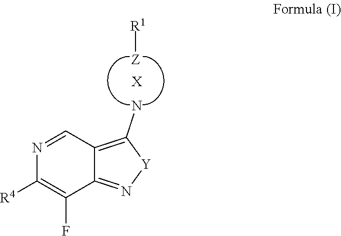

or a pharmaceutically acceptable salt thereof, wherein:
X is a 4-12 membered saturated or partially saturated monocyclic, bridged, spirocyclic or fused bicyclic heterocyclic ring system, wherein said heterocyclic ring system is optionally substituted with one or more R⁵;
Y is C(R²)═C(R³) or S;
Z is N, C(H)—N(H)— or C(H)—N(CH₃)—
R¹ is —C(O)C(R^{A}) [symbol] C(R^{B})ₚ, where
   p is 1 or 2,
   R^{A} is absent, hydrogen, deuterium, cyano, halogen, C1-C6 alkyl, halo-C1-C6 alkyl, heteroalkyl or hydroxy-C1-C6 alkyl, and
   each R^{B} is independently hydrogen, deuterium, cyano, C1-C6 alkyl, alkoxy, halogen or halo-C1-C6 alkyl;
R² is absent, hydrogen, C1-C6 alkyl, alkoxy, halogen, cyano or C2-C6 alkynyl;
R³ is absent, hydrogen, C1-C6 alkyl, alkoxy, halogen, cyano or C2-C6 alkynyl;
R⁴ is 3-12 member heterocyclyl, 3-12 member cycloalkyl, C6-C14 aryl, C6-C14 aryl-C1-C6 alkyl or 5-14 member heteroaryl, wherein R⁴ is optionally substituted with one or more substituents independently selected from C1-C6 alkyl, C2-C6 alkenyl, C2-C6 alkynyl, 3-12 member cycloalkyl, amino, amino-C1-C6 alkyl, hydroxy, alkoxy, halogen, cyano, and C1-C6 alkylamino; and
R⁵ is C1-C6 alkyl, cyano, C1-C6 alkyl-cyano, halogen, alkoxy, hydroxy, amino, C1-C6 alkylamino.

Embodiments of the invention also include compounds of Formula (I) having the Formula (IA):

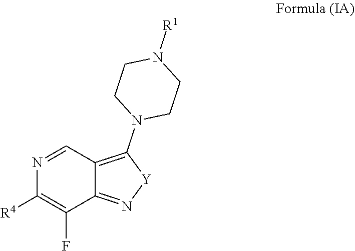

Formula (IA)
where R¹, R⁴ and Y are as defined for Formula (I).

Embodiments of the invention also include compounds of Formula (I) having the Formula (IB):

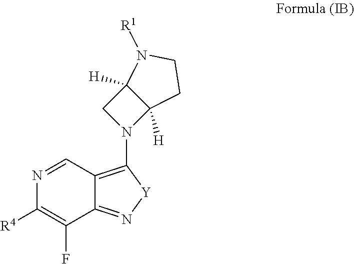

where R¹, R⁴ and Y are as defined for Formula (I), and the fused bicylic heterocyclic ring system is optionally substituted with an alkyl, cyanoalkyl or halogen.

Embodiments of the invention include compounds having the formula:

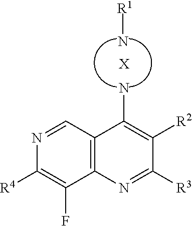

where X, R¹, R², R³ and R⁴ are as defined for Formula (I), or pharmaceutically acceptable salts thereof.

Embodiments of the invention further include compounds having the formula:

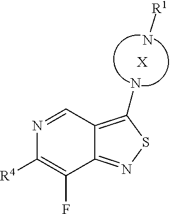

where X, R¹ and R⁴ are as defined for Formula (I), or pharmaceutically acceptable salts thereof.

Embodiments of the invention further include compounds having the formula:

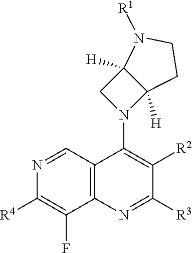

where R¹, R², R³ and R⁴ are as defined for Formula (I) or (IB), or pharmaceutically acceptable salts thereof.

Embodiments of the invention include compounds having the formula:

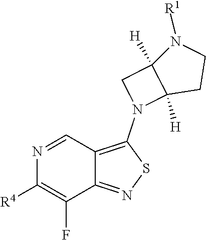

where R¹ and R⁴ are as defined for Formula (I), or pharmaceutically acceptable salts thereof.

Embodiments of the invention include compounds having the formula:

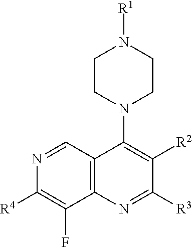

where R¹, R², R³ and R⁴ are as defined for Formula (I), or pharmaceutically acceptable salts thereof.

Embodiments of the invention further include compounds having the formula:

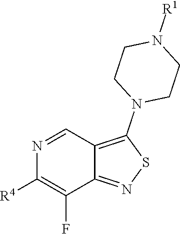

where R¹ and R⁴ are as defined for Formula (I), or pharmaceutically acceptable salts thereof.

In certain embodiments, R¹—X is:

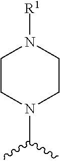

wherein R¹ is as defined for Formula (I).

In certain embodiments, R¹—X is:

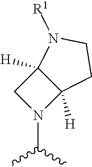

wherein R¹ is as defined for Formula (I), and the bicylic ring system is optionally substituted with an alkyl, cyanoalkyl or halogen.

In certain embodiments, R⁴ is naphthyl. In certain embodiments where R⁴ is naphthyl, naphthyl is substituted with fluoro and chloro. In certain embodiments where R⁴ is naphthyl, naphthyl is substituted with cyano. In certain embodiments where R⁴ is naphthyl, naphthyl is substituted with fluoro and cyano. In certain embodiments where R⁴ is naphthyl, naphthyl is substituted with hydroxy and cyano.

In one embodiment, the spirocyclic ring system is unsubstituted. Non-limiting examples of spirocyclic ring systems include:

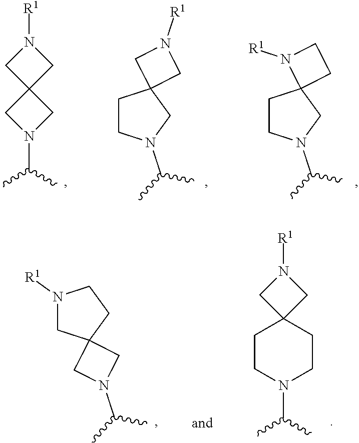

Nonlimiting examples of compounds of Formula (I), Formula (IA) and Formula (IB) are selected from the group consisting of:

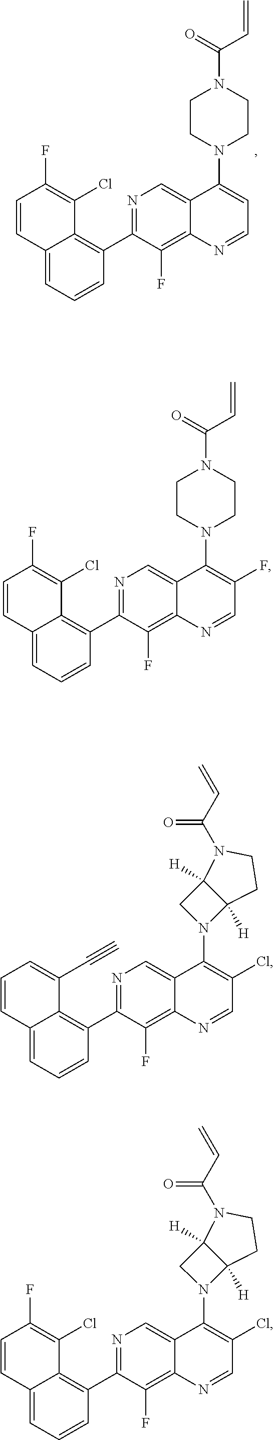 [SMILES: C=CC(=O)N1CCN(c2ccnc3c(F)c(-c4cccc5ccc(F)c(Cl)c45)ncc23)CC1 ; C=CC(=O)N1CCN(c2c(F)cnc3c(F)c(-c4cccc5ccc(F)c(Cl)c45)ncc23)CC1 ; C#Cc1cccc2cccc(-c3ncc4c(N5C[C@@H]6[C@H]5CCN6C(=O)C=C)c(Cl)cnc4c3F)c12 ; C=CC(=O)N1CC[C@@H]2[C@H]1CN2c1c(Cl)cnc2c(F)c(-c3cccc4ccc(F)c(Cl)c34)ncc12]

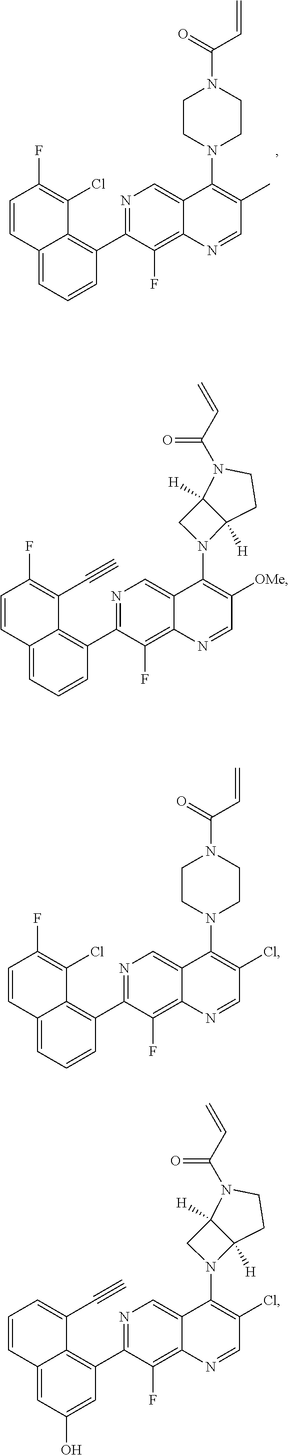 [SMILES: C=CC(=O)N1CCN(c2c(C)cnc3c(F)c(-c4cccc5ccc(F)c(Cl)c45)ncc23)CC1 ; C#Cc1c(F)ccc2cccc(-c3ncc4c(N5C[C@@H]6[C@H]5CCN6C(=O)C=C)c(OC)cnc4c3F)c12 ; C=CC(=O)N1CCN(c2c(Cl)cnc3c(F)c(-c4cccc5ccc(F)c(Cl)c45)ncc23)CC1 ; C#Cc1cccc2cc(O)cc(-c3ncc4c(N5C[C@@H]6[C@H]5CCN6C(=O)C=C)c(Cl)cnc4c3F)c12]

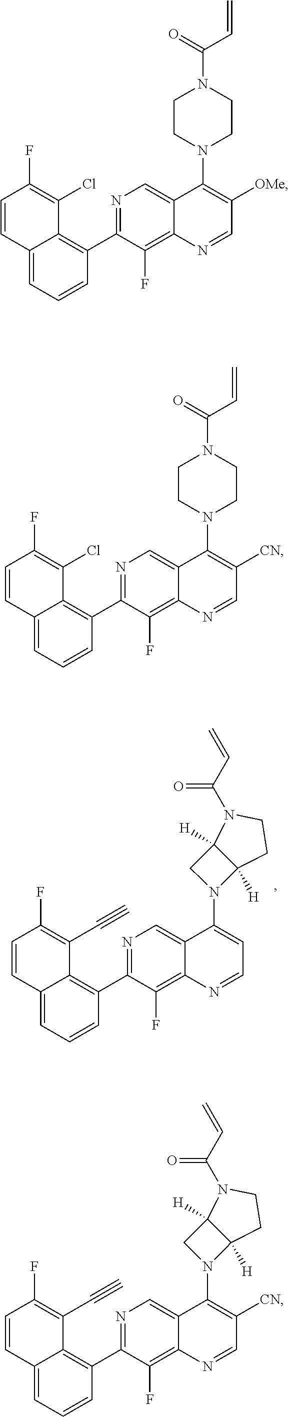 [SMILES: C=CC(=O)N1CCN(c2c(OC)cnc3c(F)c(-c4cccc5ccc(F)c(Cl)c45)ncc23)CC1 ; C=CC(=O)N1CCN(c2c(C#N)cnc3c(F)c(-c4cccc5ccc(F)c(Cl)c45)ncc23)CC1 ; C#Cc1c(F)ccc2cccc(-c3ncc4c(N5C[C@@H]6[C@H]5CCN6C(=O)C=C)ccnc4c3F)c12 ; C#Cc1c(F)ccc2cccc(-c3ncc4c(N5C[C@@H]6[C@H]5CCN6C(=O)C=C)c(C#N)cnc4c3F)c12]

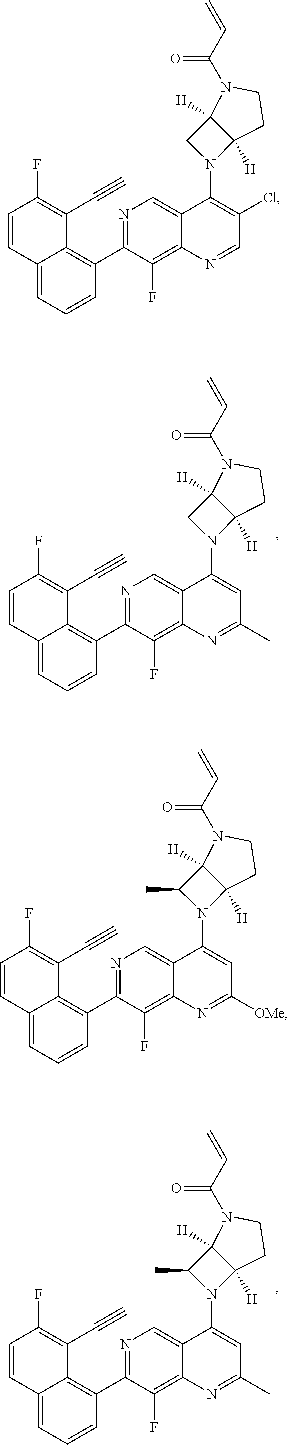 [SMILES: C#Cc1c(F)ccc2cccc(-c3ncc4c(N5C[C@@H]6[C@H]5CCN6C(=O)C=C)c(Cl)cnc4c3F)c12 ; C#Cc1c(F)ccc2cccc(-c3ncc4c(N5C[C@@H]6[C@H]5CCN6C(=O)C=C)cc(C)nc4c3F)c12 ; C#Cc1c(F)ccc2cccc(-c3ncc4c(N5[C@@H]6CCN(C(=O)C=C)[C@@H]6[C@@H]5C)cc(OC)nc4c3F)c12 ; C#Cc1c(F)ccc2cccc(-c3ncc4c(N5[C@@H]6CCN(C(=O)C=C)[C@@H]6[C@@H]5C)cc(C)nc4c3F)c12]

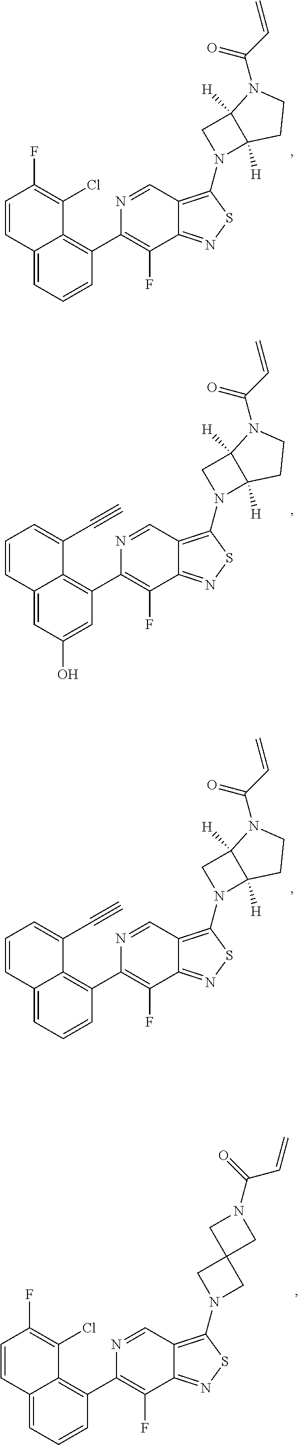 [SMILES: C=CC(=O)N1CC[C@@H]2[C@H]1CN2c1snc2c(F)c(-c3cccc4ccc(F)c(Cl)c34)ncc12 ; C#Cc1cccc2cc(O)cc(-c3ncc4c(N5C[C@@H]6[C@H]5CCN6C(=O)C=C)snc4c3F)c12 ; C#Cc1cccc2cccc(-c3ncc4c(N5C[C@@H]6[C@H]5CCN6C(=O)C=C)snc4c3F)c12 ; C=CC(=O)N1CC2(C1)CN(c1snc3c(F)c(-c4cccc5ccc(F)c(Cl)c45)ncc13)C2]

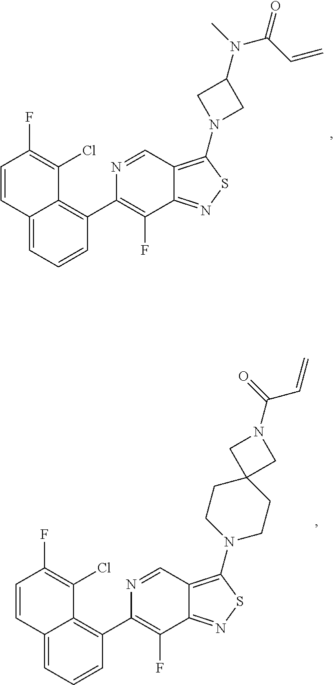 [SMILES: C=CC(=O)N(C)C1CN(c2snc3c(F)c(-c4cccc5ccc(F)c(Cl)c45)ncc23)C1 ; C=CC(=O)N1CC2(CCN(c3snc4c(F)c(-c5cccc6ccc(F)c(Cl)c56)ncc34)CC2)C1]

and pharmaceutically acceptable salts thereof.

### Pharmaceutical Compositions

The compounds of Formula (I) may be formulated into pharmaceutical compositions. Therefore, in another aspect, the invention provides pharmaceutical compositions comprising a KRas G12C inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting KRas G12C activity in a cell, comprising contacting the cell in which inhibition of KRas G12C activity is desired with an effective amount of a compound of Formula (I), Formula (IA) and Formula (IB), pharmaceutically acceptable salts thereof or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a KRas G12C with a compound provided herein includes the administration of a compound provided herein to an individual or patient, such as a human, having KRas G12C, as well as, for example, introducing a compound provided herein into a sample containing a cellular or purified preparation containing the KRas G12C.

In one embodiment, a cell in which inhibition of KRas G12C activity is desired is contacted with an effective amount of a compound of Formula (I), Formula (IA) and Formula (IB) to negatively modulate the activity of KRas G12C. In other embodiments, a therapeutically effective amount of pharmaceutically acceptable salt or pharmaceutical compositions containing the compound of Formula (I), Formula (IA) and Formula (IB), may be used.

By negatively modulating the activity of KRas G12C, the methods described herein are designed to inhibit undesired cellular proliferation resulting from enhanced KRas G12C activity within the cell. The cells may be contacted in a single dose or multiple doses in accordance with a particular treatment regimen to effect the desired negative modulation of KRas G12C. The inhibitory activity of exemplary compounds in cells may be monitored, for example, by measuring the inhibition of KRas G12C activity of the amount of phosphorylated ERK, including those described in Example A below, to assess the effectiveness of treatment and dosages may be adjusted accordingly by the attending medical practitioner.

In another aspect, methods of treating cancer in a patient in need thereof, comprising administering to said patient a therapeutically effective amount of a compound of Formula (I), Formula (IA) and Formula (IB), pharmaceutically acceptable salts thereof or pharmaceutical compositions comprising the compound or pharmaceutically acceptable salts thereof are provided.

The compositions and methods provided herein may be used for the treatment of a KRas G12C-associated cancer in a patient in need thereof, comprising administering to said patient a therapeutically effective amount of a compound of Formula (I), Formula (IA) and Formula (IB), pharmaceutically acceptable salts thereof or pharmaceutical compositions comprising the compound or pharmaceutically acceptable salts thereof are provided. In one embodiment, the KRas G12C-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (I), Formula (IA) and Formula (IB), or a pharmaceutically acceptable salt or solvate thereof, or a pharmaceutical composition thereof as defined herein for use in therapy.

Also provided herein is a compound of Formula (I), Formula (IA) and Formula (IB), or a pharmaceutically acceptable salt or solvate thereof or a pharmaceutical composition thereof as defined herein for use in the treatment of cancer.

Also provided herein is a compound of Formula (I), Formula (IA) and Formula (IB), or a pharmaceutically acceptable salt or solvate thereof for use in the inhibition of KRas G12C.

Also provided herein is a compound of Formula (I), Formula (IA) and Formula (IB), or a pharmaceutically acceptable salt or solvate thereof or a pharmaceutical composition thereof as defined herein, for use in the treatment of a KRas G12C-associated disease or disorder.

Also provided herein is the use of a compound of Formula (I), Formula (IA) and Formula (IB), or a pharmaceutically acceptable salt or solvate thereof, as defined herein in the manufacture of a medicament for the treatment of cancer.

Also provided herein is a use of a compound of Formula (I), Formula (IA) and Formula (IB), or a pharmaceutically acceptable salt or solvate thereof, as defined herein in the manufacture of a medicament for the inhibition of activity of KRas G12C.

Also provided herein is the use of a compound of Formula (I), Formula (IA) and Formula (IB), or a pharmaceutically acceptable salt or solvate thereof, as defined herein, in the manufacture of a medicament for the treatment of a KRas G12C-associated disease or disorder.

Also provided herein is a method for treating cancer in a patient in need thereof, the method comprising (a) determining that cancer is associated with a KRas G12C mutation (e.g., a KRas G12C-associated cancer) (e.g., as determined using a regulatory agency-approved, e.g., FDA-approved, assay or kit); and (b) administering to the patient a therapeutically effective amount of a compound of Formula (I), Formula (IA) and Formula (IB), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### REACTION SCHEMES AND EXAMPLES

The compounds of the present invention may be prepared from commercially available reagents using the synthetic methods and reaction schemes described herein, or using other reagents and conventional methods well known to those skilled in the art.

For instance, compounds of the present invention may be prepared according to the General Reaction Schemes I-III.

### General Reaction Schemes

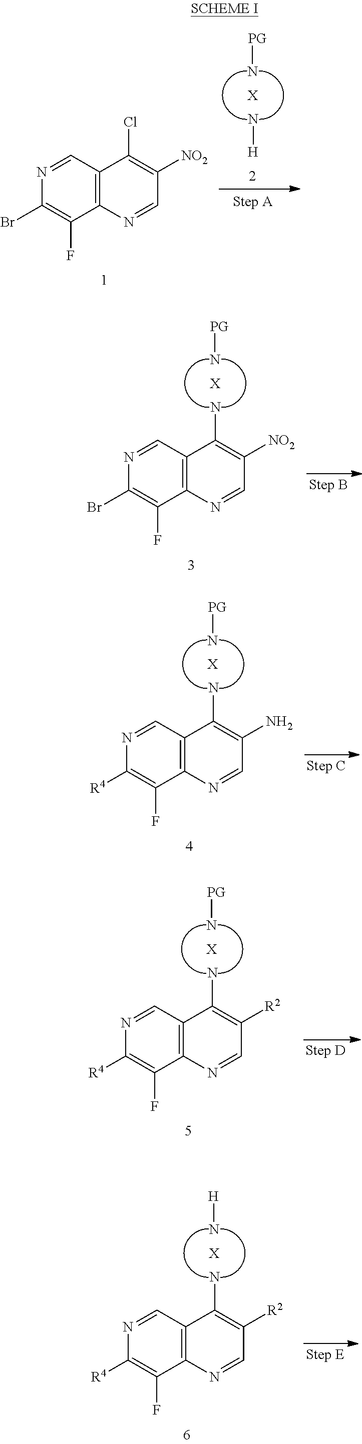

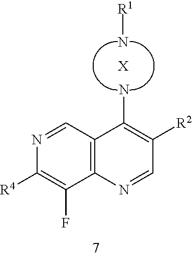

Compounds of Formula (I) where R² is a hydrogen, alkyl, halogen, or cyano can be prepared according to general Scheme I. A suitably substituted compound 1 is reacted in Step A with a heterocycle, wherein one of the nitrogen atoms is protected with a suitable nitrogen protecting group PG, such as a tert-butoxycarbonyl group. In Step B the nitro moiety is reduced and an R⁴ group is accomplished by using a suitably functionalized R⁴, for example a boronic acid or boronate ester, in the presence of a palladium catalyst and a base such as potassium phosphate in a solvent such as dioxane. In some instances the order of reduction and cross-coupling is reversed. In Step C, the amino group is converted into a halogen and in some cases then to the respective functional group (e.g., alkyl or cyano) using standard palladium catalyzed cross-coupling conditions. In Step E, the protecting group is removed under standard conditions. For example, if the protecting group is a tert-butoxycarbonyl group, it can be removed upon treatment with trifluoroacetic acid, in a solvent such as dichloromethane. The R¹ group is introduced, for example by treatment of intermediate 6 with an acid anhydride in the presence of a base such as diisopropylethylamine in a solvent such as dichloromethane.

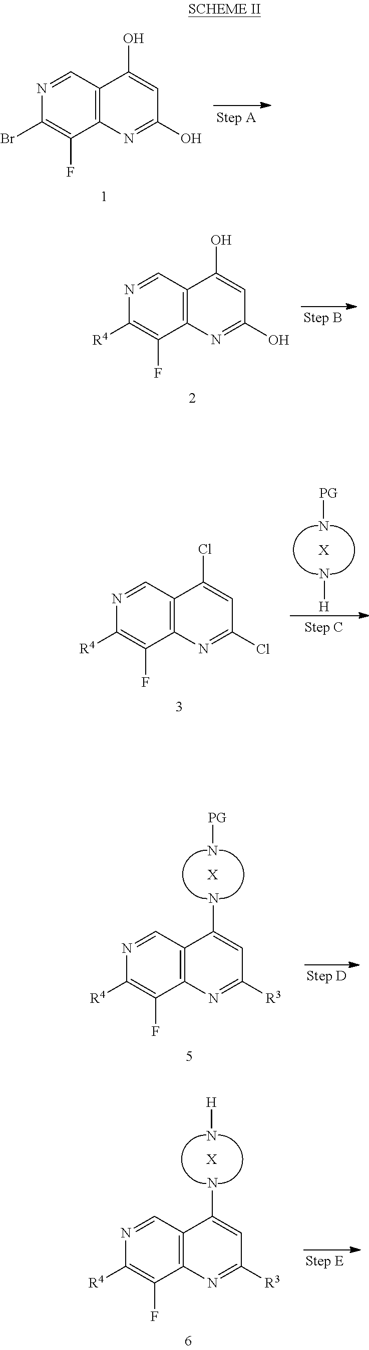

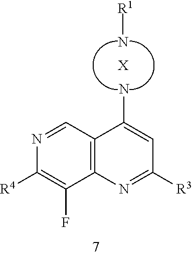

Compounds of Formula (I) where R³ is an alkoxy or alkyl substituent can be prepared according to general Scheme II. In Step A, coupling of an R⁴ group is accomplished by using a suitably functionalized R⁴, for example a boronic acid or boronate ester, in the presence of a palladium catalyst and a base such as potassium phosphate in a solvent such as dioxane. In Step B, the hydroxyl moieties are converted to suitable leaving groups, such as chlorine atoms. In Step C, one of the leaving groups in intermediate 3 is substituted with a heterocycle, wherein one of the nitrogen atoms is protected with a suitable nitrogen protecting group PG, such as a tert-butoxycarbonyl group. Subsequently, the remaining leaving group is substituted with a second substituent, such as an alkoxy or alkyl group. The order of these substitution reactions is dependent on the particular target. In Step D, the protecting groups are removed under standard conditions. The R¹ group is introduced in Step E, for example by treatment of intermediate 6 with an acid anhydride in the presence of a base such as diisopropylethylamine in a solvent such as dichloromethane. In some instances a second step is required to remove superfluous acryloyl moieties.

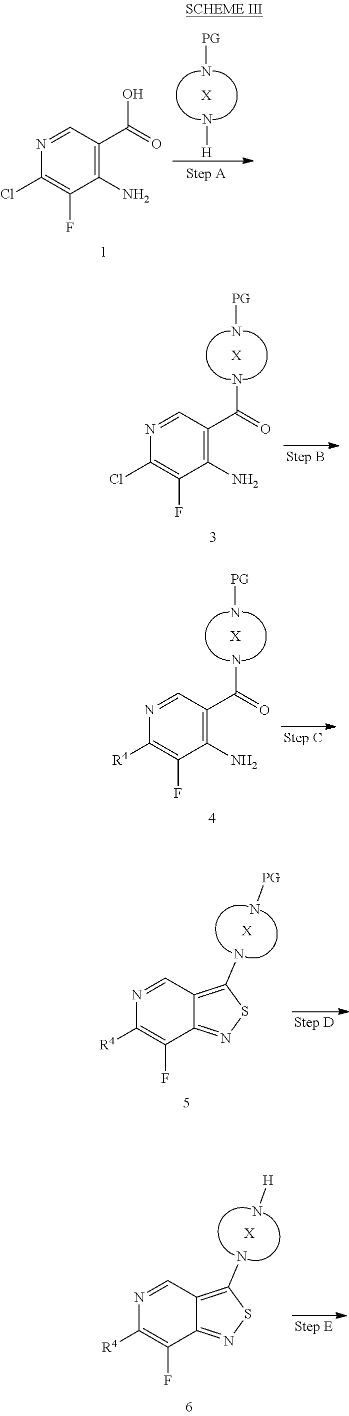

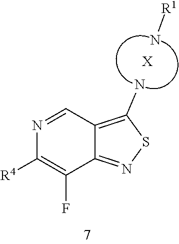

Compounds of Formula (I) where R² and R³ are absent and Y is S, can be prepared according to general Scheme III. A suitably substituted compound 1 is reacted in Step A with a heterocycle, wherein one of the nitrogen atoms is protected with a suitable nitrogen protecting group PG, such as a tert-butoxycarbonyl group. In Step B, coupling of an R⁴ group is accomplished by using a suitably functionalized R⁴, for example a boronic acid or boronate ester, in the presence of a palladium catalyst and a base such as potassium phosphate in a solvent such as dioxane. In Step C, conversion of the carbonyl moiety to the thiocarbonyl group and ring annulation is achieved. In Step D, the protecting groups are removed under standard conditions. The R¹ group is introduced in Step E, for example by treatment of intermediate 6 with an acid anhydride in the presence of a base such as diisopropylethylamine in a solvent such as dichloromethane.

The following intermediates may be used to synthesize compounds in Schemes I-III:

### Intermediate A-1

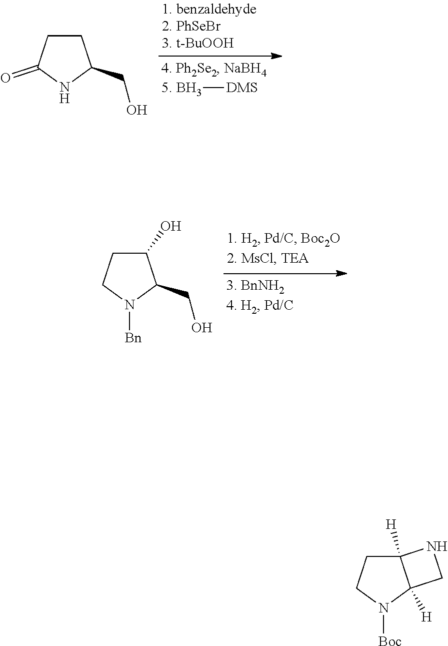

To a mixture of (S)-5-(hydroxymethyl)pyrrolidin-2-one (7.26 g, 63.1 mmol, 1.0 equiv) in toluene (150 mL) was added benzaldehyde (7.01 mL, 69.4 mmol, 1.1 equiv), TsOH·H₂O (163 mg, 946 μmol, 0.015 equiv) under N₂. The mixture was stirred at 125° C. for 48 h and was then diluted with water (100 mL) and extracted with ethyl acetate (300 mL). The combined organic layer was washed with saturated aq NaHCO₃ (300 mL), dried over anh sodium sulfate, filtered and concentrated under vacuum to provide the crude residue. The residue was purified by column chromatography (SiO₂, petroleum ether/ethyl acetate, 20:1 to 1:1) to afford (7aS)-3-phenyltetrahydro-3H,5H-pyrrolo[1,2-c]oxazol-5-one (5.70 g, 33% yield) as a brown oil. LCMS [ESI, M+1]: 204.

To a solution of (7aS)-3-phenyltetrahydro-3H,5H-pyrrolo[1,2-c]oxazol-5-one (5.00 g, 24.6 mmol, 1.0 equiv) in THE (50.0 mL) at −65° C. was added LiHMDS (1 M, 49.2 mL, 2.0 equiv). The mixture was stirred at this temperature for 30 min prior to the dropwise addition of PhSeBr (6.39 g, 27.1 mmol, 1.1 equiv) in THE (15.0 mL). The mixture was stirred at −65° C. for 1 h and was diluted with satd aq NH₄Cl (100 mL) and extracted with ethyl acetate (200 mL). The combined organic layer was dried over anh sodium sulfate, filtered and concentrated under vacuum. The resultant residue was dissolved in DCM (120 mL) at 0° C. and to this solution was added H₂O₂ (16.7 g, 148 mmol, 14.2 mL, 30% in water, 6.0 equiv). The mixture was stirred at 25° C. for 3 h and was then diluted with DCM (50 mL) and washed with HCl (150 mL, 1 M), satd aq NaHCO₃ (150 mL), and satd aq Na₂S₂O₃ (150 mL). The organic layer was dried over anh sodium sulfate, filtered and concentrated under vacuum. The resultant residue was purified by column chromatography (SiO₂, petroleum ether/ethyl acetate, 20:1 to 1:1) to afford (7aS)-3-phenyl-1,7a-dihydro-3H,5H-pyrrolo[1,2-c]oxazol-5-one (3.30 g, 63% yield) as a yellow solid. ¹H NMR (400 MHz, CDCl₃): δ 7.45 (d, J=7.2 Hz, 2H), 7.35-7.24 (m, 3H), 7.19 (dd, J=1.6, 5.6 Hz, 1H), 6.17-6.04 (m, 2H), 4.61-4.49 (m, 1H), 4.19 (t, J=7.6 Hz, 1H), 3.35 (t, J=8.0 Hz, 1H). LCMS [ESI, M+1]: 202.

To a solution of (7aS)-3-phenyl-1,7a-dihydro-3H,5H-pyrrolo[1,2-c]oxazol-5-one (2.80 g, 13.9 mmol, 1.0 equiv) in DMF (50.0 mL) was added K₂CO₃ (1.92 g, 13.9 mmol, 1.0 equiv) and 70% t-butyl hydroperoxide (5.52 g, 61.2 mmol, 5.87 mL, 4.4 equiv) in portions under N₂. The mixture was stirred at 25° C. for 30 min prior to the addition of Bu₄NF·3H₂O (13.2 g, 41.7 mmol, 3.0 equiv). The mixture was stirred at room temperature for 1 h prior to being diluted with satd aq NH₄Cl (50 mL) and extracted with MTBE (2×100 mL). The organic layer was washed with water (3×100 mL), dried over anh Na₂SO₄, filtered and concentrated under vacuum. The resultant residue was purified by column chromatography (SiO₂, petroleum ether/ethyl acetate, 20:1 to 1:1) to afford (1aR,1bR,6aR)-4-phenyltetrahydro-4H,6H-oxireno[2′,3′:3,4]pyrrolo[1,2-c]oxazol-6-one (1.20 g, 38% yield, 94.8% purity) as a brown solid. ¹H NMR (400 MHz, CDCl₃): δ 7.42-7.31 (m, 5H), 6.34 (s, 1H), 4.29-4.17 (m, 2H), 4.06 (d, J=2.4 Hz, 1H), 3.81 (d, J=2.0 Hz, 1H), 3.56 (dd, J=7.6, 8.4 Hz, 1H). LCMS [ESI, M+1]: 218.

To a solution of PhSeSePh (2.09 g, 6.70 mmol, 1.5 equiv) in EtOH (20.0 mL) at 0° C. was added NaBH₄ (506 mg, 13.4 mmol, 3.0 equiv) and the mixture was stirred for 15 min prior to the addition of HOAc (1.21 g, 20.1 mmol, 1.15 mL, 4.5 equiv). The resultant solution was added to (1aR,1bR,6aR)-4-phenyltetrahydro-4H,6H-oxireno[2′,3′:3,4]pyrrolo[1,2-c]oxazol-6-one (1.20 g, 4.46 mmol, 1.0 equiv) in EtOH (12.0 mL) and stirred at 25° C. for 30 min. The reaction mixture was diluted with EtOAc (150 mL) and oxygen gas was bubbled through for 5 min. The residue was concentrated under vacuum and the resultant residue was purified by column chromatography (SiO₂, petroleum ether/ethyl acetate, 3:1 to 0:1) to afford (7S,7aR)-7-hydroxy-3-phenyltetrahydro-3H,5H-pyrrolo[1,2-c]oxazol-5-one (1.06 g, crude) as a brown solid. LCMS [ESI, M+1]: 220.

To a solution of (7S,7aR)-7-hydroxy-3-phenyltetrahydro-3H,5H-pyrrolo[1,2-c]oxazol-5-one (1.39 g, 6.34 mmol, 1.0 equiv) in THE (25.0 mL) was added BH₃-Me₂S (10 M, 6.34 mL, 10 equiv). The reaction mixture was stirred at 70° C. for 2 h and then cooled to room temperature. The reaction mixture was quenched with HCl (4 M, 12 mL) and stirred at 70° C. for 1 h. The mixture was diluted with saturated aq Na₂CO₃ and was extracted with ethyl acetate (3×100 mL). The combined organic layer was dried over anh Na₂SO₄, filtered and concentrated under vacuum to afford (2R,3S)-1-benzyl-2-(hydroxymethyl)pyrrolidin-3-ol (1.55 g, 70% yield) as a colorless oil. LCMS [ESI, M+1]: 208.

To a solution of (2R,3S)-1-benzyl-2-(hydroxymethyl)pyrrolidin-3-ol (500 mg, 1.43 mmol, 1.0 equiv) and tert-butoxycarbonyl tert-butyl carbonate (933 mg, 4.28 mmol, 982 μL, 3.0 equiv) in MeOH (50.0 mL) was added Pd/C (500 mg, 334 μmol, 10 wt %). The mixture was stirred at 40° C. for 16 h under H₂ (50 psi). The system was flushed with nitrogen and the mixture was filtered and the filtrate was concentrated under reduced pressure. The resultant residue was purified by column chromatography (SiO₂, petroleum ether/ethyl acetate, 5:1 to dichloromethane/methanol, 10:1) to afford tert-butyl (2R,3S)-3-hydroxy-2-(hydroxymethyl)pyrrolidine-1-carboxylate (380 mg, crude) as a yellow oil. ¹H NMR (400 MHz, CDCl₃): δ 4.56 (br s, 1H), 4.20 (br s, 1H), 3.85-3.62 (m, 2H), 3.44-3.03 (m, 2H), 2.12-1.94 (m, 1H), 1.92-1.79 (m, 1H), 1.46 (s, 9H).

To a solution of tert-butyl (2R,3S)-3-hydroxy-2-(hydroxymethyl)pyrrolidine-1-carboxylate (380 mg, 1.75 mmol, 1.0 equiv) in DCM (10.0 mL) at 0° C. was added TEA (708 mg, 7.00 mmol, 974 μL, 4.0 equiv) and MsCl (501 mg, 4.37 mmol, 338 μL, 2.5 equiv). The mixture was stirred at 0° C. for 4 h and was then concentrated under vacuum. The resultant residue was diluted with ethyl acetate (20 mL) and washed with satd aq NaHCO₃ (10 mL). The organic layer was dried over anh Na₂SO₄, filtered and concentrated under vacuum. The residue was purified by column chromatography (SiO₂, petroleum ether/ethyl acetate, 20:1 to 1:1) to afford tert-butyl (2R,3S)-3-((methylsulfonyl)oxy)-2-(((methylsulfonyl)oxy)methyl)pyrrolidine-1-carboxylate (680 mg, crude) as a colorless oil. ¹H NMR (400 MHz, CD₃OD): δ 5.24 (br s, 1H), 4.46-4.27 (m, 2H), 4.19 (br s, 1H), 3.59-3.45 (m, 2H), 3.19-3.09 (m, 6H), 2.44-2.30 (m, 1H), 2.29-2.18 (m, 1H), 1.49 (br s, 9H). LCMS [ESI, M-99]: 274.

To a solution of tert-butyl (2R,3S)-3-((methylsulfonyl)oxy)-2-(((methylsulfonyl)oxy)methyl)pyrrolidine-1-carboxylate (680 mg, 1.82 mmol, 1.0 equiv) in toluene (10.0 mL) was added benzyl amine (611 mg, 5.70 mmol, 621 μL, 3.13 equiv). The mixture was stirred at 110° C. for 16 h and then cooled to room temperature. The mixture was concentrated under vacuum and the residue was diluted with DCM (20 mL). The organic layer was washed with 1 N NaOH, was dried over anh Na₂SO₄, filtered and concentrated under vacuum. The resultant residue was purified by column chromatography (SiO₂, petroleum ether/ethyl acetate, 20:1 to 3:1) to afford tert-butyl (1R,5R)-6-benzyl-2,6-diazabicyclo[3.2.0]heptane-2-carboxylate (320 mg, 58% yield) as a colorless oil. ¹H NMR (400 MHz, CDCl₃): δ 7.25-7.14 (m, 5H), 4.27-4.07 (m, 1H), 3.81 (t, J=4.8 Hz, 1H), 3.73-3.51 (m, 4H), 3.29-3.11 (m, 1H), 3.10-2.95 (m, 1H), 1.60-1.44 (m, 2H), 1.37 (br d, J=16.8 Hz, 9H). LCMS [ESI, M+1]: 289.

To a solution of tert-butyl (1R,5R)-6-benzyl-2,6-diazabicyclo[3.2.0]heptane-2-carboxylate (320 mg, 1.11 mmol, 1.0 equiv) in EtOH (10.0 mL) was added Pd/C (150 mg, 10 wt. %). The mixture was stirred at 60° C. for 36 h under a hydrogen atmosphere (50 psi). The system was flushed with nitrogen and the mixture was filtered. The filtrate was concentrated under vacuum to provide the crude residue. The residue was purified by column chromatography (Al₂O₃, petroleum ether/ethyl acetate, 5:1 to 1:1 to ethyl acetate/methanol, 100:1 to 10:1) to afford tert-butyl (1R,5R)-2,6-diazabicyclo[3.2.0]heptane-2-carboxylate (80.0 mg, 36% yield) as a colorless oil. ¹H NMR (400 MHz, CDCl₃): δ 4.63 (br t, J=5.2 Hz, 1H), 4.57-4.34 (m, 1H), 3.95-3.59 (m, 3H), 3.32-3.05 (m, 2H), 1.95-1.71 (m, 2H), 1.48-1.43 (m, 9H).

### Intermediates A-2 & A-3

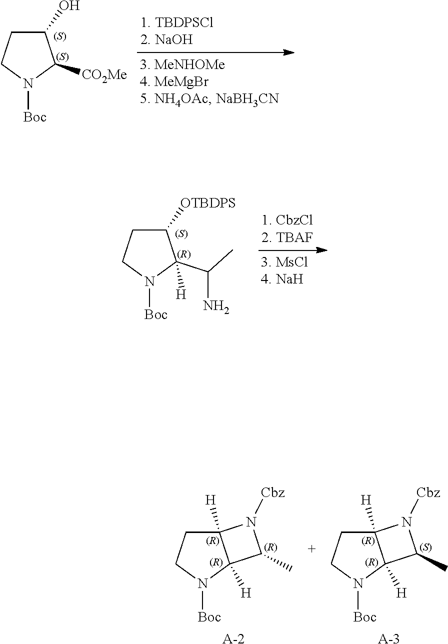

To a mixture of compound 1-(tert-butyl) 2-methyl (2S,3S)-3-hydroxypyrrolidine-1,2-dicarboxylate (100 g, 407.7 mmol, 1.00 equiv) in DMF (1.3 L) at 0° C. was added DMAP (4.98 g, 40.7 mmol, 0.100 equiv), TBDPSCl (126 mL, 489.3 mmol, 1.2 equiv) and imidazole (138.8 g, 2.04 mol, 5.0 equiv). The mixture was stirred at 20° C. for 2 h prior to being poured into water (2500 mL) and extracted with EtOAc (1000 mL×3). The combined organic layer was washed with brine (1500 mL×3), dried with anhydrous Na₂SO₄, filtered and concentrated in vacuum. The crude product was purified by silica gel column chromatography (petroleum ether/ethyl acetate, 50:1 to 10:1) to give 1-(tert-butyl) 2-methyl (2S,3S)-3-((tert-butyldiphenylsilyl)oxy)pyrrolidine-1,2-dicarboxylate (90 g, 186.1 mmol, 45.6% yield) as a yellow oil. LCMS [ESI, M+1]: 384.2; ¹H NMR (400 MHz, CDCl₃): δ 7.66-7.64 (m, 4H), 7.41-7.39 (m, 6H), 4.39-4.38 (m, 1H), 4.33-4.18 (m, 1H), 3.64-3.58 (m, 5H), 1.84-1.80 (m, 2H), 1.65-1.41 (m, 9H), 1.03 (s, 9H).

The mixture of compound 1-(tert-butyl) 2-methyl (2S,3S)-3-((tert-butyldiphenylsilyl)oxy)pyrrolidine-1,2-dicarboxylate (90.0 g, 186.1 mmol, 1.00 equiv) in EtOH (200 mL) was added the solution of NaOH (14.9 g, 372.2 mmol, 2.00 equiv) in water (20 mL). The mixture was stirred at room temperature for 1 h prior to being poured into water (500 mL) and adjusted to pH 6 with 0.1 M HCl. The mixture partially concentrated to remove the EtOH and then was extracted with EtOAc (150 mL×3). The organic layer was dried over anh Na₂SO₄, filtered and concentrated to dryness to afford (2S,3S)-1-(tert-butoxycarbonyl)-3-((tert-butyldiphenylsilyl)oxy)pyrrolidine-2-carboxylic acid (88 g) as a colorless oil. ¹H NMR (400 MHz, CDCl₃): δ 7.73-7.43 (m, 4H), 7.40-7.37 (m, 6H), 4.75-4.47 (m, 1H), 4.25-4.12 (m, 1H), 3.68-3.43 (m, 2H), 1.84-1.82 (m, 2H), 1.52-1.40 (m, 8H), 1.23 (s, 9H).

To a mixture of (2S,3S)-1-(tert-butoxycarbonyl)-3-((tert-butyldiphenylsilyl)oxy)pyrrolidine-2-carboxylic acid (82.0 g, 174.6 mmol, 1.00 equiv) and N-methoxymethanamine (16.5 g, 168.6 mmol, 1.20 equiv, HCl salt) in ACN (600 mL) was added Et₃N (59 mL, 421.6 mmol, 3.00 equiv) and T₃P (125 mL, 211 mmol, 50.0% in EtOAc, 1.50 equiv). The reaction mixture was stirred at 20° C. for 10 h prior to being concentrated, poured into water (3 L) and extracted with EtOAc (1000 mL×3). The combined organic layer was washed with water (2000 mL×3), dried over anh Na₂SO₄, filtered and concentrated to dryness. The residue was purified by silica gel chromatography (petroleum ether/ethyl acetate, 10:1 to 1:1) to provide compound tert-butyl (2S,3S)-3-((tert-butyldiphenylsilyl)oxy)-2-(methoxy(methyl)carbamoyl)pyrrolidine-1-carboxylate (63.0 g, 122.9 mmol, 87.4% yield) as a yellow oil. LCMS [ESI, M-100]: 413.2; ¹H NMR (400 MHz, CDCl₃): δ 7.69-7.66 (m, 4H), 7.44-7.39 (m, 6H), 4.85-4.82 (m, 1H), 4.36-4.33 (m, 1H), 3.72-3.69 (m, 2H), 3.63-3.53 (m, 3H), 3.38-3.06 (m, 3H), 1.89-1.83 (m, 2H), 1.74-1.50 (d, 9H) 1.03 (s, 9H).

To a solution of compound tert-butyl (2S,3S)-3-((tert-butyldiphenylsilyl)oxy)-2-(methoxy(methyl)carbamoyl)pyrrolidine-1-carboxylate (60 g, 117.0 mmol) in THE (200 mL) at −70° C. was added MeMgBr (3.00 M, 78.02 mL, 2.00 equiv) under an atmosphere of nitrogen. The mixture allowed to warm to room temperature and stirred for 10 h. The reaction mixture was diluted with NH₄Cl (1.50 L) and extracted with EtOAc (500 mL×3). The combined organic layer was dried over anh Na₂SO₄, filtered and concentrated to dryness. The residue was purified by silica gel chromatography (petroleum ether/ethyl acetate, 20:1 to 3:1) to provide tert-butyl (2S,3S)-2-acetyl-3-((tert-butyldiphenylsilyl)oxy)pyrrolidine-1-carboxylate (54 g, 115.5 mmol, 98.7% yield) as a yellow oil.

To a mixture of tert-butyl (2S,3S)-2-acetyl-3-((tert-butyldiphenylsilyl)oxy)pyrrolidine-1-carboxylate (46 g, 98.36 mmol, 1.00 equiv) in MeOH (500 mL) was added NH₄OAc (303 g, 3.93 mol, 40 equiv) and NaBH₃CN (7.42 g, 118.0 mmol, 1.2 equiv). The mixture was stirred at 85° C. for 1 h prior to being concentrated. To the residue was added water (1.00 L) and the mixture was extracted with EtOAc (500 mL×3). The combined organic layer was dried over anh sodium sulfate, filtered and concentrated to dryness to provide tert-butyl (2R,3S)-2-(1-aminoethyl)-3-((tert-butyldiphenylsilyl)oxy)pyrrolidine-1-carboxylate (51 g) as a light-yellow oil. LCMS [ESI, M+1]: 469.2.

To a mixture of tert-butyl (2R,3S)-2-(1-aminoethyl)-3-((tert-butyldiphenylsilyl)oxy)pyrrolidine-1-carboxylate (57 g, 121.61 mmol, 1.00 equiv) and Et₃N (51 mL, 364.8 mmol, 3.00 equiv) in DCM (500 mL) was added CbzCl (18 mL, 123 mmol, 1.0 equiv). The mixture was stirred at 20° C. for 1 h prior to being diluted with water (1000 mL) and extracted with EtOAc (500 mL×3). The organic layer was dried over anhydrous Na₂SO₄, filtered and concentrated. The residue was purified by silica gel chromatography (petroleum ether/ethyl acetate, 10:1 to 3:1) to afford tert-butyl (2R,3S)-2-(1-(((benzyloxy)carbonyl)amino)ethyl)-3-((tert-butyldiphenylsilyl)oxy)pyrrolidine-1-carboxylate (25 g, 41.47 mmol, 34% yield) as a yellow oil. LCMS [ESI, M+1]: 603.4.

To a mixture of tert-butyl (2R,3S)-2-(1-(((benzyloxy)carbonyl)amino)ethyl)-3-((tert-butyldiphenylsilyl)oxy)pyrrolidine-1-carboxylate (25.00 g, 41.5 mmol, 1.00 equiv) in DMF (200 mL) was added TBAF (1.00 M in THF, 50 mL, 1.2 equiv). The mixture was stirred at 20° C. for 2 h prior to being diluted with water (500 mL) and extracted with EtOAc (200 mL×3). The combined organic layer was washed with brine (300 mL), dried over anhydrous Na₂SO₄, filtered and concentrated. The residue was purified by silica gel chromatography (petroleum ether/ethyl acetate, 10:1 to 2:1) to afford tert-butyl (2R,3S)-2-(1-(((benzyloxy)carbonyl)amino)ethyl)-3-hydroxypyrrolidine-1-carboxylate (7.00 g, 19.2 mmol, 46.3% yield) as a yellow oil. LCMS [ESI, M+1]: 364.2.

To a mixture of tert-butyl (2R,3S)-2-(1-(((benzyloxy)carbonyl)amino)ethyl)-3-hydroxypyrrolidine-1-carboxylate (7.00 g, 19.2 mmol, 1.00 equiv) and TEA (5.4 mL, 38 mmol, 2.0 equiv) in DCM (100 mL) was added MsCl (3.0 mL, 38 mmol, 2.0 equiv). The mixture was stirred at room temperature for 60 h prior to being diluted with water (150 mL). The mixture was extracted with DCM (50 mL×3). The combined organic layer was washed with water (50 mL×3), dried over anh sod sulfate, filtered and concentrated. The residue was purified by silica gel chromatography (petroleum ether/ethyl acetate, 10:1 to 1:1) and then by reverse phase chromatography [Phenomenex Luna C18 250×50 mm×10 μm; A: water (0.225% FA), B: ACN; B %: 30%-60%, 15 min] to give tert-butyl (2R,3S)-2-(1-(((benzyloxy)carbonyl)amino)ethyl)-3-((methylsulfonyl)oxy)pyrrolidine-1-carboxylate (8.2 g) as a yellow oil. LCMS [ESI, M+1]: 343.0.

To the mixture of NaH (1.30 g, 32.5 mmol, 60.0% purity, 2.0 equiv) in DMF (70.0 mL) at room temperature was added dropwise a solution of tert-butyl (2R,3S)-2-(1-(((benzyloxy)carbonyl)amino)ethyl)-3-((methylsulfonyl)oxy)pyrrolidine-1-carboxylate (7.20 g, 16.3 mmol, 1.00 equiv) in DMF (20 mL). The mixture was stirred at room temperature for 1 h prior to being diluted with satd aq NaHCO₃ (200 mL) and extracted with EtOAc (200 mL×3). The organic layer was dried over anhydrous Na₂SO₄, filtered and concentrated. The residue was purified by prep-HPLC [Phenomenex Luna C18 (250×70 mm, 10 μm); A: water (0.1% TFA), B: ACN; B %: 40%-70%, 20 min] and then 4 g of the mixture by chiral SFC [DAICEL CHIRALPAK IG (250 mm×30 mm, 10 μm); A: MeOH (0.1% NH₄OH), B: CO₂; B %: 40%, 4.5 min] to provide 6-benzyl 2-(tert-butyl) (1R,5R,7R)-7-methyl-2,6-diazabicyclo[3.2.0]heptane-2,6-dicarboxylate (A-2, 1^{st} eluting) (1.5 g) as a yellow oil. ¹H NMR (400 MHz, CDCl₃): δ 7.37-7.27 (m, 5H), 5.10 (m, 2H), 4.80-4.77 (m, 1H), 4.48-4.43 (m, 2H), 4.15-3.96 (m, 1H), 3.46-3.41 (m, 1H), 3.19-3.16 (m, 1H), 2.14-2.05 (m, 1H), 1.76-1.72 (m, 1H) 1.47-1.45 (m, 9H), 1.26-1.19 (m, 3H). The second eluting peak, 6-benzyl 2-(tert-butyl) (1R,5R,7S)-7-methyl-2,6-diazabicyclo[3.2.0]heptane-2,6-dicarboxylate (A-3) was obtained as a yellow oil. ¹H NMR (400 MHz, CDCl₃): δ 7.38-7.27 (m, 5H), 5.17-5.13 (m, 2H), 4.87-4.86 (m, 1H), 4.03-3.79 (m, 3H), 3.47 (s, 1H), 2.41-2.36 (m, 1H), 1.84-1.81 (m, 1H), 1.78-1.26 (m, 12H).

### Intermediate A-4

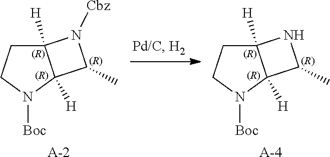

A mixture of 6-benzyl 2-(tert-butyl) (1R,5R,7R)-7-methyl-2,6-diazabicyclo[3.2.0]heptane-2,6-dicarboxylate (A-2) (600 mg, 1.73 mmol, 1.00 equiv) and Pd/C (200 mg, 10.0 wt. %) in MeOH (10.0 mL) was stirred under H₂ (15.0 psi) at 15° C. for 1 h. The vessel was flushed with nitrogen and reaction mixture was filtered and the filtrate was concentrated to afford tert-butyl (1R,5R,7R)-7-methyl-2,6-diazabicyclo[3.2.0]heptane-2-carboxylate (A-4) (278.9 mg, 1.24 mmol, 71.6% yield, 94% purity) as a white solid. ¹H NMR (400 MHz, CDCl₃): δ 4.37 (s, 1H), 3.86-3.78 (m, 1H), 3.59-3.49 (m, 2H), 3.19-3.16 (m, 1H), 1.68-1.64 (m, 2H), 1.39-1.36 (m, 9H), 1.23-1.21 (m, 3H).

### Intermediate A-5

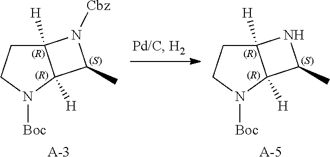

Procedure as with A-4: tert-butyl (1R,5R,7S)-7-methyl-2,6-diazabicyclo[3.2.0]heptane-2-carboxylate (A-5) as a white solid; LCMS [ESI, M+1]: 213.3; ¹H NMR (400 MHz, CDCl₃): δ 4.30-4.16 (m, 2H), 3.87-3.68 (m, 1H), 3.40-3.38 (m, 1H), 3.36-3.22 (m, 1H), 1.55-1.52 (m, 2H), 1.47-1.36 (d, 9H), 0.84-0.79 (m, 3H).

### Intermediate B-1

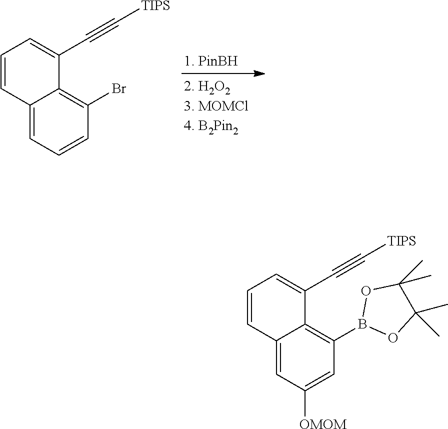

To a solution of ((8-bromonaphthalen-1-yl)ethynyl)triisopropylsilane (1.50 g, 3.87 mmol, 1.00 equiv) in THE (15.0 mL) was added dtbbpy (125 mg, 465 μmol, 0.12 equiv), (1,5-cyclooctadiene)(methoxy)iridium(I) dimer (257 mg, 387 μmol, 0.10 equiv) and 4,4,5,5-tetramethyl-1,3,2-dioxaborolane (1.24 g, 9.68 mmol, 1.40 mL, 2.50 equiv) under an atmosphere of argon. The mixture was stirred at 60° C. for 10 h and was concentrated under reduced pressure to afford a mixture of two borylation isomers (15.0 g, crude).

To a solution of the crude mixture of borylation isomers (15.0 g, 29.2 mmol, 1.00 equiv) in H₂O (20.0 mL) and THE (60.0 mL) was added H₂O₂ (29.8 g, 263 mmol, 25.3 mL, 9.00 equiv) and acetic acid (121 g, 2.02 mol, 115 mL, 69.0 equiv), the mixture was stirred at 10° C. for 1 h prior to being diluted with satd aq NaHSO₃ (300 mL). The mixture was extracted with ethyl acetate (3×200 mL). The combined organic layer was washed with brine (200 mL), dried over anhydrous Na₂SO₄, filtered and concentrated under reduced pressure. The mixture was purified by column chromatography (SiO₂, petroleum ether/ethyl acetate, 1:0 to 20:1), then by prep-HPLC [column: Phenomenex Luna C18 (250*70 mm, 10 μm); mobile phase: water (0.225% FA); ACN: 70%-99%, 40 min], and then by SFC separation [column: DAICEL CHIRALPAK AD (250 mm*30 mm, 10 μm); mobile phase: (0.1% NH₄OH in IPA)] to afford 4-bromo-5-((triisopropylsilyl)ethynyl)naphthalen-2-ol (3.00 g, 7.44 mmol, 13% yield) as a light yellow solid. ¹H NMR (400 MHz, CDCl₃): δ 7.73-7.72 (m, 1H), 7.64-7.51 (m, 1H), 7.49 (d, J=2.8 Hz, 1H), 7.35-7.32 (m, 1H), 7.12 (d, J=2.8 Hz, 1H), 1.20-1.16 (m, 21H).

To a solution of 4-bromo-5-((triisopropylsilyl)ethynyl)naphthalen-2-ol (2.90 g, 7.19 mmol, 1.00 equiv) and DIEA (2.79 g, 21.6 mmol, 3.76 mL, 3.00 equiv) in DCM (3.00 mL) at 0° C. was added dropwise MOMCl (1.10 g, 13.7 mmol, 1.04 mL, 1.90 equiv). The mixture was stirred at 0° C. for 0.5 h prior to being diluted with H₂O (40 mL). The mixture was extracted with DCM (90 mL). The organic layer was washed with brine (20 mL), dried over anh Na₂SO₄, filtered and concentrated under reduced pressure to give a residue. The residue was purified by column chromatography (SiO₂, petroleum ether/ethyl acetate, 1:0 to 50:1) to afford ((8-bromo-6-(methoxymethoxy)naphthalen-1-yl)ethynyl)triisopropylsilane (2.00 g, 4.47 mmol, 62% yield) as a light yellow solid. ¹H NMR (400 MHz, CDCl₃): δ 7.75 (dd, J=1.2, 7.2 Hz, 1H), 7.72-7.65 (m, 1H), 7.60 (d, J=2.4 Hz, 1H), 7.39-7.30 (m, 2H), 5.27 (s, 2H), 3.52 (s, 3H), 1.21-1.15 (m, 21H).

To a mixture of ((8-bromo-6-(methoxymethoxy)naphthalen-1-yl)ethynyl)triisopropylsilane (400 mg, 893 umol, 1.00 equiv), 4,4,5,5-tetramethyl-2-(4,4,5,5-tetramethyl-1,3,2-dioxaborolan-2-yl)-1,3,2-dioxaborolane (454 mg, 1.79 mmol, 2.00 equiv) and KOAc (263 mg, 2.68 mmol, 3.00 equiv) in toluene (8.00 mL) was added Pd(dppf)Cl₂ (196 mg, 268 umol, 0.30 equiv) under an atmosphere of nitrogen. The mixture was stirred at 80° C. for 12 h and then cooled to room temperature. The mixture was filtered and the filtrate was concentrated in vacuo. The residue was purified by prep-TLC (petroleum ether/ethyl acetate, 5:1, Rf=0.5) to afford triisopropyl((8-(4,4,5,5-tetramethyl-1,3,2-dioxaborolan-2-yl)naphthalen-1-yl)ethynyl)silane (390 mg, 787 μmol, 88% yield) as a light yellow solid. ¹H NMR (400 MHz, CDCl₃): δ 7.69 (dd, J=3.2, 7.6 Hz, 2H), 7.47 (d, J=2.8 Hz, 1H), 7.40-7.31 (m, 2H), 5.29 (s, 2H), 3.51 (s, 3H), 1.44 (s, 12H), 1.20-1.12 (m, 21H).

### Intermediate B-2

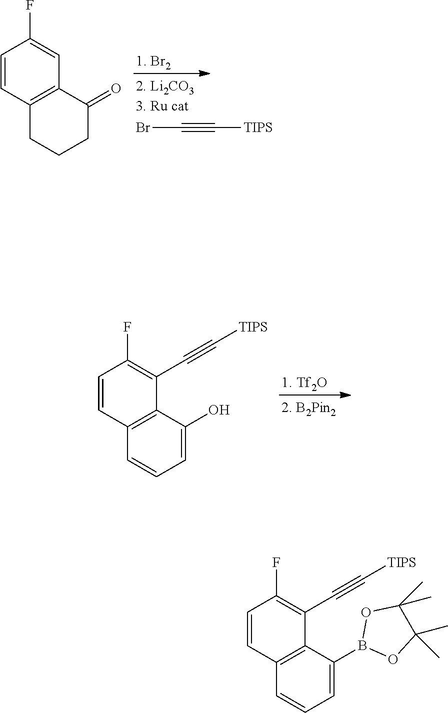

To a solution of 7-fluoro-3,4-dihydronaphthalen-1(2H)-one (75.0 g, 457 mmol, 1.00 equiv) in acetic acid (1.50 L) and hydrogen bromide (33% in acetic acid, 7.50 mL) at 0° C. was added bromine (25.9 mL, 503 mmol, 1.1 equiv) in acetic acid (50 mL). The mixture was stirred at 25° C. for 3 hours. The mixture was diluted with DCM (1.5 L) and washed with water (3×500 mL). The organic layer was dried over anhydrous sodium sulfate, filtered and concentrated to afford a brown oil. The oil was dissolved in DMF (750 mL) and to this mixture was added lithium bromide (67.4 g, 777 mmol, 1.70 equiv) and lithium carbonate (57.4 g, 777 mmol, 1.70 equiv). The reaction mixture was stirred at 160° C. for 3.5 h and then cooled to room temperature. The mixture was extracted with ethyl acetate (1.00 L). The organic layer was washed with brine (2×500 mL), dried over anhydrous sodium sulfate, filtered and concentrated in vacuum to give a residue. The residue was purified by silica gel chromatography (petroleum ether/ethyl acetate, 1:0 to 5:1) to afford 7-fluoronaphthalen-1-ol (61.0 g, 82% yield) as a brown solid. ¹H NMR (400 MHz, CDCl₃): δ 7.84-7.77 (m, 2H), 7.44 (d, J=8.0 Hz, 1H), 7.31-7.24 (m, 2H), 6.84 (d, J=7.6 Hz, 1H), 5.39 (s, 1H).

To a solution of 7-fluoro-3,4-dihydronaphthalen-1(2H)-one (72.0 g, 275 mmol, 1.20 equiv) and 7-fluoronaphthalen-1-ol (37.2 g, 230 mmol, 1.0 equiv) in DCE (500 mL) was added (p-cymene)ruthenium(II) chloride dimer (21.1 g, 34.4 mmol, 0.15 equiv), K₂CO₃ (31.7 g, 230 mmol, 1.0 equiv) and NaOAc (3.77 g, 45.9 mmol, 0.20 equiv). The mixture was stirred at 40° C. for 12 hours. The reaction mixture was filtered and concentrated under reduced pressure to give a residue. The residue was purified by column chromatography (SiO₂, petroleum ether/ethyl acetate, 1:0 to 50:1) to afford 7-fluoro-8-((triisopropylsilyl)ethynyl)naphthalen-1-ol (73.0 g, 93% yield) as a yellow oil. ¹H NMR (400 MHz, CDCl₃): δ 9.10 (s, 1H), 7.79 (dd, J=5.6, 8.8 Hz, 1H), 7.41-7.33 (m, 2H), 7.23 (t, J=8.8 Hz, 1H), 7.08-7.00 (m, 1H), 1.24-1.14 (m, 21H). LCMS [ESI, M+1, 2M+1]: 343.1.

To a solution of 7-fluoro-8-((triisopropylsilyl)ethynyl)naphthalen-1-ol (73.0 g, 213 mmol, 1.00 equiv) in DCM (600 mL) at −40° C. was added DIEA (55.1 g, 426 mmol, 74.2 mL, 2.00 equiv) and Tf₂O (90.2 g, 320 mmol, 52.7 mL, 1.50 equiv). The mixture was stirred at this temperature for 30 min prior to being filtered and concentrated under reduced pressure to give a residue. The residue was purified by column chromatography (SiO₂, petroleum ether/ethyl acetate, 1:0 to 50:1) to provide 7-fluoro-8-((triisopropylsilyl)ethynyl)naphthalen-1-yl trifluoromethanesulfonate (78.0 g, 77% yield) as a yellow solid. ¹H NMR (400 MHz, CDCl₃): δ 7.88-7.79 (m, 2H), 7.59-7.52 (m, 1H), 7.46 (t, J=8.0 Hz, 1H), 7.37 (t, J=8.8 Hz, 1H), 1.32-1.16 (m, 21H).

To a solution of 7-fluoro-8-((triisopropylsilyl)ethynyl)naphthalen-1-yl trifluoromethanesulfonate (20.0 g, 42.1 mmol, 1.00 equiv) and Pin₂B₂ (16.0 g, 63.2 mmol, 1.50 equiv) in dioxane (6.00 mL) was added KOAc (8.27 g, 84.3 mmol, 2.0 equiv) and Pd(dppf)Cl₂ (3.08 g, 4.21 mmol, 0.10 equiv). The mixture was stirred at 110° C. for 12 h under an atmosphere of nitrogen prior to being filtered and concentrated under reduced pressure. The resultant residue was purified by column chromatography (SiO₂, petroleum ether/ethyl acetate, 1:0 to 10:1) to afford ((2-fluoro-8-(4,4,5,5-tetramethyl-1,3,2-dioxaborolan-2-yl)naphthalen-1-yl)ethynyl)triisopropylsilane (9.0 g, 47% yield) as a yellow solid. LCMS [ESI, M+1]: 453.2; ¹H NMR (400 MHz, CDCl₃) δ 7.85-7.75 (m, 3H), 7.43 (dd, J=7.2, 8.0 Hz, 1H), 7.30-7.24 (m, 1H), 1.45 (s, 12H), 1.21-1.14 (m, 21H).

### Intermediate B-3

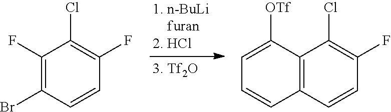 [SMILES: Fc1ccc(Br)c(F)c1Cl ; O=S(=O)(Oc1cccc2ccc(F)c(Cl)c12)C(F)(F)F]

To a mixture of 1-bromo-3-chloro-2,4-difluorobenzene (250 g, 1.10 mol, 1.00 equiv) and furan (150 g, 2.20 mol, 160 mL, 2.00 equiv) in toluene (2.50 L) at −15° C. was added n-BuLi (2.50 M, 528 mL, 1.2 equiv) dropwise over 0.5 hour. The mixture was allowed to warm to room temperature and stirring continued for 12 h. Subsequently, the mixture was quenched with water (2 L) and was filtered. The organic layer was collected and the aqueous layer was extracted with ethyl acetate (2 L×2). The combined organic layer was dried over anh Na₂SO₄ and filtered. The filtrate was concentrated under vacuum. The residue was purified by reversed phase flash [C18, 0.1% FA in water, 0-80% MeCN] to afford 5-chloro-6-fluoro-1,4-dihydro-1,4-epoxynaphthalene (81.0 g, 37% yield) as a yellow oil. ¹H NMR (400 MHz, CDCl₃): δ 7.11-7.06 (m, 2H), 7.06-7.01 (m, 1H), 6.73 (dd, J=7.6, 9.6 Hz, 1H), 5.88 (s, 1H), 5.74 (s, 1H).

A mixture of 5-chloro-6-fluoro-1,4-dihydro-1,4-epoxynaphthalene (162 g, 824 mmol, 1.00 equiv) in concentrated hydrochloric acid (1.00 L, 10.1 mol, 12.2 equiv) and ethyl alcohol (1.20 L) was heated at 80° C. with stirring for 6 h. Subsequently, the reaction mixture was concentrated under vacuum. The residue was adjusted to pH˜7 with saturated aq NaHCO₃ and then extracted with ethyl acetate (2 L×2). The combined organic layer was dried over anh Na₂SO₄ and filtered. The filtrate was concentrated under vacuum. The residue was triturated with petroleum ether (100 mL) and then filtered. The filter cake was dried under vacuum to afford 8-chloro-7-fluoronaphthalen-1-ol (124 g, 76% yield) as a white solid. ¹H NMR (400 MHz, CDCl₃): δ 7.92 (s, 1H), 7.75 (dd, J=5.2, 8.8 Hz, 1H), 7.44-7.36 (m, 2H), 7.33-7.26 (m, 1H), 7.12-7.06 (m, 1H).

A mixture of 8-chloro-7-fluoronaphthalen-1-ol (124 g, 631 mmol, 1.00 equiv), DIEA (489 g, 3.78 mol, 659 mL, 6.00 equiv), 4 Å MS (120 g) in dichloromethane (1.5 L) was stirred for 10 minutes at 20° C. To this suspension cooled to −40° C. was added dropwise trifluoromethylsulfonyl trifluoromethanesulfonate (231 g, 820 mmol, 135 mL, 1.30 equiv). After 20 min the reaction mixture was diluted with water (1 L) and the organic layer was collected. The aqueous layer was then extracted with ethyl acetate (1 L×2). The combined organic layer was dried over anh Na₂SO₄ and filtered. The filtrate was concentrated under vacuum. The residue was purified by silica gel chromatography (petroleum ether: ethyl acetate, 1:0 to 20:1) to afford 8-chloro-7-fluoronaphthalen-1-yl trifluoromethanesulfonate (196 g, 92% yield) as a yellow solid. ¹H NMR (400 MHz, CDCl₃): δ 7.86 (d, J=8.0 Hz, 1H), 7.83-7.76 (m, 1H), 7.57 (d, J=8.0 Hz, 1H), 7.53-7.44 (m, 1H), 7.43-7.35 (m, 1H).

### Intermediate B-4

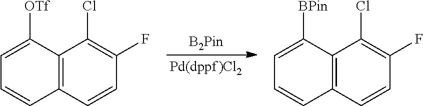

A mixture of 8-chloro-7-fluoronaphthalen-1-yl trifluoromethanesulfonate (27.0 g, 82.1 mmol, 1.00 equiv), (PinB)₂ (41.7 g, 164 mmol, 2.00 equiv), KOAc (40.3 g, 411 mmol, 5.00 equiv) and Pd(dppf)Cl₂ (6.01 g, 8.22 mmol, 0.10 equiv) in DMF (300 mL) was purged with nitrogen and then the mixture was stirred at 80° C. for 12 h. The mixture was cooled to room temperature, diluted with ethyl acetate (500 mL) and water (400 mL). The organic layer was separated and the aqueous layer was extracted with ethyl acetate (400 mL×2). The combined organic layer was washed with brine (800 mL), dried over anh Na₂SO₄ and filtered. The filtrate was concentrated under vacuum. The residue was purified by column chromatography (SiO₂, petroleum ether/ethyl acetate, 1:0 to 50:1) to afford 2-(8-chloro-7-fluoronaphthalen-1-yl)-4,4,5,5-tetramethyl-1,3,2-dioxaborolane (19 g, 74% yield) as a white solid. ¹H NMR (400 MHz, CDCl₃): δ 7.86 (dd, J=1.2, 8.4 Hz, 1H), 7.76 (dd, J=5.6, 9.2 Hz, 1H), 7.71 (d, J=6.8 Hz, 1H), 7.49 (dd, J=7.2, 8.0 Hz, 1H), 7.33 (t, J=8.8 Hz, 1H), 1.46 (s, 12H).

### Intermediate B-5

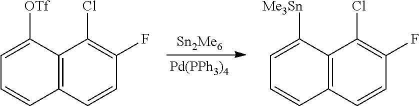

A mixture of (8-chloro-7-fluoro-1-naphthyl) trifluoromethanesulfonate (80.0 mg, 243 μmol, 1.0 equiv), trimethyl(trimethylstannyl)stannane (360 mg, 1.10 mmol, 228 μL, 4.5 equiv), Pd(PPh₃)₄ (28.1 mg, 24.3 μmol, 0.1 equiv), LiCl (61.9 mg, 1.46 mmol, 6.0 equiv) in toluene (1 mL) was purged with N₂ and then the mixture was stirred at 100° C. for 16 h. The reaction mixture was cooled to room temperature and filtered. The filtrate was diluted with water (5 mL) and extracted with ethyl acetate (2×10 mL). The combined organic layer was dried over anh Na₂SO₄ and concentrated under vacuum. The residue was purified by column chromatography (SiO₂, petroleum ether/ethyl acetate, 1:0 to 10:1) to afford (8-chloro-7-fluoro-1-naphthyl)-trimethyl-stannane (50.0 mg, 96.1 μmol, 39% yield) as a colorless oil. ¹H NMR (400 MHz, chloroform-d): δ 7.88 (d, J=6.8 Hz, 1H), 7.83 (dd, J=1.2, 8.0 Hz, 1H), 7.78 (dd, J=6.0, 9.2 Hz, 1H), 7.45 (dd, J=6.8, 8.0 Hz, 1H), 7.35 (t, J=8.8 Hz, 1H), 0.44 (s, 9H).

### Intermediate B-6

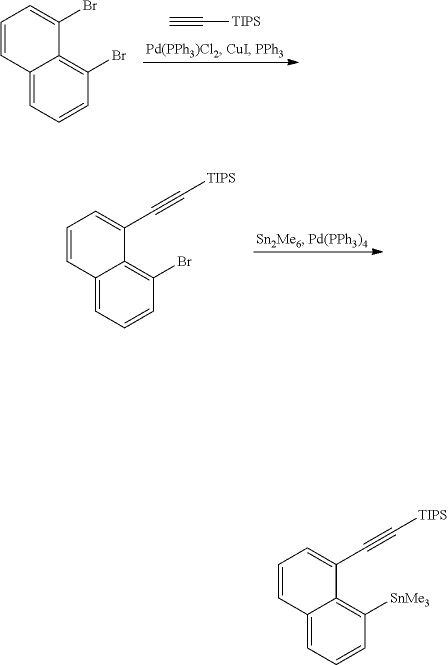

A mixture of 1,8-dibromonaphthalene (7 g, 24.5 mmol, 1.0 equiv), ethynyl(triisopropyl)silane (4.91 g, 26.9 mmol, 6.04 mL, 1.1 equiv), CuI (466 mg, 2.45 mmol, 0.1 equiv), PPh₃ (642 mg, 2.45 mmol, 0.1 equiv) and Pd(PPh₃)₂Cl₂ (859 mg, 1.22 mmol, 0.05 equiv) in TEA (100 mL) was stirred at 80° C. for 3 h under N₂. The mixture was cooled to room temperature and was diluted with water (50 mL) and extracted with ethyl acetate (2×50 mL). The combined organic layer was washed with brine (50 mL), dried over anh Na₂SO₄, filtered and concentrated under vacuum. The residue was purified by column chromatography (SiO₂, petroleum ether) to afford ((8-bromonaphthalen-1-yl)ethynyl)triisopropylsilane (7 g, 18.1 mmol, 74% yield) as a yellow solid. ¹H NMR (400 MHz, chloroform-d): 6=7.87 (dd, J=1.2, 7.2 Hz, 1H), 7.82-7.73 (m, 3H), 7.41-7.34 (m, 1H), 7.24 (t, J=7.6 Hz, 1H), 1.19-1.16 (m, 21H).

A mixture of ((8-bromonaphthalen-1-yl)ethynyl)triisopropylsilane (6.5 g, 16.8 mmol, 1.0 equiv), trimethyl(trimethylstannyl)stannane (27.5 g, 83.9 mmol, 17.4 mL, 5.0 equiv) and Pd(PPh₃)₄ (1.94 g, 1.68 mmol, 0.1 equiv) in toluene (100 mL) was stirred at 110° C. for 48 h under N₂. Subsequently, the mixture was diluted with water (100 mL) and the mixture was extracted with ethyl acetate (2×100 mL). The combined organic layer was washed with brine (100 mL), dried over anh Na₂SO₄, filtered and concentrated under vacuum. The residue was purified by column chromatography (SiO₂, petroleum ether) and then reversed phase flash chromatography [water (FA, 0.1%)/acetonitrile] to afford triisopropyl((8-(trimethylstannyl)naphthalen-1-yl)ethynyl)silane (0.65 g, 1.37 mmol, 8.1% yield) as a colorless oil. ¹H NMR (400 MHz, chloroform-d): 6=7.90 (dd, J=1.2, 7.2 Hz, 1H), 7.86-7.79 (m, 3H), 7.47-7.39 (m, 2H), 1.25-1.18 (m, 21H), 0.54-0.44 (m, 9H).

### Intermediate B-7

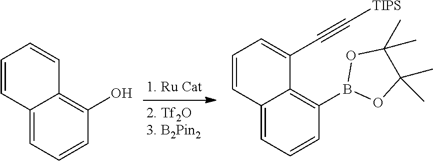

To a solution of naphthalen-1-ol (500 mg, 3.47 mmol, 1.00 equiv), potassium carbonate (479 mg, 3.47 mmol, 1.00 equiv), (p-cymene)ruthenium(II) chloride dimer (531 mg, 867 μmol, 0.25 equiv) and sodium acetate (56.9 mg, 694 μmol, 0.20 eq) in DCE (20.0 mL) was added 2-bromoethynyl(triisopropyl)silane (1.09 g, 4.16 mmol, 1.20 equiv) and the reaction was stirred at 40° C. for 12 h. The reaction was cooled to 25° C., filtered and concentrated at reduced pressure. The residue was purified by silica gel chromatography (petroleum ether/ethyl acetate, 1:0 to 10:1) to give 8-((triisopropylsilyl)ethynyl)naphthalen-1-ol (760 mg, 67% yield) as a brown solid. ¹H NMR (400 MHz, CDCl₃): δ 9.22 (s, 1H), 7.81 (dd, J=1.2, 8.4 Hz, 1H), 7.64 (dd, J=1.2, 6.8 Hz, 1H), 7.42-7.34 (m, 3H), 7.01 (dd, J=4.0, 5.6 Hz, 1H), 1.25-1.13 (m, 21H).

To a solution of 8-((triisopropylsilyl)ethynyl)naphthalen-1-ol (760 mg, 2.34 mmol, 1.00 equiv) and DIEA (816 μL, 4.68 mmol, 2.00 equiv) in DCM (8.00 mL) was added Tf₂O (580 μL, 3.51 mmol, 1.50 equiv) at −40° C. The reaction was stirred at 25° C. for 0.5 h prior to being diluted with water (10.0 mL). The aqueous phase was extracted with DCM (2×20.0 mL). The combined organic phase was washed with brine (2×20.0 mL), dried with anhydrous sodium sulfate, filtered and concentrated. The residue was purified by silica gel chromatography (petroleum ether/ethyl acetate, 1:0 to 10:1) to give 8-((triisopropylsilyl)ethynyl)naphthalen-1-yl trifluoromethanesulfonate (1.00 g, 93% yield) as a brown oil. ¹H NMR (400 MHz, CDCl₃): δ 7.92-7.84 (m, 3H), 7.56-7.47 (m, 3H), 1.26-1.12 (m, 21H).

To a solution of 8-((triisopropylsilyl)ethynyl)naphthalen-1-yl trifluoromethanesulfonate (950 mg, 2.08 mmol, 1.00 equiv) in dioxane (15.0 mL) was added bis-pinacolatodiboron (687 mg, 2.70 mmol, 1.30 equiv), Pd(dppf)Cl₂ (152 mg, 208 μmol, 0.10 equiv) and KOAc (408 mg, 4.16 mmol, 2.00 equiv). The reaction mixture was stirred at 110° C. for 5 hours under nitrogen. The mixture was filtered and concentrated under reduced pressure. The residue was purified by silica gel chromatography (petroleum ether/ethyl acetate, 1:0 to 20:1) to give triisopropyl((8-(4,4,5,5-tetramethyl-1,3,2-dioxaborolan-2-yl)naphthalen-1-yl)ethynyl)silane (340 mg, 38% yield) as a red oil. ¹H NMR (400 MHz, CDCl₃): δ 7.78-7.66 (m, 3H), 7.39-7.26 (m, 3H), 1.36 (s, 12H), 1.12-1.05 (m, 21H).

### Intermediate C-1

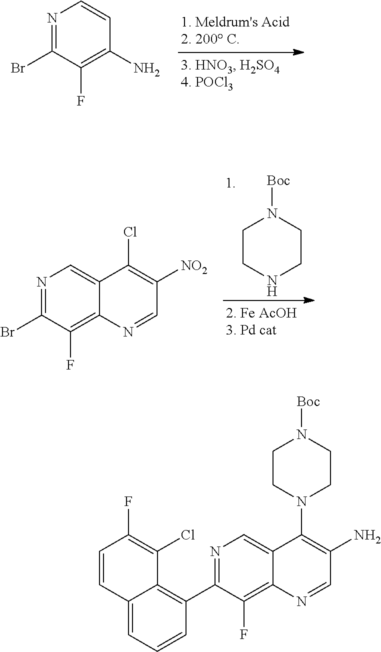 [SMILES: Nc1ccnc(Br)c1F ; O=[N+]([O-])c1cnc2c(F)c(Br)ncc2c1Cl ; CC(C)(C)OC(=O)N1CCNCC1 ; CC(C)(C)OC(=O)N1CCN(c2c(N)cnc3c(F)c(-c4cccc5ccc(F)c(Cl)c45)ncc23)CC1]

To a solution of Meldrum's acid (14.1 g, 75.8 mmol, 1 equiv) in trimethyl orthoformate (100 mL) was added DIEA (19.5 g, 151 mmol, 26.4 mL, 2 equiv), 2-bromo-3-fluoropyridin-4-amine (20.0 g, 75.8 mmol, 1 equiv, 2HCl). The mixture was stirred at 85° C. for 1 hour. The mixture was cooled to rt and was washed with isopropanol (50.0 mL) and pentane (3×100 mL). The solid was filtered to afford 5-(((2-bromo-3-fluoropyridin-4-yl)amino)methylene)-2,2-dimethyl-1,3-dioxane-4,6-dione (64.0 g, 185 mmol, 81% yield) as a white solid. ¹H NMR (400 MHz, DMSO-d₆): δ 11.43-11.23 (m, 1H), 8.90-8.75 (m, 1H), 8.32-8.17 (m, 1H), 8.06-7.94 (m, 1H), 1.70 (s, 6H).

A solution of 5-(((2-bromo-3-fluoropyridin-4-yl)amino)methylene)-2,2-dimethyl-1,3-dioxane-4,6-dione (20.0 g, 57.9 mmol, 1 equiv) in the diphenylether (400 mL) was stirred at 200° C. for 1 hour. The mixture was cooled to room temperature and diluted with petroleum ether (1 L). The resultant solid was filtered and dried under the reduced pressure to afford 7-bromo-8-fluoro-1,6-naphthyridin-4-ol (10.0 g, 41.1 mmol, 71% yield) as a brown solid. LCMS [ESI, M+1]: 243.0, 245.0.

To a solution of 7-bromo-8-fluoro-1,6-naphthyridin-4-ol (13.0 g, 53.5 mmol, 1 equiv) in ice-cold H₂SO₄ (100 mL) was added KNO₃ (10.8 g, 106 mmol, 2 equiv) slowly at 0° C. The mixture was stirred at 100° C. for 1 hour prior to being cooled to room temperature. The mixture was poured into ice water (600 mL) and was filtered. The filter cake was washed by ACN (20.0 mL) to afford 7-bromo-8-fluoro-3-nitro-1,6-naphthyridin-4-ol (4.30 g, 14.0 mmol, 26% yield) as a yellow solid. LCMS [ESI, M+1]: 287.9, 289.9.

To a solution of 7-bromo-8-fluoro-3-nitro-1,6-naphthyridin-4-ol (4.30 g, 14.9 mmol, 1 equiv) in DCE (50.0 mL) was added DIEA (5.79 g, 44.7 mmol, 7.80 mL, 3 equiv) and POCl₃ (22.8 g, 149.2 mmol, 13.8 mL, 10 equiv) at 20° C. The mixture was stirred at 70° C. for 2 hours and was concentrated under reduced pressure at 50° C. to dryness to provide 7-bromo-4-chloro-8-fluoro-3-nitro-1,6-naphthyridine (5 g, crude) as a brown oil.

To a solution of 7-bromo-4-chloro-8-fluoro-3-nitro-1,6-naphthyridine (5 g, 16.3 mmol, 1 equiv) in DCM (50.0 mL) was added DIEA (21.0 g, 163 mmol, 28.4 mL, 10 equiv) and tert-butyl piperazine-1-carboxylate (6.08 g, 32.6 mmol, 2 equiv). The mixture was stirred at 20° C. for 1 hour. The mixture was diluted with saturated NaHCO₃ (200 mL) and extracted with ethyl acetate (3×100 mL). The combined organic layer was washed with brine (100 mL), dried over anh Na₂SO₄, filtered and concentrated under reduced pressure to give a residue. The residue was purified by column chromatography (SiO₂, petroleum ether/ethyl acetate, 3:1) to afford tert-butyl 4-(7-bromo-8-fluoro-3-nitro-1,6-naphthyridin-4-yl)piperazine-1-carboxylate (4.60 g, 10.0 mmol, 61% yield) as a yellow solid. LCMS [ESI, M+1]: 456.1, 458.1.

To a solution of tert-butyl 4-(7-bromo-8-fluoro-3-nitro-1,6-naphthyridin-4-yl)piperazine-1-carboxylate (2.00 g, 4.38 mmol, 1 equiv) in AcOH (20.0 mL) was added Fe powder (1.22 g, 21.9 mmol, 5 equiv) in portions. The mixture was stirred at 60° C. for 1 hour and was filtered. The filtrate was diluted with saturated NaHCO₃ (200 mL) and extracted with ethyl acetate (50.0 mL). The aqueous phase was further diluted with saturated NaHCO₃ (100 mL) and extracted with EtOAc (3×10.0 mL). The combined organic phase was washed with brine (3×10.0 mL), dried over anh Na₂SO₄, filtered and concentrated under reduced pressure to afford tert-butyl 4-(3-amino-7-bromo-8-fluoro-1,6-naphthyridin-4-yl)piperazine-1-carboxylate (1.80 g, 4.22 mmol, 96% yield) as a yellow solid. LCMS [ESI, M+1]: 426.1, 428.1.

To a solution of tert-butyl 4-(3-amino-7-bromo-8-fluoro-1,6-naphthyridin-4-yl)piperazine-1-carboxylate (1.00 g, 2.35 mmol, 1 equiv) and 2-(8-chloro-7-fluoronaphthalen-1-yl)-4,4,5,5-tetramethyl-1,3,2-dioxaborolane (755 mg, 2.46 mmol, 1.05 equiv) in dioxane (30.0 mL) and H₂O (6.00 mL) was added Pd₂(dba)₃ (134 mg, 234 μmol, 0.1 equiv), bis(di-tert-butyl)-4-dimethylaminophenylphosphine (124 mg, 469 μmol, 0.2 equiv) and K₃PO₄ (1.49 g, 7.04 mmol, 3 equiv). The vessel was purged with nitrogen and stirred at 70° C. for 10 hours. The mixture was diluted with water (10.0 mL) and extracted with ethyl acetate (3×10.0 mL). The combined organic layer was washed with brine (3×10.0 mL), dried over anh Na₂SO₄, filtered and concentrated under reduced pressure to give a residue. The residue was triturated with ethyl acetate (10 mL) and the solid was filtered to provide tert-butyl 4-(3-amino-7-(8-chloro-7-fluoronaphthalen-1-yl)-8-fluoro-1,6-naphthyridin-4-yl)piperazine-1-carboxylate (800 mg, 1.52 mmol, 64% yield) as a yellow solid. LCMS [ESI, M+1]: 526.3.

### Intermediate C-2

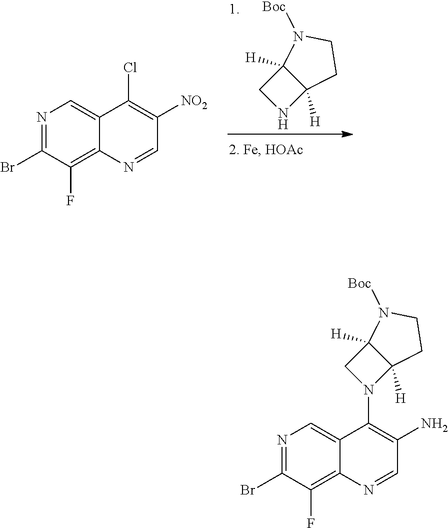 [SMILES: O=[N+]([O-])c1cnc2c(F)c(Br)ncc2c1Cl ; CC(C)(C)OC(=O)N1CC[C@H]2NC[C@H]21 ; CC(C)(C)OC(=O)N1CC[C@@H]2[C@H]1CN2c1c(N)cnc2c(F)c(Br)ncc12]

To a solution of 7-bromo-4-chloro-8-fluoro-3-nitro-1,6-naphthyridine (500 mg, 1.6 mmol, 1.0 equiv) in DCM (5 mL) was added DIEA (2.1 g, 16.3 mmol, 2.8 mL, 10 equiv) and tert-butyl (1R,5R)-4,7-diazabicyclo[3.2.0]heptane-4-carboxylate (323 mg, 1.63 mmol, 1.0 equiv) at 0° C. The mixture was stirred at 20° C. for 1 hour. The mixture was diluted with saturated NaHCO₃ aqueous (30 mL) and extracted with EtOAc (3×10 mL). The combined organic layer was washed with brine (30 mL), dried over anh Na₂SO₄, filtered and concentrated under reduced pressure to give a residue. The residue was purified by prep-TLC (SiO₂, petroleum ether/ethyl acetate, 3:1) to afford tert-butyl (1R,5R)-6-(7-bromo-8-fluoro-3-nitro-1,6-naphthyridin-4-yl)-2,6-diazabicyclo[3.2.0]heptane-2-carboxylate (450 mg, 961 μmol, 59% yield) as a red solid.

To a solution of tert-butyl (1R,5R)-6-(7-bromo-8-fluoro-3-nitro-1,6-naphthyridin-4-yl)-2,6-diazabicyclo[3.2.0]heptane-2-carboxylaten (450 mg, 960.9 μmol, 1 equiv) in AcOH (5 mL) was added Fe powder (268 mg, 4.8 mmol, 5 equiv). The mixture was stirred at 60° C. for 1 hour. The reaction was diluted with satd aq NaHCO₃ (30 mL) and extracted with EtOAc (3×10 mL). The combined organic layer was washed with brine (10 mL), dried over anh Na₂SO₄, filtered and concentrated under reduced pressure to give a residue. The residue was purified by prep-TLC (SiO₂, petroleum ether/ethyl acetate, 1:1) to provide tert-butyl (1R,5R)-6-(3-amino-7-bromo-8-fluoro-1,6-naphthyridin-4-yl)-2,6-diazabicyclo[3.2.0]heptane-2-carboxylate (240 mg, 547 μmol, 57% yield) as a yellow solid. LCMS [ESI, M+1]: 438.1, 440.1.

### Intermediate D-1

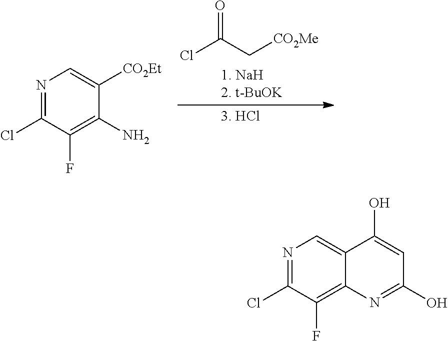 [SMILES: COC(=O)CC(=O)Cl ; CCOC(=O)c1cnc(Cl)c(F)c1N ; Oc1cc(O)c2cnc(Cl)c(F)c2n1]

To a mixture of ethyl 4-amino-6-chloro-5-fluoronicotinate (0.50 g, 2.29 mmol, 1.0 equiv) in THE (10 mL) was added NaH (274 mg, 6.86 mmol, 60% purity, 3.0 equiv) at 0° C. The mixture was stirred at 0° C. for 0.5 h prior to the addition of methyl 3-chloro-3-oxo-propanoate (625 mg, 4.57 mmol, 488 μL, 2.0 equiv). The resultant mixture was stirred at 0° C. for an additional 0.5 h. The mixture was poured into ice water (10 mL) and extracted with ethyl acetate (2×10 mL). The combined organic layer was washed with brine (10 mL), dried over anh Na₂SO₄, filtered and concentrated under vacuum. The residue was purified by reversed phase flash chromatography [water (FA, 0.1%)/acetonitrile] to give ethyl 6-chloro-5-fluoro-4-(3-methoxy-3-oxopropanamido)nicotinate (150 mg, 16% yield) as a yellow solid. LCMS [ESI, M+1]: 319.0.

To a solution of ethyl 6-chloro-5-fluoro-4-(3-methoxy-3-oxopropanamido)nicotinate (6.20 g, 19.5 mmol, 1.0 equiv) in THE (200 mL) was added t-BuOK (1.0 M in THF, 58.4 mL, 3.0 equiv) at 0° C. The mixture was stirred at 20° C. for 0.5 h prior to being concentrated under vacuum. The residue was diluted with water (100 mL) and the pH as adjusted to 6 with aq HCl (2.0 M). The solid was filtered and dried under vacuum to provide ethyl 7-chloro-8-fluoro-2,4-dihydroxy-1,6-naphthyridine-3-carboxylate (5.00 g, 94% yield) as a white solid.

A mixture of ethyl 7-chloro-8-fluoro-2,4-dihydroxy-1,6-naphthyridine-3-carboxylate (5.30 g, 19.4 mmol, 1.0 equiv) in HCl (12.0 M, 50 mL, 30.9 equiv) was stirred at 90° C. for 2 h. Subsequently, the mixture was concentrated under vacuum and the resultant residue was diluted with H₂O (100 mL) and filtered. The solid was dried under vacuum to give 7-chloro-8-fluoro-1,6-naphthyridine-2,4-diol (3 g, 60% yield) as a yellow solid. LCMS [ESI, M+1]: 215.0.

### Example 1

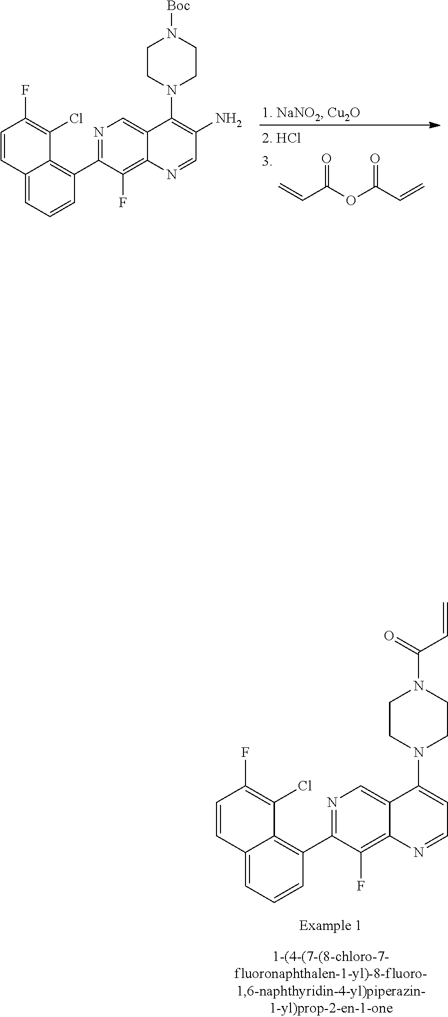

To a solution of tert-butyl 4-(3-amino-7-(8-chloro-7-fluoronaphthalen-1-yl)-8-fluoro-1,6-naphthyridin-4-yl)piperazine-1-carboxylate (150 mg, 285 μmol, 1 equiv) in EtOH (3.00 mL), H₂SO₄ (0.2 mL) and H₂O (0.2 mL) was added NaNO₂ (21.6 mg, 313 μmol, 1.1 equiv), Cu₂O (81.6 mg, 570 μmol, 2 equiv) at 20° C. The mixture was stirred at 80° C. for 2 hours. The mixture was diluted with saturated NaHCO₃ (100 mL) and extracted with EA (3×50.0 mL). The combined organic layer was washed with brine (50 mL), dried over anh Na₂SO₄, filtered and concentrated under reduced pressure to give a residue. The residue was purified by prep-TLC (DCM/MeOH, 10:1) to afford tert-butyl 4-(7-(8-chloro-7-fluoronaphthalen-1-yl)-8-fluoro-1,6-naphthyridin-4-yl)piperazine-1-carboxylate (50 mg, 97.8 μmol, 34% yield) as a yellow solid. LCMS [ESI, M+1]: 511.2.

To a solution of tert-butyl 4-(7-(8-chloro-7-fluoronaphthalen-1-yl)-8-fluoro-1,6-naphthyridin-4-yl)piperazine-1-carboxylate (40.0 mg, 97.8 μmol, 1 equiv) in EtOAc (0.1 mL) was added HCl·EtOAc (4 M, 3.00 mL). The mixture was stirred at 25° C. for 1 hour. The reaction mixture was concentrated under reduced pressure to provide 7-(8-chloro-7-fluoronaphthalen-1-yl)-8-fluoro-4-(piperazin-1-yl)-1,6-naphthyridine (40.0 mg, crude) as a yellow solid. LCMS [ESI, M+1]: 411.1.

To a solution of 7-(8-chloro-7-fluoronaphthalen-1-yl)-8-fluoro-4-(piperazin-1-yl)-1,6-naphthyridine (40.0 mg, 97.3 μmol, 1 equiv) in DCM (1.00 mL) was added TEA (49.2 mg, 486 μmol, 67.7 μL, 5 equiv) and prop-2-enoyl prop-2-enoate (24.5 mg, 195 μmol, 1.5 equiv) at −40° C. The mixture was stirred at −40° C. for 1 hour. Subsequently, the reaction mixture was diluted with water (10.0 mL) and extracted with DCM (3×10.0 mL). The combined organic layer was washed with brine (3×10.0 mL), dried over anh Na₂SO₄, filtered and concentrated under reduced pressure to give a residue. The residue was purified by prep-HPLC [column: Waters Xbridge Prep OBD C18 100×25 mm×5 μm; mobile phase: water (10 mM NH₄HCO₃), B: ACN, B %: 30%-60%; min) to afford 1-(4-(7-(8-chloro-7-fluoronaphthalen-1-yl)-8-fluoro-1,6-naphthyridin-4-yl)piperazin-1-yl)prop-2-en-1-one (7.25 mg, 98.0% purity) as a white solid. ¹H NMR (400 MHz, CDCl₃): δ 9.32 (s, 1H), 9.03-8.95 (m, 1H), 8.02 (d, J=8.0 Hz, 1H), 7.91 (dd, J=5.2, 8.8 Hz, 1H), 7.71-7.66 (m, 1H), 7.65-7.59 (m, 1H), 7.41 (t, J=8.4 Hz, 1H), 6.99 (d, J=4.8 Hz, 1H), 6.71-6.60 (m, 1H), 6.43-6.36 (m, 1H), 5.81 (dd, J=1.6, 10.4 Hz, 1H), 4.18-3.84 (m, 4H), 3.56-3.39 (m, 4H); LCMS [ESI, M+1]: 465.1.

### Example 2

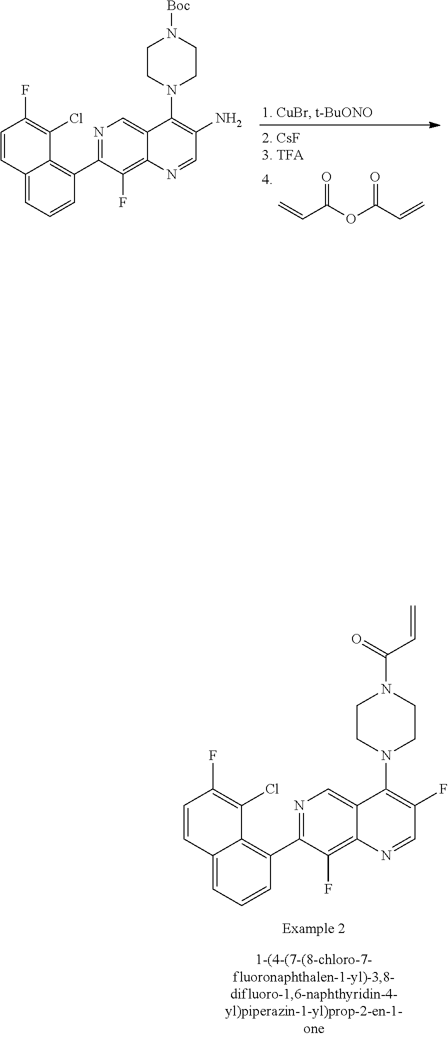

To a suspension of CuBr (109 mg, 760 μmol, 2 equiv) in MeCN (6.00 mL) was added tert-butylnitrite (52.9 mg, 513 μmol, 61.0 μL, 1.35 equiv). The mixture was flushed with nitrogen and stirred at 65° C. for 1 hour. To this solution was added tert-butyl 4-(3-amino-7-(8-chloro-7-fluoronaphthalen-1-yl)-8-fluoro-1,6-naphthyridin-4-yl)piperazine-1-carboxylate (200 mg, 380 μmol, 1 equiv) was added, the mixture was stirred at 65° C. for 3 hours. The reaction mixture was filtered and the filtrate was diluted with EtOAc (10 mL). The organic layer was washed with brine (3×10 mL), dried over anh Na₂SO₄, filtered and concentrated under reduced pressure to give a residue. The residue was purified by prep-TLC (SiO₂, petroleum ether/ethyl acetate, 1:1) to give tert-butyl 4-(3-bromo-7-(8-chloro-7-fluoronaphthalen-1-yl)-8-fluoro-1,6-naphthyridin-4-yl)piperazine-1-carboxylate (120 mg, 203 μmol, 53% yield) as a yellow solid. LCMS [ESI, M+1]: 589.2, 591.2.

To a solution of tert-butyl 4-(3-bromo-7-(8-chloro-7-fluoronaphthalen-1-yl)-8-fluoro-1,6-naphthyridin-4-yl)piperazine-1-carboxylate (120 mg, 203 μmol, 1 equiv) in DMAc (3.00 mL) was added CsF (155 mg, 1.02 mmol, 5 equiv). The mixture was stirred at 130° C. for 48 hours. The mixture was cooled to room temperature and diluted with H₂O (50 mL) and extracted with EtOAc (3×10 mL). The combined organic layer was washed with brine (10 mL), dried over anh Na₂SO₄, filtered and concentrated under reduced pressure to give a residue. The residue was purified by prep-TLC (petroleum ether/ethyl acetate, 1:1) to provide tert-butyl 4-(7-(8-chloro-7-fluoronaphthalen-1-yl)-3,8-difluoro-1,6-naphthyridin-4-yl)piperazine-1-carboxylate (30.0 mg, 56.7 μmol, 28% yield) as a white solid. LCMS [ESI, M+1]: 529.2.

To a solution of tert-butyl 4-(7-(8-chloro-7-fluoronaphthalen-1-yl)-3,8-difluoro-1,6-naphthyridin-4-yl)piperazine-1-carboxylate (30.0 mg, 56.7 μmol, 1 equiv) in DCM (3.00 mL) was added TFA (924 mg, 8.10 mmol, 0.60 mL). The mixture was stirred at 0° C. for 1 hour. Subsequently, the mixture was diluted with saturated aq NaHCO₃ (20 mL) and extracted with DCM (3×10 mL). The combined organic layer was washed with brine (3×10 mL), dried over anh Na₂SO₄, filtered and concentrated under reduced pressure to afford 7-(8-chloro-7-fluoronaphthalen-1-yl)-3,8-difluoro-4-(piperazin-1-yl)-1,6-naphthyridine (20.0 mg, crude) as a yellow solid.

To a solution of 7-(8-chloro-7-fluoronaphthalen-1-yl)-3,8-difluoro-4-(piperazin-1-yl)-1,6-naphthyridine (30.0 mg, 1 equiv) in DCM (1.00 mL) was added TEA (14.2 mg, 140 μmol, 19.5 μL, 3 equiv) and prop-2-enoyl prop-2-enoate (8.82 mg, 69.9 umol, 1.5 eq) at −40° C. The mixture was stirred at −40° C. for 1 hour. Subsequently, the reaction mixture was diluted with water (10 mL) and extracted with DCM (3×10 mL). The combined organic layer was washed with brine (3×10 mL), dried over anh Na₂SO₄, filtered and concentrated under reduced pressure to give a residue. The residue was purified by prep-HPLC [column: Waters Xbridge BEH C18 100*25 mm*Sum; mobile phase: A: water (10 mM NH₄HCO₃), B: ACN, B %: 35%-65%; 10 min] to give 1-(4-(7-(8-chloro-7-fluoronaphthalen-1-yl)-3,8-difluoro-1,6-naphthyridin-4-yl)piperazin-1-yl)prop-2-en-1-one (9.50 mg, 19.3 μmol, 41% yield over two steps, 98.2% purity) as a white solid. ¹H NMR (400 MHz, CDCl₃): δ=9.35 (s, 1H), 8.92 (d, J=4.4 Hz, 1H), 8.02 (dd, J=1.6, 8.0 Hz, 1H), 7.92 (dd, J=5.6, 9.2 Hz, 1H), 7.66-7.60 (m, 2H), 7.41 (t, J=8.8 Hz, 1H), 6.64 (dd, J=10.8, 16.8 Hz, 1H), 6.40 (dd, J=2.0, 16.8 Hz, 1H), 5.80 (dd, J=1.6, 10.4 Hz, 1H), 4.02-3.87 (m, 4H), 3.61-3.60 (m, 4H). LCMS [ESI, M+1]: 483.1.

### Example 3

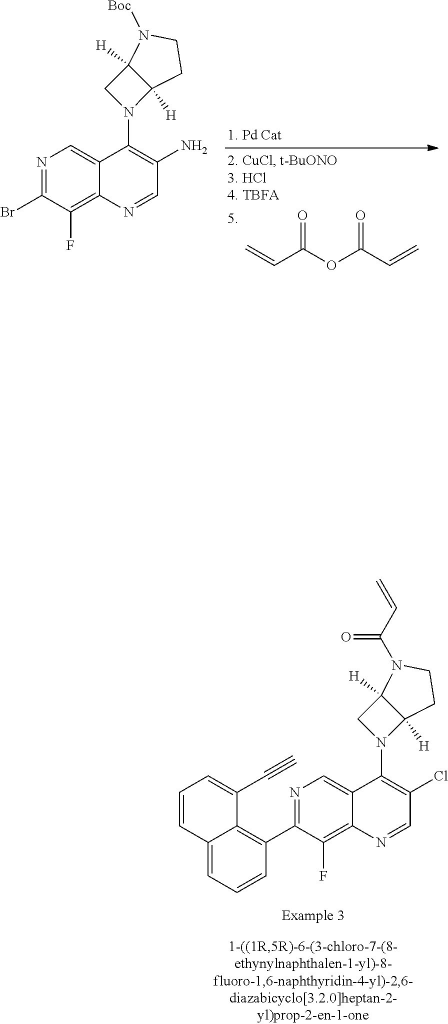

To a solution of tert-butyl (1R,5R)-6-(3-amino-7-bromo-8-fluoro-1,6-naphthyridin-4-yl)-2,6-diazabicyclo[3.2.0]heptane-2-carboxylate (200 mg, 456.3 μmol, 1 equiv) and triisopropyl((8-(4,4,5,5-tetramethyl-1,3,2-dioxaborolan-2-yl)naphthalen-1-yl)ethynyl)silane (198 mg, 456 μmol, 1 equiv) in THE (4 mL) was added K₃PO₄ (1.5 M, 1.2 mL, 4 equiv) and Xphos Pd G3 (66.5 mg, 91.3 μmol, 0.2 equiv). The mixture was stirred at 60° C. for 16 hours. Subsequently, the reaction mixture was diluted with H₂O (30 mL) and extracted with EtOAc (3×10 mL). The combined organic layer was washed with brine (10 mL), dried over anh Na₂SO₄, filtered and concentrated under reduced pressure to give a residue. The residue was purified by prep-TLC (SiO₂, ethyl acetate) to afford tert-butyl (1R,5R)-6-(3-amino-8-fluoro-7-(8-((triisopropylsilyl)ethynyl)naphthalen-1-yl)-1,6-naphthyridin-4-yl)-2,6-diazabicyclo[3.2.0]heptane-2-carboxylate (180 mg, 270 μmol, 59% yield) as a yellow oil. LCMS [ESI, M+1]: 666.3.

To a mixture of CuCl₂ (60.6 mg, 450 μmol, 2 equiv), t-BuONO (34.8 mg, 338 μmol, 40.2 μL, 1.5 equiv) and CuCl (44.6 mg, 450 μmol, 2 equiv) in MeCN (2 mL) was added tert-butyl (1R,5R)-6-(3-amino-8-fluoro-7-(8-((triisopropylsilyl)ethynyl)naphthalen-1-yl)-1,6-naphthyridin-4-yl)-2,6-diazabicyclo[3.2.0]heptane-2-carboxylate (150 mg, 225 μmol, 1 equiv). The mixture was stirred at 60° C. for 2 hours. The mixture was cooled to room temperature and poured into H₂O (8 mL) and extracted with EtOAc (3×4 mL). The combined organic layer was washed with brine (10 mL), dried over anh Na₂SO₄, filtered and concentrated under reduced pressure. The crude material was purified by column chromatography (petroleum ether/ethyl acetate, 40:1 to 10:1) to give tert-butyl (1R,5R)-6-(3-chloro-8-fluoro-7-(8-((triisopropylsilyl)ethynyl)naphthalen-1-yl)-1,6-naphthyridin-4-yl)-2,6-diazabicyclo[3.2.0]heptane-2-carboxylate (80 mg, 52% yield) as a brown oil. ¹H NMR (400 MHz, chloroform-d): δ 9.26 (d, J=9.2 Hz, 1H), 8.61 (s, 1H), 8.00-7.91 (m, 2H), 7.81 (d, J=6.8 Hz, 1H), 7.62-7.56 (m, 2H), 7.47 (t, J=7.6 Hz, 1H), 6.00-5.79 (m, 1H), 4.88-4.51 (m, 4H), 4.06-3.59 (m, 3H), 2.65-2.16 (m, 3H), 1.54-1.47 (m, 12H), 0.91-0.81 (m, 20H); LCMS [ESI, M+1]: 685.

A mixture of tert-butyl (1R,5R)-6-(3-chloro-8-fluoro-7-(8-((triisopropylsilyl)ethynyl)naphthalen-1-yl)-1,6-naphthyridin-4-yl)-2,6-diazabicyclo[3.2.0]heptane-2-carboxylate (80 mg, 117 μmol, 1 equiv) in HCl·EtOAc (4 M, 2 mL) was stirred at 25° C. for 2 hours. The mixture was concentrated under reduced pressure to provide 4-((1R,5R)-2,6-diazabicyclo[3.2.0]heptan-6-yl)-3-chloro-8-fluoro-7-(8-((triisopropylsilyl)ethynyl)naphthalen-1-yl)-1,6-naphthyridine (60 mg, crude) as a brown oil. LCMS [ESI, M+1]: 585.

A mixture of 4-((1R,5R)-2,6-diazabicyclo[3.2.0]heptan-6-yl)-3-chloro-8-fluoro-7-(8-((triisopropylsilyl)ethynyl)naphthalen-1-yl)-1,6-naphthyridine (60 mg, 102.52 μmol, 1 equiv) in TBAF (1 M, 1 mL) was stirred at 25° C. for 10 minutes. The mixture was concentrated under reduced pressure to give 4-((1R,5R)-2,6-diazabicyclo[3.2.0]heptan-6-yl)-3-chloro-7-(8-ethynylnaphthalen-1-yl)-8-fluoro-1,6-naphthyridine (45 mg, crude) as a brown oil. LCMS [ESI, M+1]: 429.

To a mixture of prop-2-enoyl prop-2-enoate (23.5 mg, 186 μmol, 2 equiv) and TEA (28.3 mg, 280 μmol, 3.0 equiv) in DCM (2 mL) was added 4-((1R,5R)-2,6-diazabicyclo[3.2.0]heptan-6-yl)-3-chloro-7-(8-ethynylnaphthalen-1-yl)-8-fluoro-1,6-naphthyridine (40 mg, 93.26 μmol, 1 equiv). The mixture was stirred at −40° C. for 10 minutes prior to being poured into H₂O (10 mL) and extracted with DCM (4 mL). The combined organic layer was washed with brine (5 mL), dried over anh Na₂SO₄, filtered and concentrated under reduced pressure. The crude material was purified by prep-HPLC (column: Waters Xbridge BEH C18 100*25 mm*5 um; mobile phase: A: water (10 mM NH₄HCO₃), B: ACN, B %: 30%-60%, 10 min) to afford 1-((1R,5R)-6-(3-chloro-7-(8-ethynylnaphthalen-1-yl)-8-fluoro-1,6-naphthyridin-4-yl)-2,6-diazabicyclo[3.2.0]heptan-2-yl)prop-2-en-1-one (4.9 mg, 11% yield over three steps) as a colorless oil. ¹H NMR (400 MHz, chloroform-d): δ 9.27-9.12 (m, 1H), 8.71-8.56 (m, 1H), 8.09-7.92 (m, 2H), 7.76 (br d, J=7.2 Hz, 1H), 7.68-7.59 (m, 2H), 7.53-7.40 (m, 1H), 6.65-6.26 (m, 2H), 6.11-5.89 (m, 1H), 5.85-5.74 (m, 1H), 5.18-4.68 (m, 3H), 4.47-4.10 (m, 1H), 4.08-3.75 (m, 1H), 2.83-2.57 (m, 1H), 2.51-2.22 (m, 2H). LCMS [ESI, M+1]: 483.

### Example 4

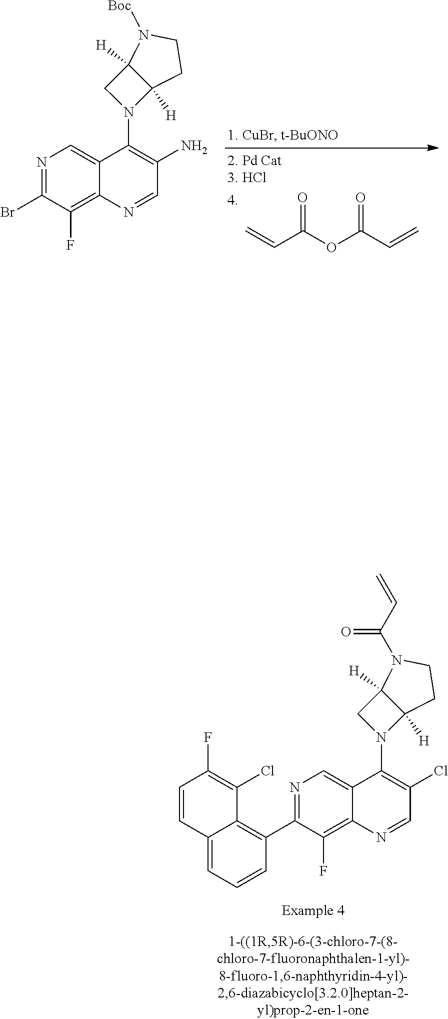

To a solution of (1R,5R)-tert-butyl 6-(3-amino-7-bromo-8-fluoro-1,6-naphthyridin-4-yl)-2,6-diazabicyclo[3.2.0]heptane-2-carboxylate (500 mg, 1.14 mmol, 1.00 equiv) in CH₃CN (10.0 mL) was added CuCl (2.3 g, 2.28 mmol, 2.00 equiv) and CuCl₂ (461 mg, 3.42 mmol, 3.00 equiv) with stirring at 15° C. After 10 min, isopentyl nitrite (267.8 mg, 2.28 mmol, 307 μL, 2 equiv) in CH₃CN (1.00 mL) was added and the mixture was stirred at 15° C. for 4 h under nitrogen. The reaction mixture was diluted with ice cold water (20.0 mL) and extracted with EtOAc (3×20.0 mL). The combined organic layer was washed with brine (20.0 mL), dried over anh Na₂SO₄, filtered and concentrated under reduced pressure. The residue was purified by flash silica gel chromatography (petroleum ether:ethyl acetate, 0:100 to 1:2) to afford tert-butyl (1R,5R)-6-(7-bromo-3-chloro-8-fluoro-1,6-naphthyridin-4-yl)-2,6-diazabicyclo[3.2.0]heptane-2-carboxylate (300 mg, 58% yield) as a yellow syrup. LCMS [M+1]: 459.1.

To a solution of tert-butyl (1R,5R)-6-(7-bromo-3-chloro-8-fluoro-1,6-naphthyridin-4-yl)-2,6-diazabicyclo[3.2.0]heptane-2-carboxylate (200 mg, 437 μmol, 1.00 equiv) and (8-chloro-7-fluoronaphthalen-1-yl)trimethylstannane (151 mg, 437 μmol, 1.00 equiv) in DMF (6.00 mL) was added Pd(dppf)Cl₂—CH₂Cl₂ (35.7 mg, 43.7 μmol, 0.10 equiv) under N₂. The mixture was stirred at 100° C. for 16 h. The reaction mixture was cooled to room temperature and diluted with ice cold water (20.0 mL) and extracted with EtOAc (3×20.0 mL). The combined organic layer was washed with brine (20.0 mL), dried over anh Na₂SO₄, filtered and concentrated under reduced pressure. The crude residue was purified by flash silica gel chromatography (petroleum ether/ethyl acetate, 100:1 to 0:1) to provide tert-butyl (1R,5R)-6-(3-chloro-7-(8-chloro-7-fluoronaphthalen-1-yl)-8-fluoro-1,6-naphthyridin-4-yl)-2,6-diazabicyclo[3.2.0]heptane-2-carboxylate (40.0 mg, 16% yield) as a yellow solid. LCMS [M+1]: 557.2.

To a solution of tert-butyl (1R,5R)-6-(3-chloro-7-(8-chloro-7-fluoronaphthalen-1-yl)-8-fluoro-1,6-naphthyridin-4-yl)-2,6-diazabicyclo[3.2.0]heptane-2-carboxylate (30.0 mg, 53.8 μmol, 1.00 equiv) in EtOAc (2.00 mL) was added HCl-EtOAc (2.00 mL, 4 M) under N₂. The mixture was stirred at 20° C. for 2 h prior to being concentrated under reduced pressure to give 4-((1R,5R)-2,6-diazabicyclo[3.2.0]heptan-6-yl)-3-chloro-7-(8-chloro-7-fluoronaphthalen-1-yl)-8-fluoro-1,6-naphthyridine (30.0 mg, 100% yield) as a yellow solid. LCMS [M+1]: 457.1.

To a solution of 4-((1R,5R)-2,6-diazabicyclo[3.2.0]heptan-6-yl)-3-chloro-7-(8-chloro-7-fluoronaphthalen-1-yl)-8-fluoro-1,6-naphthyridine (30.0 mg) and TEA (18.5 mg, 183 μmol, 25.4 μL, 3.00 eq) in DCM (3.00 mL) was added acrylic anhydride (15.4 mg, 122 μmol, 2.00 equiv). The mixture was stirred at −40° C. for 10 min. The reaction mixture was diluted with ice cold water (5.00 mL) and extracted with DCM (3×5.00 mL). The combined organic layer was washed with brine (5.00 mL), dried over anh Na₂SO₄, filtered and concentrated under reduced pressure. The residue was purified by prep-TLC (SiO₂, EtOAc, twice) to give 1-((1R,5R)-6-(3-chloro-7-(8-chloro-7-fluoronaphthalen-1-yl)-8-fluoro-1,6-naphthyridin-4-yl)-2,6-diazabicyclo[3.2.0]heptan-2-yl)prop-2-en-1-one (7.4 mg, 24% yield) as a white solid. LCMS [M+1]: 511. ¹H NMR (400 MHz, chloroform-d): δ 9.25-9.18 (m, 1H), 8.68-8.62 (m, 1H), 8.01 (br d, J=7.6 Hz, 1H), 7.90 (dd, J=5.6, 8.9 Hz, 1H), 7.74-7.51 (m, 2H), 7.40 (t, J=8.8 Hz, 1H), 6.65-6.29 (m, 2H), 6.18-5.89 (m, 1H), 5.88-5.75 (m, 1H), 5.20-4.82 (m, 2H), 4.80-4.69 (m, 1H), 4.47-4.13 (m, 1H), 4.13-3.82 (m, 1H), 2.75-2.51 (m, 1H), 2.42 (br dd, J=4.4, 10.2 Hz, 1H).

### Example 5

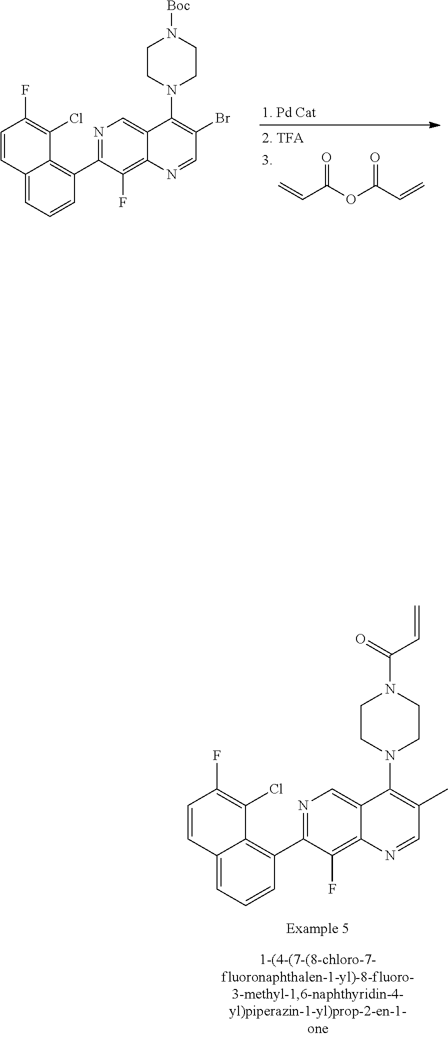

To a solution of tert-butyl 4-(3-bromo-7-(8-chloro-7-fluoronaphthalen-1-yl)-8-fluoro-1,6-naphthyridin-4-yl)piperazine-1-carboxylate (80 mg, 136 μmol, 1.0 equiv) in dioxane (2.0 mL) and H₂O (0.4 mL) was added methyl boronic acid (81.2 mg, 1.36 mmol, 10 equiv), K₂CO₃ (56.2 mg, 407 μmol, 3.0 equiv) and Pd(dppf)Cl₂ (9.92 mg, 13.6 μmol, 0.1 equiv). The mixture was stirred at 80° C. for 3 h. The mixture was diluted with water (2.0 mL) and extracted with ethyl acetate (3×2.0 mL). The combined the organic layer was dried over anh Na₂SO₄, filtered and concentrated. The crude residue was purified by reversed phase flash [water (0.1% FA)/acetonitrile]. The desired fractions were collected and neutralized with solid NaHCO₃ and concentrated under vacuum to remove ACN. The aqueous phase was extracted with ethyl acetate (10 mL×2). The combined organic phase was dried with anhydrous Na₂SO₄, filtered and concentrated in vacuum to give tert-butyl 4-(7-(8-chloro-7-fluoronaphthalen-1-yl)-8-fluoro-3-methyl-1,6-naphthyridin-4-yl)piperazine-1-carboxylate (33 mg, 46%) as a yellow solid. LCMS [ESI, M+1]: 525.1.

To a solution of tert-butyl 4-(7-(8-chloro-7-fluoronaphthalen-1-yl)-8-fluoro-3-methyl-1,6-naphthyridin-4-yl)piperazine-1-carboxylate (27 mg, 51.4 μmol, 1.0 equiv) in DCM (1.0 mL) was added TFA (770 mg, 6.75 mmol, 0.5 mL, 131 equiv) at 0° C. Then the mixture was stirred at 25° C. for 0.5 h. The reaction mixture was concentrated under vacuum to give 7-(8-chloro-7-fluoronaphthalen-1-yl)-8-fluoro-3-methyl-4-(piperazin-1-yl)-1,6-naphthyridine (27 mg, crude, TFA salt) as a white solid.

To a solution of 7-(8-chloro-7-fluoronaphthalen-1-yl)-8-fluoro-3-methyl-4-(piperazin-1-yl)-1,6-naphthyridine (27 mg, 50.1 μmol, 1.0 equiv, TFA salt) in dichloromethane (0.5 mL) was added DIEA (15.1 mg, 117 μmol, 20.4 μL, 2.33 equiv) at −40° C. Subsequently, a solution of prop-2-enoyl prop-2-enoate (5.90 mg, 46.78 μmol, 0.9 equiv) in dichloromethane (0.2 mL) was added dropwise at −40° C. The mixture was stirred at −40° C. for 0.5 hour prior to being diluted with water (1.0 mL) and extracted with ethyl acetate (3×2.0 mL). The combined the organic layers were dried over anh Na₂SO₄, filtered and concentrated. The crude residue was purified by prep-HPLC [column: Waters Xbridge 150×25 mm×5 μm; A: water (10 mM NH₄HCO₃), B: ACN, B %: 42%-72% over 10 min]. The desired fractions were concentrated and the aqueous layer was lyophilized to afford 1-(4-(7-(8-chloro-7-fluoronaphthalen-1-yl)-8-fluoro-3-methyl-1,6-naphthyridin-4-yl)piperazin-1-yl)prop-2-en-1-one (11.8 mg, 49% over two steps) as a white solid. ¹H NMR (400 MHz, CDCl₃): δ 9.45 (s, 1H), 8.89 (s, 1H), 8.01 (dd, J=1.6, 8.0 Hz, 1H), 7.91 (dd, J=5.6, 9.2 Hz, 1H), 7.67-7.59 (m, 2H), 7.41 (t, J=8.8 Hz, 1H), 6.66 (dd, J=10.4, 16.8 Hz, 1H), 6.39 (dd, J=2.0, 16.8 Hz, 1H), 5.80 (dd, J=2.0, 10.4 Hz, 1H), 4.17-3.73 (m, 4H), 3.50 (t, J=4.4 Hz, 4H), 2.58 (s, 3H); LCMS [ESI, M+1]: 479.1.

### Example 6

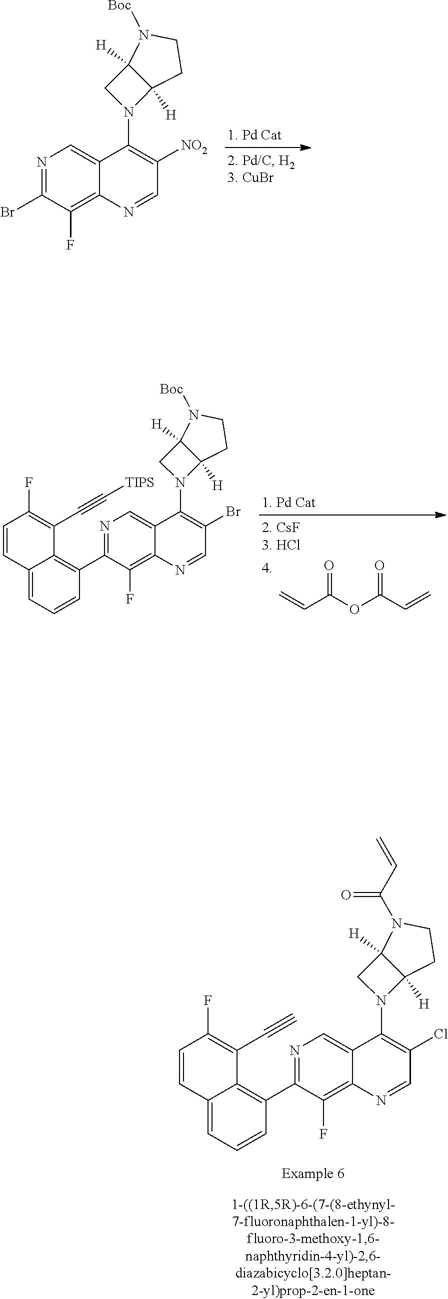

A mixture of tert-butyl (1R,5R)-6-(7-bromo-8-fluoro-3-nitro-1,6-naphthyridin-4-yl)-2,6-diazabicyclo[3.2.0]heptane-2-carboxylate (560 mg, 1.20 mmol, 1.00 equiv), ((2-fluoro-8-(4,4,5,5-tetramethyl-1,3,2-dioxaborolan-2-yl)naphthalen-1-yl)ethynyl)triisopropylsilane (811.67 mg, 1.79 mmol, 1.50 equiv), Ad₂nBuP Pd G3 (cataCXium® A Pd G3) (87.1 mg, 119 μmol, 0.1 equiv), K₃PO₄ (1.50 M, 2.39 mL, 3.00 equiv) in 1,4-dioxane (12.0 mL) was flushed with nitrogen and then the mixture was stirred at 100° C. for 3 h. The reaction mixture was diluted with water (20 mL) and extracted with ethyl acetate (3×30 mL). The combined organic layer was washed with brine (30 mL), dried over anh Na₂SO₄, filtered and concentrated under reduced pressure. The crude residue was purified by reversed phase flash chromatography [water (0.1% formic acid)/acetonitrile)] to afford tert-butyl (1R,5R)-6-(8-fluoro-7-(7-fluoro-8-((triisopropylsilyl)ethynyl)naphthalen-1-yl)-3-nitro-1,6-naphthyridin-4-yl)-2,6-diazabicyclo[3.2.0]heptane-2-carboxylate (824 mg, 96.5% yield) as a yellow solid. LCMS [ESI, M+1]: 714.4.

To a solution of tert-butyl (1R,5R)-6-(8-fluoro-7-(7-fluoro-8-((triisopropylsilyl)ethynyl)naphthalen-1-yl)-3-nitro-1,6-naphthyridin-4-yl)-2,6-diazabicyclo[3.2.0]heptane-2-carboxylate (824 mg, 1.15 mmol, 1.00 equiv) in MeOH (32.0 mL) was added Pd/C (300 mg, 10 wt. %) under N₂. The suspension was stirred under H₂ (15 psi) at 25° C. for 0.5 h. The vessel was flushed with nitrogen and the mixture was filtered and washed with THF (3×5 mL). The filtrate was concentrated under reduced pressure to provide tert-butyl (1R,5R)-6-(3-amino-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (733 mg, 92.9% yield) as a yellow solid. LCMS [ESI, M+1]: 684.4.

To a solution of tert-butyl (1R,5R)-6-(3-amino-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (733 mg, 1.07 mmol, 1.00 equiv) in ACN (15.0 mL) was added TsOH·H₂O (489 mg, 2.57 mmol, 2.40 equiv) and NaNO₂ (177 mg, 2.57 mmol, 2.40 equiv) at 0° C. The mixture was stirred at 0° C. for 0.5 h and CuBr (615 mg, 4.29 mmol, 4.00 equiv) was added at 0° C. The mixture was stirred at 25° C. for 12 h. The mixture was filtered and washed with EtOAc (10.0 mL). The filtrate was diluted with water (10 mL), extracted with ethyl acetate (3×10 mL). The combined organic layer was washed with brine (10 mL), dried over anh Na₂SO₄, filtered and concentrated. The crude residue was purified by reversed phase flash [water (0.1% formic acid)/acetonitrile)] to yield tert-butyl (1R,5R)-6-(3-bromo-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (395 mg, 47.8% yield) as a yellow solid. LCMS [ESI, M+1]: 747.4, 749.4.

To a solution of tert-butyl (1R,5R)-6-(3-bromo-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (450 mg, 602 μmol, 1.00 equiv) in toluene (9.00 mL) and MeOH (9.00 mL) was added Pd₂(dba)₃ (110 mg, 120 μmol, 0.200 equiv), Cs₂CO₃ (490 mg, 1.50 mmol, 2.50 equiv) and t-BuXPhos (102 mg, 241 μmol, 0.400 equiv) under N₂. The mixture was stirred at 70° C. for 3 h. Subsequently, the reaction mixture was concentrated under reduced pressure to remove solvent. The residue was purified by column chromatography (SiO₂, petroleum ether/ethyl acetate, 1:0 to 0:1). The residue was then purified by prep-HPLC [column: Phenomenex Luna C18 150*40 mm*15 μm; mobile phase, A: water (0.1% TFA), B: ACN; B %: 44%-74%; 11 min] to give tert-butyl (1R,5R)-6-(8-fluoro-7-(7-fluoro-8-((triisopropylsilyl)ethynyl)naphthalen-1-yl)-3-methoxy-1,6-naphthyridin-4-yl)-2,6-diazabicyclo[3.2.0]heptane-2-carboxylate (311 mg, 66.5% yield) as a yellow solid. ¹H NMR (400 MHz, CDCl₃): δ 9.36-9.21 (m, 1H), 8.72 (br s, 1H), 8.01-7.85 (m, 2H), 7.66-7.51 (m, 2H), 7.34 (dt, J=2.0, 8.8 Hz, 1H), 5.86-5.53 (m, 1H), 4.96-4.42 (m, 3H), 3.96 (d, J=7.6 Hz, 4H), 3.80-3.63 (m, 1H), 2.54-2.36 (m, 1H), 2.32-2.13 (m, 1H), 1.50 (br s, 9H), 0.94-0.80 (m, 18H), 0.58-0.39 (m, 3H); LCMS [ESI, M+1]: 699.4.

To a solution of tert-butyl (1R,5R)-6-(8-fluoro-7-(7-fluoro-8-((triisopropylsilyl)ethynyl)naphthalen-1-yl)-3-methoxy-1,6-naphthyridin-4-yl)-2,6-diazabicyclo[3.2.0]heptane-2-carboxylate (300 mg, 429 μmol, 1.00 equiv) in DMF (1.50 mL) was added CsF (652 mg, 4.29 mmol, 10.0 equiv). The mixture was stirred at 35° C. for 0.5 h. Subsequently, the reaction mixture was diluted with water (5 mL) and extracted with ethyl acetate (3×5 mL). The combined organic layer was dried over anh Na₂SO₄, filtered and concentrated under reduced pressure. The crude residue was purified by column chromatography (SiO₂, petroleum ether/ethyl acetate, 1:0 to 0:1) to provide tert-butyl (1R,5R)-6-(7-(8-ethynyl-7-fluoronaphthalen-1-yl)-8-fluoro-3-methoxy-1,6-naphthyridin-4-yl)-2,6-diazabicyclo[3.2.0]heptane-2-carboxylate (254 mg, 98.2% yield) as a yellow solid. LCMS [ESI, M+1]: 543.3.

To a solution of tert-butyl (1R,5R)-6-(7-(8-ethynyl-7-fluoronaphthalen-1-yl)-8-fluoro-3-methoxy-1,6-naphthyridin-4-yl)-2,6-diazabicyclo[3.2.0]heptane-2-carboxylate (240 mg, 442 μmol, 1.00 equiv) in ACN (3.00 mL) was added HCl·1,4-dioxane (4.00 M, 3.00 mL, 27.1 equiv). The mixture was stirred at 0° C. for 10 minutes. Subsequently, the reaction mixture was concentrated under reduced pressure to afford 4-((1R,5R)-2,6-diazabicyclo[3.2.0]heptan-6-yl)-7-(8-ethynyl-7-fluoronaphthalen-1-yl)-8-fluoro-3-methoxy-1,6-naphthyridine (211 mg, crude, HCl salt) as a yellow solid. LCMS [ESI, M+1]: 443.0.

To a solution of 4-((1R,5R)-2,6-diazabicyclo[3.2.0]heptan-6-yl)-7-(8-ethynyl-7-fluoronaphthalen-1-yl)-8-fluoro-3-methoxy-1,6-naphthyridine (200 mg, 418 μmol, 1.00 equiv, HCl salt) in DCM (4.00 mL) was added DIEA (270 mg, 2.09 mmol, 364 μL, 5.00 equiv) and prop-2-enoyl prop-2-enoate (79.0 mg, 626 μmol, 1.5 equiv) in DCM (1.00 mL). The mixture was stirred at −40° C. for 10 minutes. Subsequently, the reaction mixture was concentrated under reduced pressure to remove solvent. The crude residue was purified by column chromatography (SiO₂, petroleum ether/ethyl acetate, 1:0 to 0:1). The residue was then purified by prep-HPLC [column: Phenomenex Gemini-NX C18 75*30 mm*3 μm; mobile phase, A: water (10 mM NH₄HCO₃), B: ACN; B %: 26%-56%; 8 min] to afford 1-((1R,5R)-6-(7-(8-ethynyl-7-fluoronaphthalen-1-yl)-8-fluoro-3-methoxy-1,6-naphthyridin-4-yl)-2,6-diazabicyclo[3.2.0]heptan-2-yl)prop-2-en-1-one (95.0 mg, 45.3% over two steps, 98.9% purity) as a white solid. ¹H NMR (400 MHz, CDCl₃): δ 9.24-9.11 (m, 1H), 8.64-8.56 (m, 1H), 8.01-7.90 (m, 2H), 7.69-7.55 (m, 2H), 7.33 (t, J=8.8 Hz, 1H), 6.62-6.29 (m, 2H), 5.86-5.64 (m, 2H), 5.08-4.79 (m, 2H), 4.58 (br d, J=7.6 Hz, 1H), 4.41-4.07 (m, 1H), 4.06-3.81 (m, 4H), 2.82-2.71 (m, 1H), 2.69-2.51 (m, 1H), 2.36-2.15 (m, 1H); LCMS [ESI, M+1]: 497.2.

### Example 7

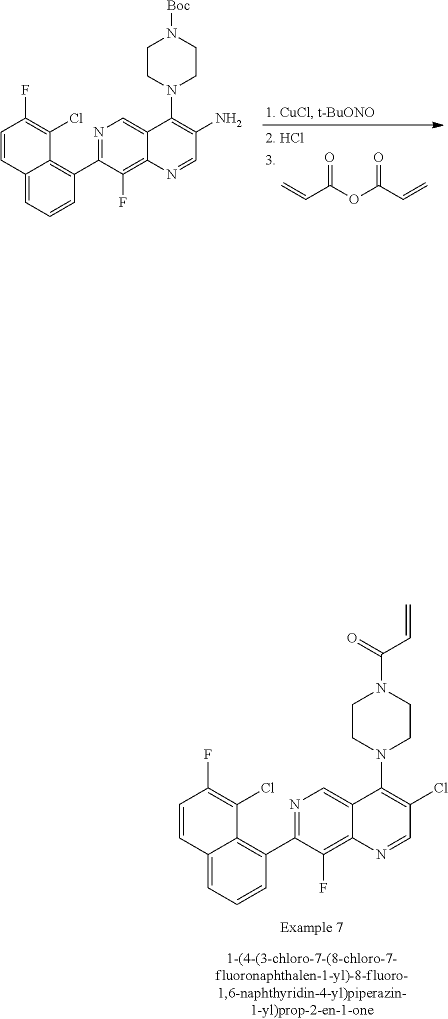

To a suspension of CuCl (33.88 mg, 342 μmol, 1.2 equiv) in MeCN (10 mL) was added tert-butylnitrite (39.7 mg, 385 μmol, 45.8 μL, 1.35 equiv). The mixture was flushed with nitrogen and stirred at 65° C. for 1 h. At this temperature was added tert-butyl 4-(3-amino-7-(8-chloro-7-fluoronaphthalen-1-yl)-8-fluoro-1,6-naphthyridin-4-yl)piperazine-1-carboxylate (150 mg, 285 μmol, 1 equiv) and the mixture was stirred for an additional 3 h. The warm mixture was diluted with EtOAc, washed with brine, dried over anh Na₂SO₄, filtered and concentrated. The residue was purified by prep-TLC (SiO₂, petroleum ether/ethyl acetate, 1:1) to afford tert-butyl 4-(3-chloro-7-(8-chloro-7-fluoronaphthalen-1-yl)-8-fluoro-1,6-naphthyridin-4-yl)piperazine-1-carboxylate (100 mg, 183 μmol, 64% yield) as a yellow solid. LCMS [M+1]: 545.2.

To a solution of tert-butyl 4-(3-chloro-7-(8-chloro-7-fluoronaphthalen-1-yl)-8-fluoro-1,6-naphthyridin-4-yl)piperazine-1-carboxylate (90 mg, 165 μmol, 1 equiv) in EtOAc (3 mL) was added HCl EtOAc (4 M, 12.7 mL, 312 equiv). The mixture was stirred at 25° C. for 10 min. The reaction mixture was concentrated under reduced pressure to provide 3-chloro-7-(8-chloro-7-fluoronaphthalen-1-yl)-8-fluoro-4-(piperazin-1-yl)-1,6-naphthyridine (80 mg, crude) as a yellow solid. LCMS [M+1]: 445.1.

To a solution of 3-chloro-7-(8-chloro-7-fluoronaphthalen-1-yl)-8-fluoro-4-(piperazin-1-yl)-1,6-naphthyridine (70 mg, 157 μmol, 1 equiv) in DCM (5 mL) was added TEA (79.5 mg, 786.0 μmol, 109 μL, 5 equiv) and prop-2-enoyl prop-2-enoate (29.7 mg, 236 μmol, 1.5 equiv) at −40° C. The mixture was stirred at −40° C. for 2 h prior to being diluted with water 10 mL and extracted with DCM (3×20 mL). The combined organic layer was washed with brine (2×10 mL), dried over anh sod sulfate, filtered and concentrated under reduced pressure to give a residue. The residue was purified by prep-HPLC [column: Waters Xbridge Prep OBD C18 150*40 mm*10 um; mobile phase, A: water (10 mM NH₄HCO₃), B: ACN; B %: 35%-65%; 8 min] to give 1-(4-(3-chloro-7-(8-chloro-7-fluoronaphthalen-1-yl)-8-fluoro-1,6-naphthyridin-4-yl)piperazin-1-yl)prop-2-en-1-one (8.35 mg, 16.4 μmol, 10% yield, 98% purity) as a white solid. ¹H NMR (400 MHz, CDCl₃) δ 9.40 (s, 1H), 8.93 (s, 1H), 8.02 (br d, J=7.1 Hz, 1H), 7.91 (dd, J=5.6, 9.0 Hz, 1H), 7.69-7.57 (m, 2H), 7.41 (t, J=8.8 Hz, 1H), 6.65 (dd, J=10.6, 16.8 Hz, 1H), 6.46-6.34 (m, 1H), 5.80 (br d, J=10.4 Hz, 1H), 4.10-3.80 (m, 4H), 3.65 (br s, 4H). LCMS [M+1]: 499.1.

### Example 8

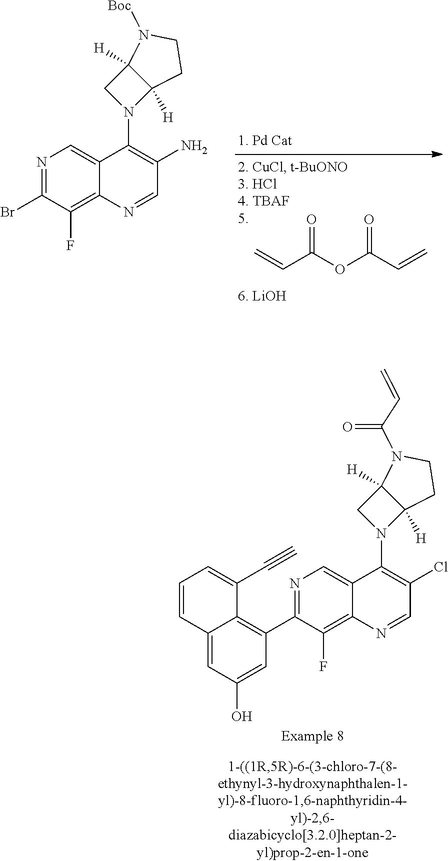

To a mixture of tert-butyl (1R,5R)-6-(3-amino-7-bromo-8-fluoro-1,6-naphthyridin-4-yl)-2,6-diazabicyclo[3.2.0]heptane-2-carboxylate (160 mg, 0.37 mmol, 1.00 equiv) and triisopropyl((6-(methoxymethoxy)-8-(4,4,5,5-tetramethyl-1,3,2-dioxaborolan-2-yl)naphthalen-1-yl)ethynyl)silane (199 mg, 0.40 mmol, 1.10 equiv) in THE (5.00 mL) and H₂O (1.00 mL) was added [2-(2-aminophenyl)phenyl]palladium(1+);dicyclohexyl-[2-(2,4,6-triisopropylphenyl)phenyl]phosphane methanesulfonate (30.9 mg, 36.5 μmol, 0.100 equiv) and K₃PO₄ (155 mg, 0.730 mmol, 2.00 equiv) in one portion at 20° C. under N₂. The mixture was stirred at 60° C. for 16 h prior to being cooled to 20° C. and diluted with water (10.0 mL). The aqueous phase was extracted with EtOAc (2×5.00 mL). The combined organic layer was washed with brine (5.00 mL), dried over anh Na₂SO₄, filtered and concentrated under reduced pressure. The residue was purified by prep-TLC (SiO₂, EtOAc) to give tert-butyl (1R,5R)-6-(3-amino-8-fluoro-7-(3-(methoxymethoxy)-8-((triisopropylsilyl)ethynyl)naphthalen-1-yl)-1,6-naphthyridin-4-yl)-2,6-diazabicyclo[3.2.0]heptane-2-carboxylate (150 mg, 57% yield) as a yellow solid. LCMS [M+1]: 726.5.

To a solution of tert-butyl (1R,5R)-6-(3-amino-8-fluoro-7-(3-(methoxymethoxy)-8-((triisopropylsilyl)ethynyl)naphthalen-1-yl)-1,6-naphthyridin-4-yl)-2,6-diazabicyclo[3.2.0]heptane-2-carboxylate (150 mg, 0.207 mmol, 1.00 equiv) in ACN (2.00 mL) was added CuCl (40.9 mg, 0.413 mmol, 2.00 equiv) and CuCl₂ (83.3 mg, 0.620 mmol, 3.00 equiv) in one portion at 20° C. The mixture was stirred for 10 min. Then the mixture was cooled to 0° C. and a solution of isopentyl nitrite (48.4 mg, 0.413 mmol, 2.00 equiv) in ACN (0.500 mL) was added dropwise at 0° C. The mixture was stirred for 16 h at 20° C. The mixture was diluted with ice-water (10.0 mL) and extracted with EtOAc (2×5.00 mL). The combined organic layer was washed with brine (5.00 mL), dried over anh Na₂SO₄, filtered and concentrated under reduced pressure. The residue was purified by prep-TLC (SiO₂, petroleum ether/ethyl acetate, 1:1) to give tert-butyl (1R,5R)-6-(3-chloro-8-fluoro-7-(3-(methoxymethoxy)-8-((triisopropylsilyl)ethynyl)naphthalen-1-yl)-1,6-naphthyridin-4-yl)-2,6-diazabicyclo[3.2.0]heptane-2-carboxylate (45.0 mg, 29% yield) as a yellow syrup. ¹H NMR (400 MHz, chloroform-d): δ 9.25 (d, J=9.6 Hz, 1H), 8.61 (s, 1H), 7.82 (br d, J=8.0 Hz, 1H), 7.68 (br d, J=6.8 Hz, 1H), 7.53 (s, 1H), 7.45-7.37 (m, 1H), 7.33 (br s, 1H), 6.02-5.64 (m, 1H), 5.36-5.29 (m, 3H), 4.86-4.60 (m, 3H), 3.98 (br d, J=2.8 Hz, 1H), 3.52 (s, 3H), 2.67-2.47 (m, 1H), 2.32-2.19 (m, 1H), 1.50 (br s, 9H), 0.90-0.81 (m, 18H), 0.52-0.44 (m, 3H). LCMS [M+1]: 745.4.

A solution of tert-butyl (1R,5R)-6-(3-chloro-8-fluoro-7-(3-(methoxymethoxy)-8-((triisopropylsilyl)ethynyl)naphthalen-1-yl)-1,6-naphthyridin-4-yl)-2,6-diazabicyclo[3.2.0]heptane-2-carboxylate (70.0 mg, 93.9 μmol, 1.00 equiv) in HCl-EtOAc (3.00 mL, 4 N) was stirred for 30 min at 20° C. The mixture was concentrated under reduced pressure to provide 4-(4-((1R,5R)-2,6-diazabicyclo[3.2.0]heptan-6-yl)-3-chloro-8-fluoro-1,6-naphthyridin-7-yl)-5-((triisopropylsilyl)ethynyl)naphthalen-2-ol hydrochloride (59.0 mg, 92.5 μmol, 99% yield) as light-yellow solid. LCMS [M+1]: 601.

To a solution of 4-(4-((1R,5R)-2,6-diazabicyclo[3.2.0]heptan-6-yl)-3-chloro-8-fluoro-1,6-naphthyridin-7-yl)-5-((triisopropylsilyl)ethynyl)naphthalen-2-ol hydrochloride (59.0 mg, 92.5 μmol, 1.00 eq) in THE (2.00 mL) was added TEA (28.1 mg, 278 μmol, 3.00 equiv) and TBAF (185 uL, 185 μmol, 1 M in THF, 2.00 equiv) dropwise at 20° C. The mixture was stirred for 1 h prior to being concentrated under reduced pressure to give 4-(4-((1R,5R)-2,6-diazabicyclo[3.2.0]heptan-6-yl)-3-chloro-8-fluoro-1,6-naphthyridin-7-yl)-5-ethynylnaphthalen-2-ol (40.0 mg, crude) as a yellow syrup.

To a solution of 4-(4-((1R,5R)-2,6-diazabicyclo[3.2.0]heptan-6-yl)-3-chloro-8-fluoro-1,6-naphthyridin-7-yl)-5-ethynylnaphthalen-2-ol (40.0 mg, 89.9 μmol, 1.00 equiv) and TEA (27.3 mg, 270 μmol, 3.00 equiv) in DCM (2.00 mL) was added a solution of prop-2-enoyl prop-2-enoate (22.7 mg, 180 μmol, 2.00 equiv) in DCM (0.200 mL) dropwise at −40° C. under N₂. The mixture was stirred for 10 min. The mixture was diluted with water (5.00 mL) and extracted with DCM (2×3 mL). The combined organic layer was washed with brine (3.00 mL), dried over anh Na₂SO₄, filtered and concentrated under reduced pressure to afford 4-(4-((1R,5R)-2-acryloyl-2,6-diazabicyclo[3.2.0]heptan-6-yl)-3-chloro-8-fluoro-1,6-naphthyridin-7-yl)-5-ethynylnaphthalen-2-yl acrylate (50.0 mg, crude) was obtained as yellow syrup.

To a solution of 4-(4-((1R,5R)-2-acryloyl-2,6-diazabicyclo[3.2.0]heptan-6-yl)-3-chloro-8-fluoro-1,6-naphthyridin-7-yl)-5-ethynylnaphthalen-2-yl acrylate (50.0 mg, 90.4 μmol, 1.00 equiv) in THE (2.00 mL) and H₂O (0.500 mL) was added LiOH—H₂O (11.4 mg, 271 μmol, 3.00 equiv) in one portion at 20° C. The mixture was stirred for 16 h. The mixture was diluted with water (5.00 mL) and extracted with DCM (2×3.00 mL). The combined organic layer was washed with brine (3.00 mL), dried over anh Na₂SO₄, filtered and concentrated under reduced pressure. The residue was purified by prep-TLC (EtOAc/EtOH, 10:1) to give 1-((1R,5R)-6-(3-chloro-7-(8-ethynyl-3-hydroxynaphthalen-1-yl)-8-fluoro-1,6-naphthyridin-4-yl)-2,6-diazabicyclo[3.2.0]heptan-2-yl)prop-2-en-1-one (12.7 mg, 28% over three steps) as a light-yellow solid. ¹H NMR (400 MHz, chloroform-d): δ 9.08 (br s, 1H), 8.55 (s, 1H), 7.66 (br d, J=7.6 Hz, 1H), 7.54-7.43 (m, 1H), 7.33-7.28 (m, 1H), 7.24-7.12 (m, 2H), 6.60-6.16 (m, 2H), 5.95-5.60 (m, 2H), 5.07-4.62 (m, 3H), 4.43-3.59 (m, 2H), 2.67-2.46 (m, 1H), 2.39 (s, 1H), 2.36-2.12 (m, 1H). LCMS [M+1]: 499.

### Example 9

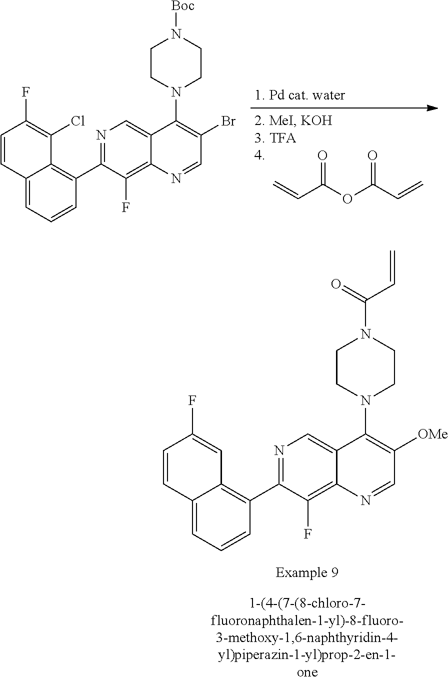

To a solution of tert-butyl 4-(3-bromo-7-(8-chloro-7-fluoronaphthalen-1-yl)-8-fluoro-1,6-naphthyridin-4-yl)piperazine-1-carboxylate (200 mg, 340 μmol, 1 equiv) in dioxane (4.00 mL) and H₂O (1.20 mL) was added t-Bu Xphos (28.8 mg, 67.8 μmol, 0.2 equiv), Cs₂CO₃ (440 mg, 678 μmol, 4 equiv) and Pd₂(dba)₃ (31.0 mg, 33.8 μmol, 0.1 equiv). The mixture was stirred at 90° C. for 2 h prior to being diluted with H₂O (50 mL) and extracted with ethyl acetate (3×10 mL). The combined organic layer was washed with brine (10 mL), dried over anh Na₂SO₄, filtered and concentrated under reduced pressure to give a residue. The residue was purified by prep-TLC (ethyl acetate) to afford tert-butyl 4-(7-(8-chloro-7-fluoronaphthalen-1-yl)-8-fluoro-3-hydroxy-1,6-naphthyridin-4-yl)piperazine-1-carboxylate (100 mg, 190 μmol, 56% yield) as a white solid. LCMS [ESI, M+1]: 527.4.

To a solution of tert-butyl 4-(7-(8-chloro-7-fluoronaphthalen-1-yl)-8-fluoro-3-hydroxy-1,6-naphthyridin-4-yl)piperazine-1-carboxylate (100 mg, 189 μmol, 1 equiv) in DMSO (1.00 mL) was added KOH (31.9 mg, 569 μmol, 1 equiv) and MeI (26.9 mg, 189 μmol, 1 equiv). The mixture was stirred at 20° C. for 1 h prior to being diluted with H₂O (10 mL) and extracted with EA (3×10 mL). The combined organic layer was washed with brine (10 mL), dried over anh Na₂SO₄, filtered and concentrated under reduced pressure to give a residue. The residue was purified by prep-TLC (petroleum ether/ethyl acetate, 1:1) followed by prep-HPLC [column: Waters Xbridge Prep OBD C18 100×25 mm×5 um; mobile phase, A: water (10 mM NH₄HCO₃), B: ACN; B %: 55%-75%; 10 min) to provide tert-butyl 4-(7-(8-chloro-7-fluoronaphthalen-1-yl)-8-fluoro-3-methoxy-1,6-naphthyridin-4-yl)piperazine-1-carboxylate (30 mg, 52.7 μmol, 28% yield) as a white solid. LCMS [ESI, M+1]: 541.3.

To a solution of tert-butyl 4-(7-(8-chloro-7-fluoronaphthalen-1-yl)-8-fluoro-3-methoxy-1,6-naphthyridin-4-yl)piperazine-1-carboxylate (30.0 mg, 55.4 μmol, 1 equiv) in DCM (3.00 mL) was added TFA (924 mg, 8.10 mmol, 0.60 mL). The mixture was stirred at 0° C. for 1 hour. Subsequently, the reaction mixture was concentrated under reduced pressure to provide 7-(8-chloro-7-fluoronaphthalen-1-yl)-8-fluoro-3-methoxy-4-(piperazin-1-yl)-1,6-naphthyridine (30.0 mg, crude) as a yellow solid.

To a solution of 7-(8-chloro-7-fluoronaphthalen-1-yl)-8-fluoro-3-methoxy-4-(piperazin-1-yl)-1,6-naphthyridine (30.0 mg, 1 equiv) in DCM (1.00 mL) was added TEA (20.6 mg, 204 μmol, 28.4 μL, 3 equiv) and prop-2-enoyl prop-2-enoate (12.8 mg, 102 μmol, 1.5 equiv) at −40° C. The mixture was stirred at −40° C. for 1 hour prior to being diluted with water (10 mL) and then extracted with DCM (3×10 mL). The combined organic layer was washed with brine (3×10 mL), dried over anh Na₂SO₄, filtered and concentrated under reduced pressure to give a residue. The residue was purified by prep-TLC to afford 1-(4-(7-(8-chloro-7-fluoronaphthalen-1-yl)-8-fluoro-3-methoxy-1,6-naphthyridin-4-yl)piperazin-1-yl)prop-2-en-1-one (16.53 mg, 32.9 μmol, 48% over two steps) as a white solid. ¹H NMR (400 MHz, CDCl₃): δ 9.42 (s, 1H), 8.96 (s, 1H), 8.01 (dd, J=1.2, 8.0 Hz, 1H), 7.90 (dd, J=5.6, 9.2 Hz, 1H), 7.67-7.59 (m, 2H), 7.40 (t, J=8.8 Hz, 1H), 6.66 (dd, J=10.4, 16.4 Hz, 1H), 6.38 (dd, J=1.6, 16.8 Hz, 1H), 5.78 (dd, J=2.0, 10.8 Hz, 1H), 4.11 (s, 3H), 3.95-3.77 (m, 4H), 3.49 (t, J=4.8 Hz, 4H). LCMS [ESI, M+1]: 495.1.

### Example 10

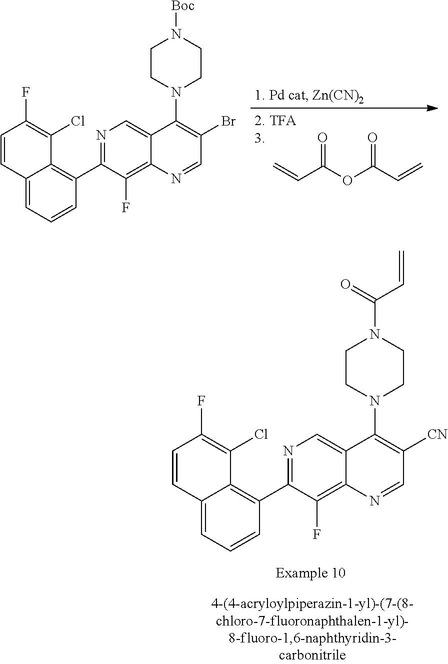

To a solution of tert-butyl 4-(3-bromo-7-(8-chloro-7-fluoronaphthalen-1-yl)-8-fluoro-1,6-naphthyridin-4-yl)piperazine-1-carboxylate (120 mg, 203 μmol, 1 equiv) in DMF (1.00 mL) was added Zn(CN)₂ (23.9 mg, 203 μmol, 1 equiv) and Pd(PPh₃)₄ (23.5 mg, 20.3 μmol, 0.1 equiv). The mixture was stirred at 100° C. for 2 h. The reaction was diluted with saturated NaHCO₃ (50 mL) and extracted with EtOAc (3×10 mL). The combined organic layer was washed with brine (10 mL), dried over anh Na₂SO₄, filtered and concentrated under reduced pressure to give a residue. The residue was purified by prep-TLC (petroleum ether/ethyl acetate, 1:1) to provide tert-butyl 4-(7-(8-chloro-7-fluoronaphthalen-1-yl)-3-cyano-8-fluoro-1,6-naphthyridin-4-yl)piperazine-1-carboxylate (60 mg, 111 μmol, 55% yield) as a white solid. LCMS [ESI, M+1]: 536.2.

To a solution of tert-butyl 4-(7-(8-chloro-7-fluoronaphthalen-1-yl)-3-cyano-8-fluoro-1,6-naphthyridin-4-yl)piperazine-1-carboxylate (60.0 mg, 111 μmol, 1 equiv) in DCM (1.00 mL) was added TFA (308 mg, 2.70 mmol, 0.2 mL, 24 equiv). The mixture was stirred at 0° C. for 1 hour. The reaction mixture was concentrated under reduced pressure to provide 7-(8-chloro-7-fluoronaphthalen-1-yl)-8-fluoro-4-(piperazin-1-yl)-1,6-naphthyridine-3-carbonitrile (40.0 mg, crude) as a yellow solid. LCMS [ESI, M+1]: 436.1.

To a solution of 7-(8-chloro-7-fluoronaphthalen-1-yl)-8-fluoro-4-(piperazin-1-yl)-1,6-naphthyridine-3-carbonitrile (40.0 mg, 91.7 μmol, 1 equiv) in DCM (5.00 mL) was added TEA (27.8 mg, 275 μmol, 38.3 μL, 3 equiv) and prop-2-enoyl prop-2-enoate (17.3 mg, 137 μmol, 1.5 equiv) at −40° C. The mixture was stirred at −40° C. for 1 hour. The reaction mixture was diluted with water (10 mL) and extracted with DCM (3×10 mL). The combined organic layer was washed with brine (3×10 mL), dried over anh Na₂SO₄, filtered and concentrated under reduced pressure to give a residue. The residue was purified by prep-HPLC [column: Waters Xbridge Prep OBD C18 150×40 mm×10 um; mobile phase, A: water (10 mM NH₄HCO₃), B: ACN; B %: 35%-65%; 8 min) to yield 4-(4-acryloylpiperazin-1-yl)-7-(8-chloro-7-fluoronaphthalen-1-yl)-8-fluoro-1,6-naphthyridine-3-carbonitrile (7.36 mg, 14.5 μmol, 16% over two steps) as a white solid. ¹H NMR (400 MHz, CDCl₃): δ 9.28 (s, 1H), 8.97 (s, 1H), 8.05 (dd, J=2.4, 7.6 Hz, 1H), 7.96-7.90 (m, 1H), 7.69-7.61 (m, 2H), 7.47-7.40 (m, 1H), 6.70-6.61 (m, 1H), 6.42 (dd, J=1.6, 16.8 Hz, 1H), 5.84 (dd, J=1.6, 10.4 Hz, 1H), 4.09-3.91 (m, 8H). LCMS [ESI, M+1]: 490.1.

### Example 11

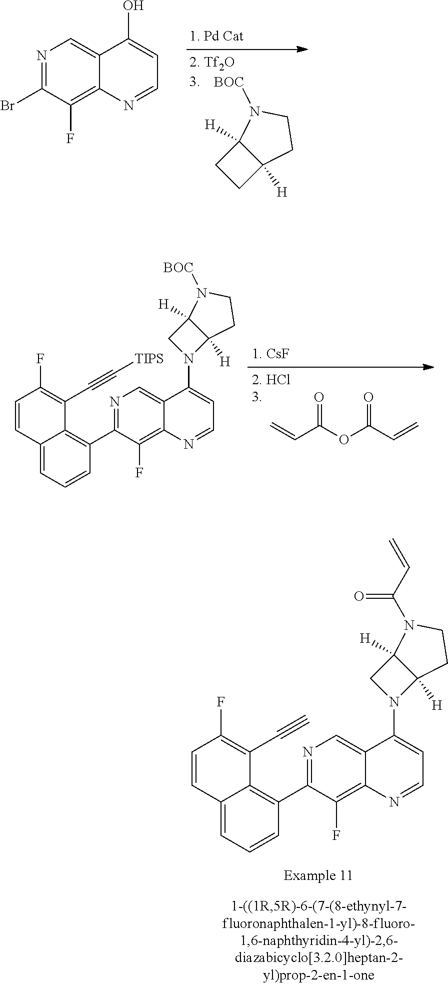

To a solution of 7-bromo-8-fluoro-1,6-naphthyridin-4-ol (1.00 g, 4.11 mmol, 1.0 equiv) in EtOH (35 mL) was added Ad₂n-BuP-Pd-G3 (300 mg, 411 μmol, 0.1 equiv), K₃PO₄ (1.5 M, 11.0 mL, 4.0 equiv), and ((2-fluoro-8-(4,4,5,5-tetramethyl-1,3,2-dioxaborolan-2-yl)naphthalen-1-yl)ethynyl)triisopropylsilane (3.72 g, 8.23 mmol, 2.0 equiv). The mixture was stirred at 80° C. for 6 hours. Subsequently, the reaction mixture was concentrated under reduced pressure, was diluted with water (20 mL), and extracted with ethyl acetate (3×30 mL). The combined organic layer was washed with brine (30 mL), dried over anh Na₂SO₄, filtered and concentrated under reduced pressure. The residue was purified by reversed phase flash [water (0.1% formic acid)/acetonitrile)] affording 8-fluoro-7-(7-fluoro-8-((triisopropylsilyl)ethynyl)naphthalen-1-yl)-1,6-naphthyridin-4-ol (545 mg, 22% yield) as a yellow solid. LCMS [ESI, M+1]: 489.1.

To a solution of 8-fluoro-7-(7-fluoro-8-((triisopropylsilyl)ethynyl)naphthalen-1-yl)-1,6-naphthyridin-4-ol (530 mg, 1.08 mmol, 1.0 equiv) in DCM (10 mL) was added 4 Å MS (360 mg) at 20° C. followed by DIEA (701 mg, 5.42 mmol, 945 μL, 5.0 equiv), and finally Tf₂O (1.01 g, 3.58 mmol, 590 μL, 3.3 equiv) over a period of 10 minutes. The mixture was stirred at −40° C. for 1 hour. After 1 hour, Tf₂O (459 mg, 1.63 mmol, 268 μL, 1.5 equiv) was added and the mixture was stirred at −40° C. for 0.5 hours. Subsequently, the reaction mixture was diluted with water (10 mL) and extracted with ethyl acetate (3×10 mL). The combined organic layer was washed with brine (10 mL), dried over anh Na₂SO₄, filtered and concentrated under reduced pressure. The residue was purified by column chromatography (SiO₂, petroleum ether/ethyl acetate, 100:1 to 10:1) to provide 8-fluoro-7-(7-fluoro-8-((triisopropylsilyl)ethynyl)naphthalen-1-yl)-1,6-naphthyridin-4-yl trifluoromethanesulfonate (290 mg, 41% yield) as a yellow solid. LCMS [ESI, M+1]: 621.3.

To a solution of 8-fluoro-7-(7-fluoro-8-((triisopropylsilyl)ethynyl)naphthalen-1-yl)-1,6-naphthyridin-4-yl trifluoromethanesulfonate (290 mg, 467 μmol, 1.0 equiv) in toluene (20.0 mL) was added tert-butyl (1R,5R)-2,6-diazabicyclo[3.2.0]heptane-2-carboxylate (139 mg, 701 μmol, 1.5 equiv), Pd₂(dba)₃ (42.8 mg, 46.7 μmol, 0.1 equiv), RuPhos (43.6 mg, 93.4 μmol, 0.2 equiv), 4 Å MS (290 mg) and Cs₂CO₃ (380 mg, 1.17 mmol, 2.5 equiv). The mixture was stirred at 100° C. for 12 hours. The reaction mixture was diluted with water (20 mL) and extracted with ethyl acetate (3×20 mL). The combined organic layer was dried over anh Na₂SO₄, filtered and concentrated under reduced pressure. The resultant residue was purified by reversed phase flash [water (0.1% formic acid)/acetonitrile)] to afford tert-butyl (1R,5R)-6-(8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (220 mg, 65% yield) as a yellow solid. LCMS [ESI, M+1]: 669.6.

To a solution of tert-butyl (1R,5R)-6-(8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (210 mg, 314 μmol, 1.0 equiv) in DMF (1.0 mL) was added CsF (477 mg, 3.14 mmol, 10 equiv). The mixture was stirred at 35° C. for 30 minutes. Subsequently, the reaction mixture was diluted with water (2 mL) and extracted with ethyl acetate (3×3 mL). The combined organic layer was washed with brine (3 mL), dried over anh Na₂SO₄, filtered and concentrated under reduced pressure to provide the crude residue. The residue was purified by reversed phase flash [water (0.1% formic acid)/acetonitrile)] to give tert-butyl (1R,5R)-6-(7-(8-ethynyl-7-fluoronaphthalen-1-yl)-8-fluoro-1,6-naphthyridin-4-yl)-2,6-diazabicyclo[3.2.0]heptane-2-carboxylate (162 mg, 99% yield) as a yellow solid. LCMS [ESI, M+1]: 513.2.

To a solution of tert-butyl (1R,5R)-6-(7-(8-ethynyl-7-fluoronaphthalen-1-yl)-8-fluoro-1,6-naphthyridin-4-yl)-2,6-diazabicyclo[3.2.0]heptane-2-carboxylate (130 mg, 256 μmol, 1.0 equiv) in ACN (1.0 mL) was added HCl-dioxane (4 M, 1 mL, 15.8 equiv). The mixture was stirred at 0° C. for 10 minutes. The mixture was concentrated, then the reaction mixture was diluted with water (4 mL) and adjusted to pH˜8 with saturated aq NaHCO₃. The aqueous layer was extracted with DCM (3×2 mL) and the combined organic layer was washed with brine (2 mL), dried over anh Na₂SO₄, filtered and concentrated to give 4-((1R,5R)-2,6-diazabicyclo[3.2.0]heptan-6-yl)-7-(8-ethynyl-7-fluoronaphthalen-1-yl)-8-fluoro-1,6-naphthyridine (140 mg, crude) as a white solid. LCMS [ESI, M+1]: 413.0.

To a solution of 4-((1R,5R)-2,6-diazabicyclo[3.2.0]heptan-6-yl)-7-(8-ethynyl-7-fluoronaphthalen-1-yl)-8-fluoro-1,6-naphthyridine (130 mg, 315 μmol, 1.0 equiv) in DCM (0.5 mL) was added DIEA (204 mg, 1.58 mmol, 274 μL, 5.0 equiv) and prop-2-enoyl prop-2-enoate (59.6 mg, 473 μmol, 1.5 equiv) in DCM (0.5 mL). The mixture was stirred at −40° C. for 10 minutes. The reaction mixture was diluted with satd aq NH₄Cl and extracted with ethyl acetate (3×5 mL). The combined organic layer was washed with brine (5 mL), dried over anh Na₂SO₄, filtered and concentrated to provide a crude residue. The residue was purified by prep-HPLC [column: Waters Xbridge 150*25 mm*5 μm; mobile phase, A: water (10 mM NH₄HCO₃), B: ACN; B %: 26%-56%; 10 min]. The desired fractions were collected and concentrated under vacuum to remove acetonitrile. The mixture was lyophilized to afford 1-((1R,5R)-6-(7-(8-ethynyl-7-fluoronaphthalen-1-yl)-8-fluoro-1,6-naphthyridin-4-yl)-2,6-diazabicyclo[3.2.0]heptan-2-yl)prop-2-en-1-one (42 mg, 28% yield). ¹H NMR (400 MHz, CDCl₃): δ 9.21-9.09 (m, 1H), 8.80-8.67 (m, 1H), 8.05-7.92 (m, 2H), 7.72-7.51 (m, 2H), 7.34 (t, J=8.8 Hz, 1H), 6.64-6.31 (m, 3H), 5.82 (br dd, J=2.0, 9.6 Hz, 1H), 5.52-5.29 (m, 1H), 5.25-4.90 (m, 1H), 4.73 (td, J=6.4, 9.2 Hz, 1H), 4.49-4.16 (m, 2H), 4.06-3.77 (m, 1H), 2.71 (d, J=10.4 Hz, 1H), 2.62-2.42 (m, 1H), 2.36-2.10 (m, 1H). LCMS [ESI, M+1]: 467.2.

### Example 12

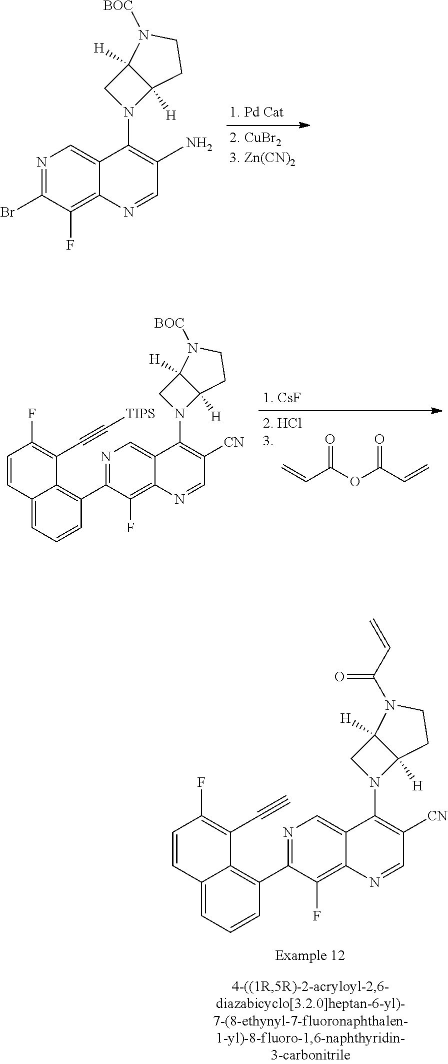

To a solution of tert-butyl (1R,5R)-6-(3-amino-7-bromo-8-fluoro-1,6-naphthyridin-4-yl)-2,6-diazabicyclo[3.2.0]heptane-2-carboxylate (1 g, 2.28 mmol, 1.0 equiv) and ((2-fluoro-8-(4,4,5,5-tetramethyl-1,3,2-dioxaborolan-2-yl)naphthalen-1-yl)ethynyl)triisopropylsilane (1.14 g, 2.51 mmol, 1.1 equiv) in toluene (20 mL) was added K₃PO₄ (1.5 M in water, 4.56 mL, 3.0 equiv) and [2-(2-aminophenyl)phenyl]palladium(1+);bis(1-adamantyl)-butyl-phosphane methanesulfonate (166 mg, 228 μmol, 0.1 equiv). After stirring at 90° C. for 1.5 hours under N₂, the mixture was diluted with water (10 mL) and extracted with ethyl acetate (2×15 mL). The combined organic layer was dried over anh Na₂SO₄, filtered and concentrated to provide a crude residue. The residue was purified by reversed phase flash chromatography [water (0.1% FA)/acetonitrile]. The desired fractions were collected and neutralized with solid NaHCO₃, concentrated under vacuum to remove MeCN and extracted with ethyl acetate (2×20 mL). The combined organic layer was dried over anh Na₂SO₄, filtered and concentrated under vacuum to afford tert-butyl (1R,5R)-6-(3-amino-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (1.22 g, 74%) as a yellow solid. ¹H NMR (400 MHz, CDCl₃): δ 9.27 (s, 1H), 8.52-8.44 (m, 1H), 7.96-7.87 (m, 2H), 7.63-7.58 (m, 1H), 7.58-7.52 (m, 1H), 7.33 (dt, J=2.4, 8.8 Hz, 1H), 5.80-5.49 (m, 1H), 4.74-4.33 (m, 3H), 4.04-3.93 (m, 1H), 3.88-3.69 (m, 1H), 3.55-3.44 (m, 2H), 2.42-2.31 (m, 1H), 2.29-2.15 (m, 1H), 1.53-1.49 (m, 9H), 0.90-0.81 (m, 18H), 0.57-0.35 (m, 3H). LCMS [M+1]: 684.2.

To a solution of tert-butyl (1R,5R)-6-(3-amino-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (1.22 g, 1.78 mmol, 1.0 eq) in MeCN (20 mL) was added CuBr (512 mg, 3.57 mmol, 2.0 equiv) and CuBr₂ (1.20 g, 5.35 mmol, 3.0 equiv) in one portion at 20° C. The mixture was stirred for 10 minutes. The mixture was cooled to 0° C. and a solution of isopentyl nitrite (418 mg, 3.57 mmol, 480 μL, 2.0 equiv) in MeCN (1 mL) was added dropwise at 0° C. After stirring at 20° C. for 16 hours the mixture was diluted with water (30 mL) and extracted with ethyl acetate (2×40 mL). The combined organic layer was dried over anh Na₂SO₄ and concentrated under vacuum. The residue was purified by silica gel chromatography (petroleum ether/ethyl acetate, 3:1 to 1:1) to give tert-butyl (1R,5R)-6-(3-bromo-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (0.6 g, 40%) as a yellow solid. ¹H NMR (400 MHz, CDCl₃): δ 9.24 (d, J=9.6 Hz, 1H), 8.77-8.71 (m, 1H), 7.98-7.88 (m, 2H), 7.63-7.53 (m, 2H), 7.38-7.31 (m, 1H), 6.09-5.78 (m, 1H), 4.81-4.56 (m, 3H), 4.06-3.93 (m, 1H), 3.82-3.62 (m, 1H), 2.64-2.46 (m, 1H), 2.37-2.22 (m, 1H), 1.55-1.42 (m, 9H), 0.91-0.84 (m, 18H), 0.55-0.39 (m, 3H). LCMS [M+1]: 747.0, 749.0.

To a solution of tert-butyl (1R,5R)-6-(3-bromo-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (150 mg, 200 μmol, 1 equiv) in DMF (1.5 mL) was added Zn(CN)₂ (110 mg, 937 μmol, 4.67 equiv) and Pd(PPh₃)₄ (23.2 mg, 20.1 μmol, 0.1 equiv). The mixture was stirred at 100° C. for 1.5 hours under N₂. Subsequently, the mixture was diluted with satd aq NaHCO₃ solution (5 mL) and extracted with EtOAc (3×5 mL). The combined organic layer was washed with brine (10 mL), dried over anh Na₂SO₄, filtered and concentrated to provide a residue. The residue was purified by reversed phase flash [water (0.1% FA)/acetonitrile to methanol]. The desired fractions were collected and concentrated under vacuum to give tert-butyl (1R,5R)-6-(3-cyano-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (135 mg, 87%) as a brown solid. ¹H NMR (400 MHz, CDCl₃): δ 9.27-9.20 (m, 1H), 8.73 (d, J=2.0 Hz, 1H), 8.01-7.90 (m, 2H), 7.62-7.51 (m, 2H), 7.40-7.32 (m, 1H), 5.97-5.84 (m, 1H), 5.04-4.62 (m, 3H), 4.10-3.98 (m, 1H), 3.78-3.61 (m, 1H), 2.67-2.51 (m, 1H), 2.46-2.30 (m, 1H), 1.51 (s, 9H), 0.91-0.84 (m, 18H), 0.60-0.46 (m, 3H). LCMS [M+1]: 694.1.

To a solution of tert-butyl (1R,5R)-6-(3-cyano-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (180 mg, 259 μmol, 1.0 equiv) in DMF (2 mL) was added CsF (394 mg, 2.59 mmol, 10 equiv). After stirring at 25° C. for 1 hour, the mixture was filtered and the filtrate was purified by reversed phase flash chromatography [water (0.1% FA)/acetonitrile, 0-100%]. The desired fractions were collected and concentrated under vacuum to give tert-butyl (1R,5R)-6-(3-cyano-7-(8-ethynyl-7-fluoronaphthalen-1-yl)-8-fluoro-1,6-naphthyridin-4-yl)-2,6-diazabicyclo[3.2.0]heptane-2-carboxylate (130 mg, 90%) as a yellow solid. LCMS [M+1]: 538.3.

To a solution of tert-butyl (1R,5R)-6-(3-cyano-7-(8-ethynyl-7-fluoronaphthalen-1-yl)-8-fluoro-1,6-naphthyridin-4-yl)-2,6-diazabicyclo[3.2.0]heptane-2-carboxylate (130 mg, 241.8 μmol, 1 equiv) in MeCN (0.3 mL) was added HCl·dioxane (4 M, 605 μL, 10 equiv). After stirring at 25° C. for 0.5 hour, the mixture was concentrated under vacuum to give 4-((1R,5R)-2,6-diazabicyclo[3.2.0]heptan-6-yl)-7-(8-ethynyl-7-fluoronaphthalen-1-yl)-8-fluoro-1,6-naphthyridine-3-carbonitrile (0.12 g, crude, HCl salt) as a yellow solid. R_{f}=0.10 (ethyl acetate/methanol, 10:1). LCMS [ESI, M+1]: 438.2.

To a solution of 4-((1R,5R)-2,6-diazabicyclo[3.2.0]heptan-6-yl)-7-(8-ethynyl-7-fluoronaphthalen-1-yl)-8-fluoro-1,6-naphthyridine-3-carbonitrile (115 mg, 243 μmol, 1.0 equiv, HCl salt) in dichloromethane (3 mL) was added TEA (98.2 mg, 971 μmol, 135 μL, 4.0 equiv) and prop-2-enoyl prop-2-enoate (36.7 mg, 291 μmol, 1.2 equiv) at −40° C. The mixture was stirred at −40° C. for 0.25 hour. Subsequently, the mixture was diluted with water (3 mL) and layers were separated. The aqueous phase was extracted with ethyl acetate (2×3 mL). The combined organic layer was dried over Na₂SO₄, filtered and concentrated under vacuum. The residue was purified by chromatography (Al₂O₃, ethyl acetate/methanol, 1:0 to 30:1) then by prep-HPLC [column: Phenomenex Gemini-NX C18 75*30 mm*3 um; mobile phase, A: water (0.05% ammonia hydroxide v/v), B: ACN; B %: 27%-57%; 7 min] to afford 4-((1R,5R)-2-acryloyl-2,6-diazabicyclo[3.2.0]heptan-6-yl)-7-(8-ethynyl-7-fluoronaphthalen-1-yl)-8-fluoro-1,6-naphthyridine-3-carbonitrile (14 mg, 12% over two steps) as an off-white solid. ¹H NMR (400 MHz, CDCl₃): δ 9.22-9.13 (m, 1H), 8.80-8.71 (m, 1H), 8.05-7.93 (m, 2H), 7.67-7.57 (m, 2H), 7.42-7.32 (m, 1H), 6.63-6.29 (m, 2H), 6.11-5.89 (m, 1H), 5.89-5.78 (m, 1H), 5.30-4.94 (m, 2H), 4.87-4.69 (m, 1H), 4.53-4.12 (m, 1H), 4.08-3.68 (m, 1H), 2.88-2.64 (m, 2H), 2.61-2.30 (m, 1H). LCMS [ESI, M+1]: 492.2.

### Example 13

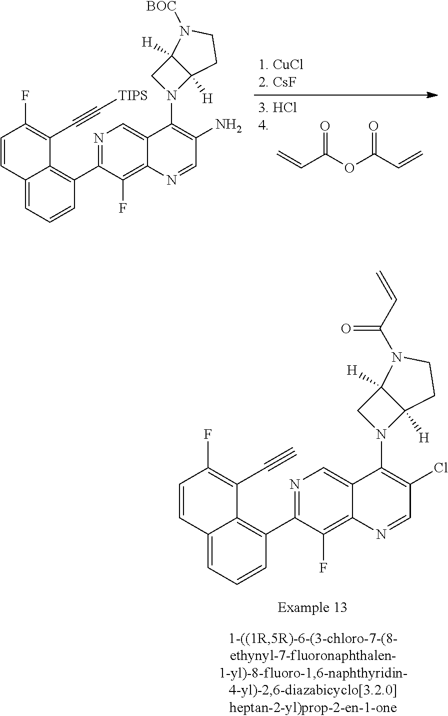

To a solution of tert-butyl (1R,5R)-6-(3-amino-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (1.05 g, 1.54 mmol, 1 equiv) in MeCN (20 mL) was added CuCl₂ (619 mg, 4.61 mmol, 3 equiv) and CuCl (304 mg, 3.07 mmol, 2 equiv) in one portion at 20° C. The mixture was stirred for 10 minutes and was cooled to 0° C. To this mixture was added dropwise a solution of isopentyl nitrite (360 mg, 3.07 mmol, 413 μL, 2 equiv) in MeCN (2 mL). The mixture was stirred for 16 hours at 20° C. Subsequently, the mixture was diluted with water (30 mL), filtered and the filtrate was concentrated. The aqueous phase was and extracted with ethyl acetate (2×40 mL). The combined organic layer was dried over anh Na₂SO₄, filtered and concentrated to provide a crude residue. The residue was purified by silica gel chromatography (petroleum ether/ethyl acetate, 3:1 to 1:1) to afford tert-butyl (1R,5R)-6-(3-chloro-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (0.6 g, 50%) as a yellow oil. LCMS [ESI, M+1]: 703.3.

To a solution of tert-butyl (1R,5R)-6-(3-chloro-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (0.6 g, 853 μmol, 1 equiv) in DMF (6 mL) was added CsF (1.30 g, 8.53 mmol, 10 equiv). The mixture was stirred at 25° C. for 1 hour. Subsequently, the mixture was filtered and the filtrate was purified by reversed phase flash chromatography [water (0.1% FA)/acetonitrile, 0-100%] to give tert-butyl (1R,5R)-6-(3-chloro-7-(8-ethynyl-7-fluoronaphthalen-1-yl)-8-fluoro-1,6-naphthyridin-4-yl)-2,6-diazabicyclo[3.2.0]heptane-2-carboxylate (0.4 g, 77%) as a yellow oil.

To a solution of tert-butyl (1R,5R)-6-(3-chloro-7-(8-ethynyl-7-fluoronaphthalen-1-yl)-8-fluoro-1,6-naphthyridin-4-yl)-2,6-diazabicyclo[3.2.0]heptane-2-carboxylate (400 mg, 731 μmol, 1.0 equiv) in ACN (1.3 mL) was added HCl·dioxane (4 M, 2.74 mL, 15 equiv). The mixture was stirred at 15° C. for 20 minutes. The reaction mixture was concentrated under vacuum to give 4-((1R,5R)-2,6-diazabicyclo[3.2.0]heptan-6-yl)-3-chloro-7-(8-ethynyl-7-fluoronaphthalen-1-yl)-8-fluoro-1,6-naphthyridine (355 mg, 95%, HCl salt) as a yellow solid. LCMS [ESI, M+1]: 446.9.

To a solution of 4-((1R,5R)-2,6-diazabicyclo[3.2.0]heptan-6-yl)-3-chloro-7-(8-ethynyl-7-fluoronaphthalen-1-yl)-8-fluoro-1,6-naphthyridine (353 mg, 730 μmol, 1.0 equiv, HCl salt) in DCM (7 mL) was added TEA (296 mg, 2.92 mmol, 407 μL, 4.0 equiv) and prop-2-enoyl prop-2-enoate (110 mg, 876 μmol, 1.2 equiv) at −40° C. The mixture was stirred at −40° C. for 0.25 hour. The mixture was diluted with methanol (0.1 mL) and water (8 mL). The aqueous phase was extracted with ethyl acetate (8 mL). The combined organic layer was dried over anh Na₂SO₄, filtered and concentrated to provide a crude residue. The residue was purified by reversed phase flash chromatography [water/acetonitrile, 0-70%] to afford 1-((1R,5R)-6-(3-chloro-7-(8-ethynyl-7-fluoronaphthalen-1-yl)-8-fluoro-1,6-naphthyridin-4-yl)-2,6-diazabicyclo[3.2.0]heptan-2-yl)prop-2-en-1-one (100 mg, 26%) as a yellow solid. ¹H NMR (400 MHz, CDCl₃): δ 9.23-9.15 (m, 1H), 8.65-8.59 (m, 1H), 8.03-7.91 (m, 2H), 7.67-7.56 (m, 2H), 7.39-7.30 (m, 1H), 6.62-6.28 (m, 2H), 6.12-5.91 (m, 1H), 5.86-5.76 (m, 1H), 5.14-4.85 (m, 2H), 4.83-4.71 (m, 1H), 4.46-4.08 (m, 1H), 4.07-3.79 (m, 1H), 2.88-2.58 (m, 2H), 2.49-2.25 (m, 1H). LCMS [ESI, M+1]: 501.2.

### Example 14

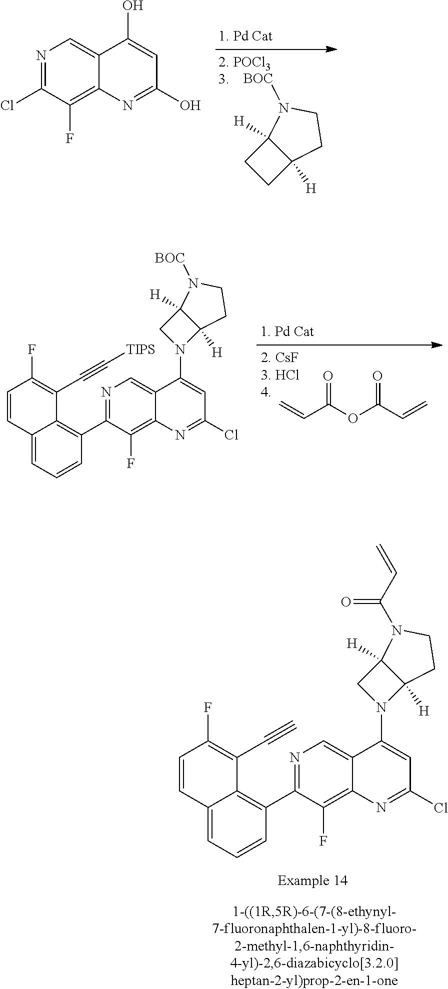

A mixture of 7-chloro-8-fluoro-1,6-naphthyridine-2,4-diol (3.00 g, 14.0 mmol, 1.0 equiv), ((2-fluoro-8-(4,4,5,5-tetramethyl-1,3,2-dioxaborolan-2-yl)naphthalen-1-yl)ethynyl)triisopropylsilane (6.96 g, 15.4 mmol, 1.1 equiv), [2-(2-aminophenyl)phenyl]palladium(1+);bis(1-adamantyl)-butyl-phosphane; methanesulfonate (1.02 g, 1.40 mmol, 0.1 equiv) and K₃PO₄ (1.5 M in H₂O, 28.0 mL, 3.0 equiv) in EtOH (100 mL) was stirred at 80° C. for 0.5 h. Subsequently, the mixture was concentrated under vacuum. The resultant residue was diluted with ethyl acetate (50 mL), washed with water (30 mL) and brine (30 mL), dried over anh Na₂SO₄, filtered and concentrated under vacuum. The crude material was purified by column chromatography (SiO₂, dichloromethane/methanol, 10:1, Rf=0.2) to give 8-fluoro-7-(7-fluoro-8-((triisopropylsilyl)ethynyl)naphthalen-1-yl)-1,6-naphthyridine-2,4-diol (2.6 g, 33% yield) as a yellow solid. LCMS [ESI, M+1]: 505.2.

A mixture of 8-fluoro-7-(7-fluoro-8-((triisopropylsilyl)ethynyl)naphthalen-1-yl)-1,6-naphthyridine-2,4-diol (400 mg, 793 μmol, 1.0 equiv), POCl₃ (972 mg, 6.34 mmol, 589 μL, 8.0 equiv) and DIEA (307 mg, 2.38 mmol, 414 μL, 3.0 equiv) in toluene (8 mL) was stirred at 110° C. for 1 h. The mixture was concentrated under vacuum. The residue was diluted with satd aq NaHCO₃ (2.0 mL) and extracted with ethyl acetate (2×5.0 mL). The combined organic layer was washed with brine (5.0 mL), dried over anh Na₂SO₄, filtered and concentrated under vacuum to give 2,4-dichloro-8-fluoro-7-(7-fluoro-8-((triisopropylsilyl)ethynyl)naphthalen-1-yl)-1,6-naphthyridine (440 mg, crude) as a yellow oil.

A mixture of 2,4-dichloro-8-fluoro-7-(7-fluoro-8-((triisopropylsilyl)ethynyl)naphthalen-1-yl)-1,6-naphthyridine (400 mg, crude), tert-butyl (1R,5R)-2,6-diazabicyclo[3.2.0]heptane-2-carboxylate (87.9 mg, 443 μmol) and DIEA (286 mg, 2.22 mmol, 386 μL) in DMSO (2.0 mL) was stirred at 40° C. for 0.5 h. The mixture was purified by reversed phase flash [water (0.1% FA)/acetonitrile]. The desired fractions were neutralized with NaHCO₃ (5.0 g) and the mixture was concentrated under vacuum to removed acetonitrile. The mixture was extracted with ethyl acetate (3×20 mL) and the combined organic layer was washed brine (30 mL), dried over anh Na₂SO₄, filtered and concentrated under vacuum to give tert-butyl (1R,5R)-6-(2-chloro-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (70 mg, 12% over two steps) as a yellow solid. ¹H NMR (400 MHz, CDCl₃-d): δ 9.16-9.10 (m, 1H), 7.97-7.87 (m, 2H), 7.61-7.53 (m, 2H), 7.38-7.30 (m, 1H), 6.36-6.29 (m, 1H), 5.27 (br t, J=5.6 Hz, 1H), 4.96-4.60 (m, 2H), 4.34-4.24 (m, 1H), 4.09-3.98 (m, 1H), 3.72-3.60 (m, 1H), 2.52-2.43 (m, 1H), 2.49-2.33 (m, 1H), 2.20-2.09 (m, 1H), 1.51 (s, 9H), 0.90-0.86 (m, 18H), 0.57-0.45 (m, 3H). LCMS [ESI, M+1]: 703.3.

A mixture of tert-butyl (1R,5R)-6-(2-chloro-8-fluoro-7-(7-fluoro-8-((triisopropylsilyl)ethynyl)naphthalen-1-yl)-1,6-naphthyridin-4-yl)-2,6-diazabicyclo[3.2.0]heptane-2-carboxylate (40 mg, 56.9 μmol, 1.0 equiv), methylboronic acid (34.0 mg, 569 μmol, 10 equiv), Pd(PPh₃)₄ (6.57 mg, 5.69 μmol, 0.1 equiv) and Cs₂CO₃ (55.6 mg, 171 μmol, 3.0 equiv) in dioxane (1 mL) and H₂O (0.3 mL) was stirred at 90° C. for 2 h. The mixture was concentrated and the residue was purified by reversed phase flash chromatography [water (0.1% FA)/acetonitrile] to give tert-butyl (1R,5R)-6-(8-fluoro-7-(7-fluoro-8-((triisopropylsilyl)ethynyl)naphthalen-1-yl)-2-methyl-1,6-naphthyridin-4-yl)-2,6-diazabicyclo[3.2.0]heptane-2-carboxylate (30 mg, 76% yield) as a yellow solid. LCMS [ESI, M+1]: 683.4.

A mixture of tert-butyl (1R,5R)-6-(8-fluoro-7-(7-fluoro-8-((triisopropylsilyl)ethynyl)naphthalen-1-yl)-2-methyl-1,6-naphthyridin-4-yl)-2,6-diazabicyclo[3.2.0]heptane-2-carboxylate (40 mg, 58.6 μmol, 1.0 equiv) and CsF (89.0 mg, 586 μmol, 21.6 μL, 10 equiv) in DMF (1.0 mL) was stirred at 40° C. for 2 h. The mixture was directly purified by reversed phase flash chromatography [water (0.1% FA)/acetonitrile] to give tert-butyl (1R,5R)-6-(7-(8-ethynyl-7-fluoronaphthalen-1-yl)-8-fluoro-2-methyl-1,6-naphthyridin-4-yl)-2,6-diazabicyclo[3.2.0]heptane-2-carboxylate (30 mg, 97% yield) as a yellow oil. LCMS [ESI, M+1]: 527.0.

A mixture of tert-butyl (1R,5R)-6-(7-(8-ethynyl-7-fluoronaphthalen-1-yl)-8-fluoro-2-methyl-1,6-naphthyridin-4-yl)-2,6-diazabicyclo[3.2.0]heptane-2-carboxylate (25 mg, 47.5 μmol, 1.0 equiv) in HCl/dioxane (4 M, 833 μL, 70.2 equiv) and MeCN (0.1 mL) was stirred at 0° C. for 0.5 h. The mixture was concentrated under vacuum to afford 4-((1R,5R)-2,6-diazabicyclo[3.2.0]heptan-6-yl)-7-(8-ethynyl-7-fluoronaphthalen-1-yl)-8-fluoro-2-methyl-1,6-naphthyridine (22 mg, crude, HCl) as a yellow oil. LCMS [ESI, M+1]: 427.1.

To a mixture of 4-((1R,5R)-2,6-diazabicyclo[3.2.0]heptan-6-yl)-7-(8-ethynyl-7-fluoronaphthalen-1-yl)-8-fluoro-2-methyl-1,6-naphthyridine (21 mg, crude, HCl salt) and DIEA (58.6 mg, 454 μmol, 79.0 μL) in dichloromethane (1.0 mL) was added prop-2-enoyl prop-2-enoate (5.72 mg, 45.4 μmol) at −40° C. The mixture was stirred at −40° C. for 30 mins. The mixture was diluted with dichloromethane (5.0 mL) and washed with brine (3.0 mL). The organic layer was dried over anh Na₂SO₄, filtered and concentrated under vacuum. The residue was purified by prep-HPLC [Phenomenex Gemini-NX C18 75×30 mm×3 μm; mobile phase, A: water (10 mM NH₄HCO₃), B: ACN; B %: 24%-54%; 8 min] and lyophilized to give 1-((1R,5R)-6-(7-(8-ethynyl-7-fluoronaphthalen-1-yl)-8-fluoro-2-methyl-1,6-naphthyridin-4-yl)-2,6-diazabicyclo[3.2.0]heptan-2-yl)prop-2-en-1-one (9.45 mg, 43% over two steps) as a yellow solid. ¹H NMR (400 MHz, CDCl₃): δ 9.12-9.01 (m, 1H), 8.04-7.92 (m, 2H), 7.66-7.51 (m, 2H), 7.34 (br t, J=8.8 Hz, 1H), 6.63-6.26 (m, 3H), 5.88-5.75 (m, 1H), 5.59-5.34 (m, 1H), 5.23-4.68 (m, 2H), 4.55-4.14 (m, 2H), 4.05-3.73 (m, 1H), 3.01-2.82 (m, 1H), 2.78-2.70 (m, 3H), 2.63-2.30 (m, 2H). LCMS [ESI, M+1]: 481.2.

### Example 15

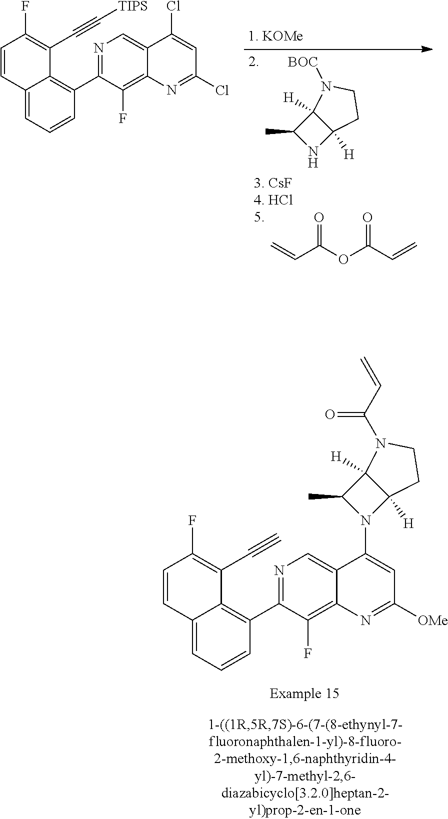

To a solution of methanol (160 mg, 4.99 mmol, 202 μL, 10 equiv) in toluene (3.00 mL) was added 4 Å MS (300 mg) and t-BuONa (144 mg, 1.50 mmol, 3.0 equiv) at 0° C. The mixture was stirred at 0° C. for 10 minutes. To this mixture was added 2,4-dichloro-8-fluoro-7-(7-fluoro-8-((triisopropylsilyl)ethynyl)naphthalen-1-yl)-1,6-naphthyridine (270 mg, 499 μmol, 1.0 equiv) at 0° C. and the mixture was stirred at 25° C. for 5 minutes. The reaction mixture was concentrated under reduced pressure to dryness. The residue was purified by column chromatography (SiO₂, petroleum ether/ethyl acetate, 50:1 to 5:1) to afford 4-chloro-8-fluoro-7-(7-fluoro-8-((triisopropylsilyl)ethynyl)naphthalen-1-yl)-2-methoxy-1,6-naphthyridine (140 mg, 51% yield) as a yellow solid. ¹H NMR (400 MHz, CDCl₃): δ 9.30 (s, 1H), 8.02-7.86 (m, 2H), 7.65-7.52 (m, 2H), 7.35 (t, J=8.8 Hz, 1H), 7.14 (s, 1H), 4.14 (s, 3H), 0.88 (d, J=7.6 Hz, 9H), 0.83 (d, J=7.6 Hz, 9H), 0.48 (quin, J=7.6 Hz, 3H). LCMS [ESI, M+1]: 537.2.

To a solution of 4-chloro-8-fluoro-7-(7-fluoro-8-((triisopropylsilyl)ethynyl)naphthalen-1-yl)-2-methoxy-1,6-naphthyridine (130 mg, 242 μmol, 1.0 equiv) and tert-butyl (1R,5R,7S)-7-methyl-2,6-diazabicyclo[3.2.0]heptane-2-carboxylate (77.1 mg, 363 μmol, 1.5 equiv) in 1,4-dioxane (3.00 mL) was added Cs₂CO₃ (237 mg, 726 μmol, 3.0 equiv), Pd(OAc)₂ (5.43 mg, 24.2 μmol, 0.10 equiv) and BINAP (30.1 mg, 48.4 μmol, 0.20 equiv). The mixture was stirred at 80° C. for 10 h. The mixture was filtered through a pad of SiO₂ and washed with ethyl acetate (30 mL). The filtrate was concentrated under reduced pressure to dryness. The residue was purified by column chromatography (SiO₂, petroleum ether/ethyl acetate, 10:1 to 2:1) to afford tert-butyl (1R,5R,7S)-6-(8-fluoro-7-(7-fluoro-8-((triisopropylsilyl)ethynyl)naphthalen-1-yl)-2-methoxy-1,6-naphthyridin-4-yl)-7-methyl-2,6-diazabicyclo[3.2.0]heptane-2-carboxylate (160 mg, 86% yield) as a yellow solid. ¹H NMR (400 MHz, CDCl₃): δ 9.15-9.05 (m, 1H), 7.99-7.87 (m, 2H), 7.63-7.50 (m, 3H), 7.33 (br t, J=8.8 Hz, 1H), 6.02-5.92 (m, 1H), 5.36-5.15 (m, 1H), 4.93-4.70 (m, 1H), 4.61-4.48 (m, 1H), 4.07 (d, J=2.0 Hz, 3H), 3.72-3.56 (m, 1H), 2.67-2.44 (m, 1H), 2.28-2.11 (m, 1H), 1.51 (br d, J=3.6 Hz, 9H), 1.47-1.40 (m, 3H), 0.91-0.84 (m, 18H), 0.59-0.45 (m, 3H). LCMS [ESI, M+1]: 713.8.

To a solution of tert-butyl (1R,5R,7S)-6-(8-fluoro-7-(7-fluoro-8-((triisopropylsilyl)ethynyl)naphthalen-1-yl)-2-methoxy-1,6-naphthyridin-4-yl)-7-methyl-2,6-diazabicyclo[3.2.0]heptane-2-carboxylate (150 mg, 210 μmol, 1.0 equiv) in DMF (2.00 mL) was added CsF (320 mg, 2.10 mmol, 10 equiv). The mixture was stirred at 25° C. for 1 h. The reaction mixture was concentrated under reduced pressure to dryness. The crude product was purified by reversed-phase flash (0.1% FA in water/ACN) to afford tert-butyl (1R,5R,7S)-6-(7-(8-ethynyl-7-fluoronaphthalen-1-yl)-8-fluoro-2-methoxy-1,6-naphthyridin-4-yl)-7-methyl-2,6-diazabicyclo[3.2.0]heptane-2-carboxylate (110 mg, 94% yield) as a yellow solid. ¹H NMR (400 MHz, CDCl₃): δ 9.10 (br d, J=6.4 Hz, 1H), 8.03-7.91 (m, 2H), 7.67-7.56 (m, 2H), 7.34 (t, J=8.8 Hz, 1H), 5.96 (d, J=3.6 Hz, 1H), 5.25 (q, J=6.0 Hz, 1H), 4.96-4.70 (m, 1H), 4.68-4.52 (m, 1H), 4.10 (s, 3H), 4.08-4.00 (m, 1H), 3.73-3.59 (m, 1H), 2.88-2.78 (m, 1H), 2.65-2.49 (m, 1H), 2.36-2.17 (m, 1H), 1.51 (br d, J=4.8 Hz, 9H), 1.48-1.42 (m, 3H). LCMS [ESI, M+1]: 557.2.

To a solution of tert-butyl (1R,5R,7S)-6-(7-(8-ethynyl-7-fluoronaphthalen-1-yl)-8-fluoro-2-methoxy-1,6-naphthyridin-4-yl)-7-methyl-2,6-diazabicyclo[3.2.0]heptane-2-carboxylate (100 mg, 180 μmol, 1.0 equiv) in MeCN (1.00 mL) was added HCl·1,4-dioxane (4 M, 1.00 mL, 22 equiv) at 0° C. The mixture was stirred at 0° C. for 20 minutes. The reaction mixture was concentrated under reduced pressure to afford 7-(8-ethynyl-7-fluoronaphthalen-1-yl)-8-fluoro-2-methoxy-4-((1R,5R,7S)-7-methyl-2,6-diazabicyclo[3.2.0]heptan-6-yl)-1,6-naphthyridine (88.0 mg, crude, HCl salt) as a yellow solid. LCMS [ESI, M+1]: 457.4.

To a solution of 7-(8-ethynyl-7-fluoronaphthalen-1-yl)-8-fluoro-2-methoxy-4-((1R,5R,7S)-7-methyl-2,6-diazabicyclo[3.2.0]heptan-6-yl)-1,6-naphthyridine (88.0 mg, 179 μmol, 1.0 equiv, HCl) in dichloromethane (2.00 mL) was added DIEA (92.3 mg, 714 μmol, 124 μL, 4.0 equiv) and prop-2-enoyl prop-2-enoate (45.0 mg, 357 μmol, 2.0 equiv) at −40° C. The mixture was stirred at −40° C. for 5 minutes and was concentrated under reduced pressure to dryness. The residue was purified by column chromatography (Al₂O₃, petroleum ether/ethyl acetate, 5:1 to 1:1) and prep-HPLC [column: Waters Xbridge 150*25 mm*5 um; mobile phase, A: water (10 mM NH₄HCO₃), B: ACN; B %: 47%-77%; 10 min] to afford 1-((1R,5R,7S)-6-(7-(8-ethynyl-7-fluoronaphthalen-1-yl)-8-fluoro-2-methoxy-1,6-naphthyridin-4-yl)-7-methyl-2,6-diazabicyclo[3.2.0]heptan-2-yl)prop-2-en-1-one (23.2 mg, 25% over two steps) as a yellow solid. ¹H NMR (400 MHz, CDCl₃): δ 9.12-9.03 (m, 1H), 8.06-7.90 (m, 2H), 7.70-7.55 (m, 2H), 7.34 (t, J=8.8 Hz, 1H), 6.69-6.28 (m, 2H), 6.05-5.93 (m, 1H), 5.89-5.75 (m, 1H), 5.43-4.90 (m, 2H), 4.77-4.17 (m, 2H), 4.10 (s, 3H), 3.99-3.69 (m, 1H), 2.90-2.78 (m, 1H), 2.77-2.61 (m, 1H), 2.43-2.16 (m, 1H), 1.47-1.38 (m, 3H). LCMS [ESI, M+1]: 511.3.

### Example 16

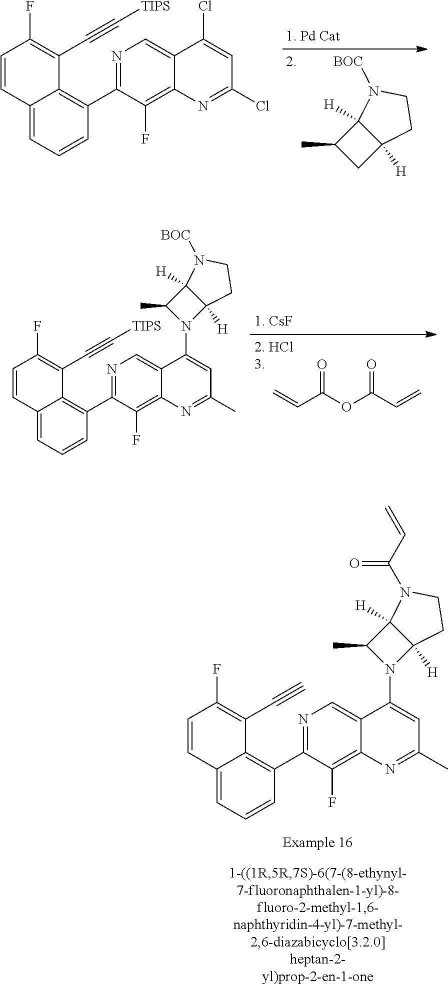

To a solution of 2,4-dichloro-8-fluoro-7-(7-fluoro-8-((triisopropylsilyl)ethynyl)naphthalen-1-yl)-1,6-naphthyridine (500 mg, 923 μmol, 1.00 equiv) and MeB(OH)₂ (166 mg, 2.77 mmol, 3.00 equiv) in THE (25.0 mL) was added Ad₂nBuP-Pd-G3 (67.2 mg, 92.3 μmol, 0.10 equiv) and K₃PO₄ (666 mg, 3.14 mmol, 3.40 equiv) in H₂O (5.00 mL). The mixture was stirred at 65° C. for 6 h. The reaction mixture was diluted with water (10 mL) and extracted with ethyl acetate (3×30 mL). The combined organic layer was dried over anhydrous sodium sulfate and filtered. The filtrate was concentrated under reduced pressure to afford 4-chloro-8-fluoro-7-(7-fluoro-8-((triisopropylsilyl)ethynyl)naphthalen-1-yl)-2-methyl-1,6-naphthyridine (80.0 mg, 16.6% yield) as a yellow solid. ¹H NMR (400 MHz, CDCl₃): δ 9.45 (s, 1H), 8.05-7.86 (m, 2H), 7.66-7.52 (m, 3H), 7.35 (t, J=8.8 Hz, 1H), 2.83 (s, 3H), 0.85 (d, J=7.6 Hz, 9H), 0.79 (d, J=7.6 Hz, 9H), 0.49-0.37 (m, 3H); LCMS [ESI, M+1]: 521.2.

To a solution of 4-chloro-8-fluoro-7-(7-fluoro-8-((triisopropylsilyl)ethynyl)naphthalen-1-yl)-2-methyl-1,6-naphthyridine (80.0 mg, 153 μmol, 1.00 equiv) and tert-butyl (1R,5R,7S)-7-methyl-2,6-diazabicyclo[3.2.0]heptane-2-carboxylate (65.18 mg, 307 μmol, 2.00 equiv) in dioxane (3.00 mL) was added Cs₂CO₃ (150 mg, 460 μmol, 3.00 equiv), Pd(OAc)₂ (3.45 mg, 15.3 μmol, 0.10 equiv) and BINAP (19.1 mg, 30.7 μmol, 0.20 equiv). The mixture was stirred at 80° C. for 10 h. The mixture was filtered through a pad of SiO₂ and washed with ethyl acetate (30 mL). The filtrate was concentrated under reduced pressure to dryness. The crude product was purified by reversed-phase flash (0.1% FA in water/ACN) to afford tert-butyl (1R,5R,7S)-6-(8-fluoro-7-(7-fluoro-8-((triisopropylsilyl)ethynyl)naphthalen-1-yl)-2-methyl-1,6-naphthyridin-4-yl)-7-methyl-2,6-diazabicyclo[3.2.0]heptane-2-carboxylate (105 mg, 79.5% yield) as a yellow solid. LCMS [ESI, M+1]: 697.4.

To a solution of tert-butyl (1R,5R,7S)-6-(8-fluoro-7-(7-fluoro-8-((triisopropylsilyl)ethynyl)naphthalen-1-yl)-2-methyl-1,6-naphthyridin-4-yl)-7-methyl-2,6-diazabicyclo[3.2.0]heptane-2-carboxylate (100 mg, 116 μmol, 1.00 equiv) in DMF (2.00 mL) was added CsF (176 mg, 1.16 mmol, 10.0 equiv). The mixture was stirred at 25° C. for 1 hour. The mixture was filtered and washed with MeCN (5.00 mL). The filtrate was concentrated under reduced pressure to dryness and the crude product was purified by reversed-phase flash (0.1% FA in water/ACN) to afford tert-butyl (1R,5R,7S)-6-(7-(8-ethynyl-7-fluoronaphthalen-1-yl)-8-fluoro-2-methyl-1,6-naphthyridin-4-yl)-7-methyl-2,6-diazabicyclo[3.2.0]heptane-2-carboxylate (60.0 mg, 95.5% yield) as a yellow solid. ¹H NMR (400 MHz, CDCl₃): δ 9.23-9.14 (m, 1H), 8.05-7.90 (m, 2H), 7.73-7.51 (m, 2H), 7.34 (t, J=8.8 Hz, 1H), 6.39 (d, J=2.8 Hz, 1H), 5.37-5.20 (m, 1H), 4.97-4.74 (m, 1H), 4.73-4.61 (m, 1H), 4.10 (s, 1H), 3.74-3.58 (m, 1H), 2.81-2.73 (m, 1H), 2.71 (d, J=1.6 Hz, 3H), 2.65-2.51 (m, 1H), 2.36-2.19 (m, 1H), 1.54-1.47 (m, 12H); LCMS [ESI, M+1]: 541.5.

To a solution of tert-butyl (1R,5R,7S)-6-(7-(8-ethynyl-7-fluoronaphthalen-1-yl)-8-fluoro-2-methyl-1,6-naphthyridin-4-yl)-7-methyl-2,6-diazabicyclo[3.2.0]heptane-2-carboxylate (55.0 mg, 102 μmol, 1.00 equiv) in ACN (1.00 mL) was added HCl·1,4-dioxane (4.00 M, 1.00 mL, 39.3 equiv) at 0° C. The mixture was stirred at 0° C. for 20 minutes and was concentrated under reduced pressure to afford 7-(8-ethynyl-7-fluoronaphthalen-1-yl)-8-fluoro-2-methyl-4-((1R,5R,7S)-7-methyl-2,6-diazabicyclo[3.2.0]heptan-6-yl)-1,6-naphthyridine (48 mg, crude, HCl salt) as a yellow solid. LCMS [ESI, M+1]: 441.3.

To a solution of 7-(8-ethynyl-7-fluoronaphthalen-1-yl)-8-fluoro-2-methyl-4-((1R,5R,7S)-7-methyl-2,6-diazabicyclo[3.2.0]heptan-6-yl)-1,6-naphthyridine (48.0 mg, 101 μmol, 1.00 equiv, HCl salt) in DCM (1.00 mL) was added DIEA (39.0 mg, 302 μmol, 52.6 μL, 3.00 equiv) and prop-2-enoyl prop-2-enoate (25.4 mg, 201 μmol, 2.00 equiv) at −40° C. The mixture was stirred at −40° C. for 5 minutes and was concentrated under reduced pressure. The residue was purified by column chromatography (Al₂O₃, petroleum ether/ethyl acetate, 5:1 to 0:1) and prep-HPLC (column: Phenomenex Gemini-NX C18 75*30 mm*3 μm; mobile phase, A: water (10 mM NH₄HCO₃), B: ACN; B %: 26%-56%; 8 min) to provide 1-((1R,5R,7S)-6-(7-(8-ethynyl-7-fluoronaphthalen-1-yl)-8-fluoro-2-methyl-1,6-naphthyridin-4-yl)-7-methyl-2,6-diazabicyclo[3.2.0]heptan-2-yl)prop-2-en-1-one (20.6 mg, 41.0% over two steps) as a yellow solid. ¹H NMR (400 MHz, CDCl₃): δ 9.25-9.11 (m, 1H), 8.04-7.90 (m, 2H), 7.71-7.54 (m, 2H), 7.34 (t, J=8.8 Hz, 1H), 6.69-6.30 (m, 3H), 5.90-5.77 (m, 1H), 5.53-4.92 (m, 2H), 4.87-4.68 (m, 1H), 4.61-4.14 (m, 1H), 4.02-3.67 (m, 1H), 2.90-2.61 (m, 5H), 2.52-2.18 (m, 1H), 1.53-1.43 (m, 3H). LCMS [ESI, M+1]: 495.2.

### Example 17

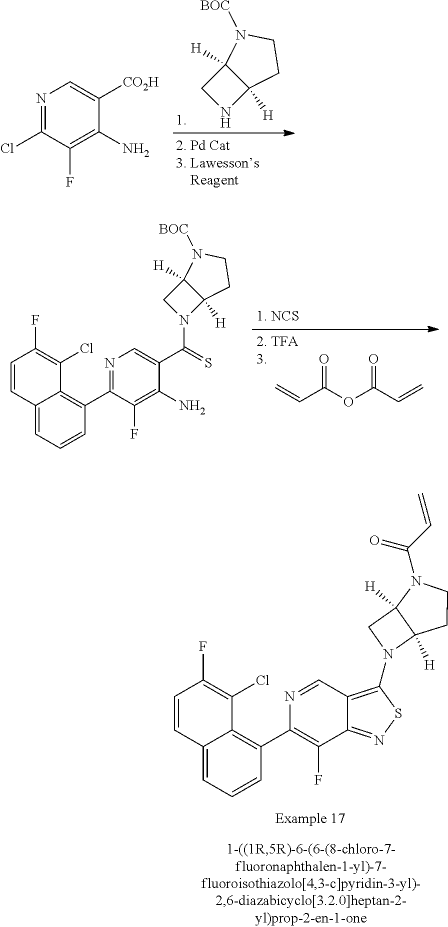

To a solution of 4-amino-6-chloro-5-fluoronicotinic acid (3 g, 15.7 mmol, 1 equiv) in DMF (30 mL) was added tert-butyl (1R,5R)-2,6-diazabicyclo[3.2.0]heptane-2-carboxylate (3.12 g, 15.7 mmol, 1 equiv), DIEA (6.10 g, 47.2 mmol, 8.23 mL, 3 equiv), 4 Å MS (3 g) and T3P (30 g, 47.2 mmol, 28.0 mL, 50% in DMF, 3 equiv). The mixture was stirred at 0° C. for 0.5 hour prior to being diluted water (100 mL) and adjusted to pH˜8 using NaHCO₃ solid. The aqueous layer was extracted with ethyl acetate (100 mL×3). The combined organic layer was dried over anhydrous sodium sulfate and filtered. The filtrate was concentrated under reduced pressure to dryness. The residue was purified by column chromatography (SiO₂, petroleum ether/ethyl acetate, 100:1 to 0:1) to provide tert-butyl (1R,5R)-6-(4-amino-6-chloro-5-fluoronicotinoyl)-2,6-diazabicyclo[3.2.0]heptane-2-carboxylate (3 g, 50% yield) as a white solid. LCMS [ESI, M+1]: 371.2.

To a solution of tert-butyl (1R,5R)-6-(4-amino-6-chloro-5-fluoronicotinoyl)-2,6-diazabicyclo[3.2.0]heptane-2-carboxylate (1 g, 2.70 mmol, 1 equiv) in toluene (15 mL) was added (8-chloro-7-fluoronaphthalen-1-yl)trimethylstannane (2.78 g, 8.09 mmol, 3 equiv), CuI (154 mg, 809 μmol, 0.3 equiv), BINAP (335 mg, 539 μmol, 0.2 equiv) and Pd(dppf)Cl₂ (197 mg, 269 μmol, 0.1 equi) under N₂. The mixture was stirred at 110° C. for 6 hours prior to being filtered and concentrated under reduced pressure. The residue was purified by column chromatography (SiO₂, petroleum ether/ethyl acetate, 100:1 to 0:1) to give tert-butyl (1R,5R)-6-(4-amino-6-(8-chloro-7-fluoronaphthalen-1-yl)-5-fluoronicotinoyl)-2,6-diazabicyclo[3.2.0]heptane-2-carboxylate (900 mg, 60% yield) as a brown solid. LCMS [ESI, M+1]: 515.3.

A mixture of tert-butyl (1R,5R)-6-(4-amino-6-(8-chloro-7-fluoronaphthalen-1-yl)-5-fluoronicotinoyl)-2,6-diazabicyclo[3.2.0]heptane-2-carboxylate (400 mg, 776 μmol, 1 equiv) and Lawesson's reagent (314 mg, 776 μmol, 1 equiv) in toluene (5 mL) was stirred at 80° C. for 4 hours prior to being concentrated under reduced pressure to give a residue. The residue was purified by column chromatography (SiO₂, petroleum ether/ethyl acetate, 100:1 to 0:1) to afford tert-butyl (1R,5R)-6-(4-amino-6-(8-chloro-7-fluoronaphthalen-1-yl)-5-fluoropyridine-3-carbonothioyl)-2,6-diazabicyclo[3.2.0]heptane-2-carboxylate (400 mg, 90% yield) as a yellow solid. LCMS [ESI, M+1]: 531.3.

To a solution of tert-butyl (1R,5R)-6-(4-amino-6-(8-chloro-7-fluoronaphthalen-1-yl)-5-fluoropyridine-3-carbonothioyl)-2,6-diazabicyclo[3.2.0]heptane-2-carboxylate (350 mg, 659 μmol, 1 equiv) in THE (1 mL) was added NCS (96.8 mg, 725 μmol, 1.1 equiv). The mixture was stirred at 40° C. for 0.5 hour prior to being concentrated under reduced pressure to give a residue. The residue was purified by column chromatography (SiO₂, petroleum ether/ethyl acetate, 100:1 to 0:1) to provide tert-butyl (1R,5R)-6-(6-(8-chloro-7-fluoronaphthalen-1-yl)-7-fluoroisothiazolo[4,3-c]pyridin-3-yl)-2,6-diazabicyclo[3.2.0]heptane-2-carboxylate (300 mg, 77% yield) as a yellow solid.

To a solution of tert-butyl (1R,5R)-6-(6-(8-chloro-7-fluoronaphthalen-1-yl)-7-fluoroisothiazolo[4,3-c]pyridin-3-yl)-2,6-diazabicyclo[3.2.0]heptane-2-carboxylate (300 mg, 567 μmol, 1 equiv) in DCM (5 mL) was added TFA (5.17 g, 45.3 mmol, 3.36 mL, 80 equiv). The mixture was stirred at 10° C. for 1 hour prior to being diluted with water (30 mL) and adjusted to pH 8 using solid Na₂CO₃. The mixture was extracted with ethyl acetate (30 mL×3). The combined organic layer was dried over anhydrous sodium sulfate and filtered and concentrated in vacuum to afford 3-((1R,5R)-2,6-diazabicyclo[3.2.0]heptan-6-yl)-6-(8-chloro-7-fluoronaphthalen-1-yl)-7-fluoroisothiazolo[4,3-c]pyridine (230 mg, crude) as a yellow solid.

To a solution of 3-((1R,5R)-2,6-diazabicyclo[3.2.0]heptan-6-yl)-6-(8-chloro-7-fluoronaphthalen-1-yl)-7-fluoroisothiazolo[4,3-c]pyridine (200 mg, 466 μmol, 1 equiv) in DCM (2 mL) was added TEA (141 mg, 1.40 mmol, 194 μL, 3 equiv) and acrylic anhydride (176 mg, 1.40 mmol, 3 equiv). The mixture was stirred at −40° C. for 0.5 h prior to being diluted with water (30 mL) and extracted with ethyl acetate (30 mL×3). The combined organic layer was dried over anhydrous sodium sulfate, filtered and concentrated in vacuum. The residue was purified by prep-HPLC [column: Waters Xbridge 150×25 mm×5 μm; mobile phase, A: water (10 mM NH₄HCO₃), B: ACN; B %: 39%-69%; 10 min] to afford 1-((1R,5R)-6-(6-(8-chloro-7-fluoronaphthalen-1-yl)-7-fluoroisothiazolo[4,3-c]pyridin-3-yl)-2,6-diazabicyclo[3.2.0]heptan-2-yl)prop-2-en-1-one (64.5 mg, 29% yield) as a yellow solid. ¹H NMR (400 MHz, CDCl₃): δ 8.71 (s, 1H), 7.97 (d, J=7.6 Hz, 1H), 7.90-7.83 (m, 1H), 7.65-7.54 (m, 2H), 7.38 (t, J=8.8 Hz, 1H), 6.63-6.33 (m, 2H), 5.87-5.80 (m, 1H), 5.50-5.00 (m, 2H), 4.79-4.47 (m, 1H), 4.34-4.18 (m, 2H), 4.08-3.76 (m, 1H), 2.70-2.50 (m, 1H), 2.30-2.03 (m, 1H). LCMS [ESI, M+1]: 483.1.

### Example 18

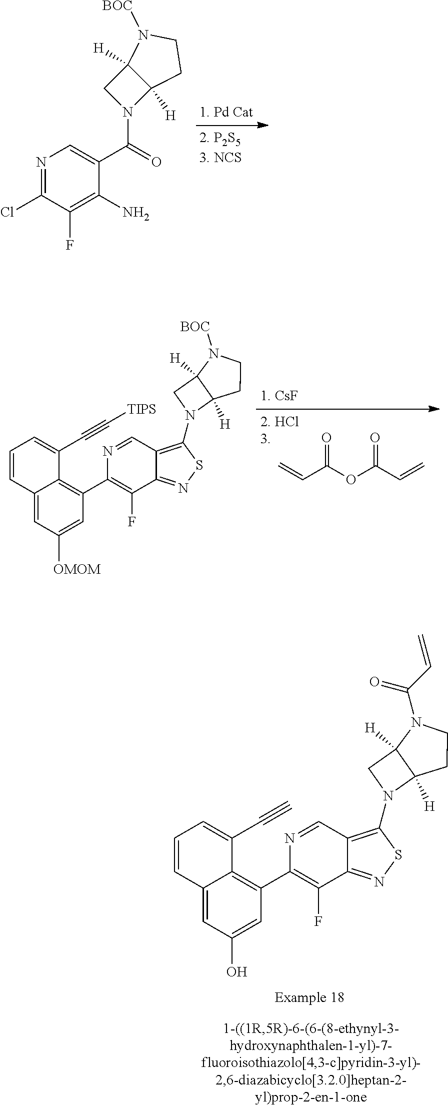

To a solution of tert-butyl (1R,5R)-6-(4-amino-6-chloro-5-fluoronicotinoyl)-2,6-diazabicyclo[3.2.0]heptane-2-carboxylate (1.00 g, 2.70 mmol, 1.0 equiv) and triisopropyl((6-(methoxymethoxy)-8-(4,4,5,5-tetramethyl-1,3,2-dioxaborolan-2-yl)naphthalen-1-yl)ethynyl)silane (1.60 g, 3.24 mmol, 1.2 equiv) in t-BuOH (15.0 mL) was added K₃PO₄ (1.5 M, 5.39 mL, 3.0 equiv) and SPhos Pd G3 (210 mg, 270 μmol, 0.1 equiv). The mixture was stirred at 80° C. for 16 h prior to being concentrated under reduced pressure to dryness. The crude product was purified by reversed-phase flash chromatography (0.1% FA in water/ACN) to provide tert-butyl (1R,5R)-6-(4-amino-5-fluoro-6-(3-(methoxymethoxy)-8-((triisopropylsilyl)ethynyl)naphthalen-1-yl)nicotinoyl)-2,6-diazabicyclo[3.2.0]heptane-2-carboxylate (1.00 g, 49% yield) as a yellow solid. ¹H NMR (400 MHz, CDCl₃): δ 8.36-8.26 (m, 1H), 7.78 (dd, J=1.2, 8.4 Hz, 1H), 7.71-7.66 (m, 1H), 7.48 (d, J=2.8 Hz, 1H), 7.39 (t, J=7.6 Hz, 1H), 7.26 (d, J=2.4 Hz, 1H), 6.72-5.93 (m, 2H), 5.44-5.06 (m, 3H), 4.72-4.42 (m, 2H), 4.12-4.03 (m, 1H), 4.00-3.88 (m, 1H), 3.70-3.54 (m, 1H), 3.53-3.47 (m, 3H), 2.64-2.18 (m, 1H), 2.05-1.93 (m, 1H), 1.47 (br s, 9H), 1.04-0.90 (m, 18H). LCMS [ESI, M+1]: 703.

A mixture of tert-butyl (1R,5R)-6-(4-amino-5-fluoro-6-(3-(methoxymethoxy)-8-((triisopropylsilyl)ethynyl)naphthalen-1-yl)nicotinoyl)-2,6-diazabicyclo[3.2.0]heptane-2-carboxylate (1.00 g, 1.42 mmol, 1.0 equiv) and P₂S₅ (3.16 g, 14.2 mmol, 10.0 equiv) in pyridine (15.0 mL) was purged with N₂ and stirred at 50° C. for 1 hour. The reaction mixture was diluted with water (70 mL) and ethyl acetate (70 mL), the water layer was adjusted to pH 5 with HCl aqueous solution (70 mL, 0.5 M). The mixture was extracted with ethyl acetate (3×30 mL). The combined organic layer was dried over anhydrous sodium sulfate, filtered and concentrated to provide a residue. The residue was purified by reversed-phase flash chromatography (0.1% FA in water/ACN) to give tert-butyl (1R,5R)-6-(4-amino-5-fluoro-6-(3-(methoxymethoxy)-8-((triisopropylsilyl)ethynyl)naphthalen-1-yl)pyridine-3-carbonothioyl)-2,6-diazabicyclo[3.2.0]heptane-2-carboxylate (770 mg, 71% yield) as a white solid. LCMS [ESI, M+1]: 719.

To a solution of tert-butyl (1R,5R)-6-(4-amino-5-fluoro-6-(3-(methoxymethoxy)-8-((triisopropylsilyl)ethynyl)naphthalen-1-yl)pyridine-3-carbonothioyl)-2,6-diazabicyclo[3.2.0]heptane-2-carboxylate (760 mg, 1.06 mmol, 1.0 equiv) in THF (7.00 mL) was added NCS (155 mg, 1.16 mmol, 1.1 equiv). The mixture was stirred at 40° C. for 0.5 hour prior to being concentrated under reduced pressure to dryness. The residue was purified by column chromatography (SiO₂, petroleum ether/ethyl acetate, 10:1 to 1:1) to give tert-butyl (1R,5R)-6-(7-fluoro-6-(3-(methoxymethoxy)-8-((triisopropylsilyl)ethynyl)naphthalen-1-yl)isothiazolo[4,3-c]pyridin-3-yl)-2,6-diazabicyclo[3.2.0]heptane-2-carboxylate (480 mg, 58% yield) as a yellow solid. LCMS [ESI, M+1]: 717.

To a solution of tert-butyl (1R,5R)-6-(7-fluoro-6-(3-(methoxymethoxy)-8-((triisopropylsilyl)ethynyl)naphthalen-1-yl)isothiazolo[4,3-c]pyridin-3-yl)-2,6-diazabicyclo[3.2.0]heptane-2-carboxylate (470 mg, 655 μmol, 1.0 equiv) in DMF (5.00 mL) was added CsF (797 mg, 5.24 mmol, 8.0 equiv). The mixture was stirred at 20° C. for 0.5 h prior to being diluted with water (5 mL) and extracted with ethyl acetate (10 mL). The combined organic layer was washed with brine (10 mL×3), dried over anhydrous sodium sulfate and filtered. The filtrate concentrated under reduced pressure to dryness. The residue was purified by column chromatography (SiO₂, petroleum ether/ethyl acetate, 10:1 to 1:1) to yield tert-butyl (1R,5R)-6-(6-(8-ethynyl-3-(methoxymethoxy)naphthalen-1-yl)-7-fluoroisothiazolo[4,3-c]pyridin-3-yl)-2,6-diazabicyclo[3.2.0]heptane-2-carboxylate (240 mg, 62% yield) as a yellow solid. ¹H NMR (400 MHz, CDCl₃): δ 8.71 (s, 1H), 7.86-7.80 (m, 1H), 7.62 (dd, J=1.2, 7.2 Hz, 1H), 7.52 (d, J=2.8 Hz, 1H), 7.44-7.36 (m, 1H), 7.35-7.31 (m, 1H), 5.40-5.28 (m, 2H), 5.23 (br t, J=5.2 Hz, 1H), 5.06-4.80 (m, 1H), 4.60 (br s, 1H), 4.27-4.17 (m, 1H), 4.12-3.99 (m, 1H), 3.77-3.64 (m, 1H), 3.53 (s, 3H), 2.55 (d, J=3.6 Hz, 1H), 2.47 (td, J=5.2, 14.0 Hz, 1H), 2.16-2.05 (m, 1H), 1.52 (s, 9H). LCMS [ESI, M+1]: 561.

To a solution of tert-butyl (1R,5R)-6-(6-(8-ethynyl-3-(methoxymethoxy)naphthalen-1-yl)-7-fluoroisothiazolo[4,3-c]pyridin-3-yl)-2,6-diazabicyclo[3.2.0]heptane-2-carboxylate (120 mg, 214 umol, 1.0 eq) in MeCN (1.0 mL) was added HCl·1,4-dioxane (4 M, 1.00 mL, 19.0 equiv). The mixture was stirred at 0° C. for 0.5 h prior to being concentrated. To the residue was added saturated NaHCO₃ (1.0 mL) dropwise to adjust pH 8˜9 at 0° C. The mixture was extracted with dichloromethane (3 mL×3). The combined organic layers were dried over anhydrous sodium sulfate and filtered. The filtrate was concentrated under reduced pressure to dryness to afford 4-(3-((1R,5R)-2,6-diazabicyclo[3.2.0]heptan-6-yl)-7-fluoroisothiazolo[4,3-c]pyridin-6-yl)-5-ethynylnaphthalen-2-ol (85.0 mg, 89% yield) as a yellow solid. LCMS [ESI, M+1]: 417.

To a solution of 4-(3-((1R,5R)-2,6-diazabicyclo[3.2.0]heptan-6-yl)-7-fluoroisothiazolo[4,3-c]pyridin-6-yl)-5-ethynylnaphthalen-2-ol (85.0 mg, 204 μmol, 1.0 equiv) in dichloromethane (0.500 mL) was added DIEA (79.1 mg, 612 μmol, 107 μL, 3.0 equiv) and prop-2-enoyl prop-2-enoate (25.7 mg, 204 μmol, 1.0 equiv). The mixture was stirred at −40° C. for 0.5 prior to being diluted with addition saturated NaHCO₃ (1.0 mL) at 0° C. The mixture was extracted with ethyl acetate (1 mL×3). The combined organic layer was dried over anhydrous sodium sulfate and filtered. The filtrate was concentrated under reduced pressure to dryness. The residue was purified by prep-HPLC [column: Phenomenex Gemini-NX C18 75×30 mm×3 μm; mobile phase, A: water (10 mM NH₄HCO₃), B: ACN; B %: 16%-46%; 8 min) to yield 1-((1R,5R)-6-(6-(8-ethynyl-3-hydroxynaphthalen-1-yl)-7-fluoroisothiazolo[4,3-c]pyridin-3-yl)-2,6-diazabicyclo[3.2.0]heptan-2-yl)prop-2-en-1-one (18.7 mg, 19% yield) as a yellow solid. ¹H NMR (400 MHz, CDCl₃): δ 8.70-8.59 (m, 1H), 7.64-7.56 (m, 1H), 7.51 (d, J=6.4 Hz, 1H), 7.32-7.27 (m, 1H), 7.13 (br d, J=8.8 Hz, 1H), 7.04 (br s, 1H), 6.58-6.17 (m, 2H), 5.86-5.73 (m, 1H), 5.34-4.78 (m, 2H), 4.59 (br dd, J=5.6, 9.2 Hz, 1H), 4.46-4.03 (m, 2H), 3.97-3.63 (m, 1H), 2.59-2.37 (m, 2H), 2.20-1.91 (m, 1H). LCMS [ESI, M+1]: 471.

### Example 19

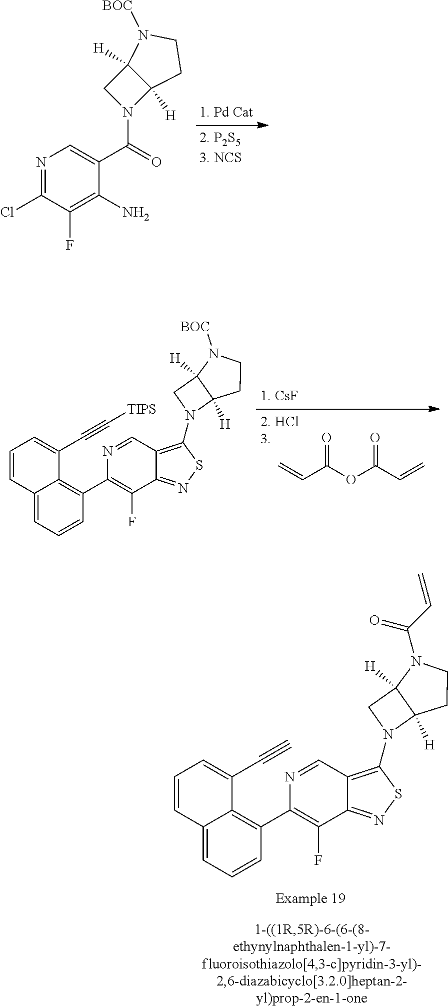

To a solution of tert-butyl (1R,5R)-6-(4-amino-6-chloro-5-fluoronicotinoyl)-2,6-diazabicyclo[3.2.0]heptane-2-carboxylate (1.00 g, 2.70 mmol, 1.0 equiv) and triisopropyl((8-(4,4,5,5-tetramethyl-1,3,2-dioxaborolan-2-yl)naphthalen-1-yl)ethynyl)silane (1.41 g, 3.24 mmol, 1.2 equiv) in t-BuOH (16.0 mL) was added K₃PO₄ (1.5 M, 5.39 mL, 3.0 equiv) and SPhos Pd G3 (210 mg, 270 μmol, 0.10 equiv). The mixture was stirred at 80° C. for 2 h prior to being concentrated under reduced pressure to dryness. The crude product was purified by reversed-phase flash chromatography (0.1% FA in water/ACN) to provide tert-butyl (1R,5R)-6-(4-amino-5-fluoro-6-(8-((triisopropylsilyl)ethynyl)naphthalen-1-yl)nicotinoyl)-2,6-diazabicyclo[3.2.0]heptane-2-carboxylate (720 mg, 41% yield) as a yellow solid. ¹H NMR (400 MHz, CDCl₃): δ 8.36-8.28 (m, 1H), 7.92 (ddd, J=1.2, 8.0, 14.0 Hz, 2H), 7.86-7.79 (m, 1H), 7.58-7.48 (m, 2H), 7.45 (t, J=7.6 Hz, 1H), 6.62-5.95 (m, 2H), 5.42-5.08 (m, 1H), 4.80-4.35 (m, 2H), 4.12-3.87 (m, 2H), 3.78-3.46 (m, 1H), 2.65-2.16 (m, 1H), 2.04 (br s, 1H), 1.47 (br s, 9H), 1.05-0.92 (m, 18H). LCMS [ESI, M+1]: 643.

A mixture of tert-butyl (1R,5R)-6-(4-amino-5-fluoro-6-(8-((triisopropylsilyl)ethynyl)naphthalen-1-yl)nicotinoyl)-2,6-diazabicyclo[3.2.0]heptane-2-carboxylate (660 mg, 1.03 mmol, 1.0 equiv) and P₂S₅ (2.28 g, 10.3 mmol, 10 equiv) in pyridine (10.0 mL) was flushed with N₂ and stirred at 50° C. for 1 h. Subsequently, the reaction mixture was diluted with water (50 mL), ethyl acetate (50 mL) and aq HCl (50 mL, 0.5 M). The aqueous layer was separated and further extracted with ethyl acetate (3×30 mL). The combined organic layer was dried over anhydrous sodium sulfate, filtered and concentrated to afford the crude material. The crude product was purified by reversed-phase flash chromatography (0.1% FA in water/ACN) to give tert-butyl (1R,5R)-6-(4-amino-5-fluoro-6-(8-((triisopropylsilyl)ethynyl)naphthalen-1-yl)pyridine-3-carbonothioyl)-2,6-diazabicyclo[3.2.0]heptane-2-carboxylate (460 mg, 65% yield) as a yellow solid. LCMS [ESI, M+1]: 659.

To a solution of tert-butyl (1R,5R)-6-(4-amino-5-fluoro-6-(8-((triisopropylsilyl)ethynyl)naphthalen-1-yl)pyridine-3-carbonothioyl)-2,6-diazabicyclo[3.2.0]heptane-2-carboxylate (450 mg, 683 μmol, 1.0 equiv) in THE (5.0 mL) was added NCS (100 mg, 751 μmol, 1.1 equiv). The mixture was stirred at 40° C. for 0.5 h prior to being concentrated under reduced pressure. The residue was purified by column chromatography (SiO₂, petroleum ether/ethyl acetate, 10:1 to 0:1) to yield tert-butyl (1R,5R)-6-(7-fluoro-6-(8-((triisopropylsilyl)ethynyl)naphthalen-1-yl)isothiazolo[4,3-c]pyridin-3-yl)-2,6-diazabicyclo[3.2.0]heptane-2-carboxylate (330 mg, 71% yield) as a yellow solid. LCMS [ESI, M+1]: 657.

To a solution of tert-butyl (1R,5R)-6-(7-fluoro-6-(8-((triisopropylsilyl)ethynyl)naphthalen-1-yl)isothiazolo[4,3-c]pyridin-3-yl)-2,6-diazabicyclo[3.2.0]heptane-2-carboxylate (320 mg, 487 μmol, 1.0 equiv) in DMF (3.00 mL) was added CsF (592 mg, 3.90 mmol, 8.0 equiv). The mixture was stirred at 20° C. for 0.5 h prior to being diluted with water (5 mL) and extracted with ethyl acetate (3×10 mL). The combined organic layer was washed with brine (10 mL×3), dried over anhydrous sodium sulfate, filtered and concentrated. The resultant residue was purified by column chromatography (SiO₂, petroleum ether/ethyl acetate, 10:1 to 1:1) to afford tert-butyl (1R,5R)-6-(6-(8-ethynylnaphthalen-1-yl)-7-fluoroisothiazolo[4,3-c]pyridin-3-yl)-2,6-diazabicyclo[3.2.0]heptane-2-carboxylate (240 mg, 95% yield) as a yellow solid. ¹H NMR (400 MHz, CDCl₃): δ 8.73 (s, 1H), 8.00-7.92 (m, 2H), 7.76 (dd, J=1.2, 7.2 Hz, 1H), 7.63-7.56 (m, 2H), 7.45 (dd, J=7.2, 8.0 Hz, 1H), 5.24 (br t, J=5.2 Hz, 1H), 5.08-4.79 (m, 1H), 4.60 (br s, 1H), 4.27-4.18 (m, 1H), 4.12 (s, 1H), 3.79-3.62 (m, 1H), 2.58 (d, J=3.6 Hz, 1H), 2.48 (td, J=5.2, 14.0 Hz, 1H), 2.11-2.06 (m, 1H), 1.52 (s, 9H). LCMS [ESI, M+1]: 501.

To a solution of tert-butyl (1R,5R)-6-(6-(8-ethynylnaphthalen-1-yl)-7-fluoroisothiazolo[4,3-c]pyridin-3-yl)-2,6-diazabicyclo[3.2.0]heptane-2-carboxylate (120 mg, 240 μmol, 1.0 equiv) in MeCN (1.00 mL) was added HCl·1,4-dioxane (4 M, 1 mL, 17.0 equiv) at 0° C. The mixture was stirred at 0° C. for 0.5 h prior to being concentrated. To the resultant residue was added saturated aq NaHCO₃ (1 mL) and the mixture was extracted with dichloromethane (3 mL×3). The combined organic layer was dried over anhydrous sodium sulfate and filtered. The filtrate was concentrated under reduced pressure to dryness to provide 3-((1R,5R)-2,6-diazabicyclo[3.2.0]heptan-6-yl)-6-(8-ethynylnaphthalen-1-yl)-7-fluoroisothiazolo[4,3-c]pyridine (90.0 mg, 79% yield) as a yellow solid. LCMS [ESI, M+1]: 401.

To a solution of 3-((1R,5R)-2,6-diazabicyclo[3.2.0]heptan-6-yl)-6-(8-ethynylnaphthalen-1-yl)-7-fluoroisothiazolo[4,3-c]pyridine (90.0 mg, 225 μmol, 1.0 equiv) in dichloromethane (0.500 mL) was added TEA (93.8 μL, 674 μmol, 3.0 equiv) and acrylic anhydride (34.0 mg, 270 μmol, 1.2 equiv). The mixture was stirred at −40° C. for 15 min prior to being diluted with saturated aq NaHCO₃ (1.0 mL) at 0° C. The mixture was extracted with ethyl acetate (1 mL×3). The combined organic layer was dried over anhydrous sodium sulfate, filtered and concentrated. The residue was purified by prep-HPLC [Column: Waters Xbridge 150×25 mm×5 μm; mobile phase, A: water (10 mM NH₄HCO₃), B: ACN), B %: 35%-65%; 9 min] to provide 1-((1R,5R)-6-(6-(8-ethynylnaphthalen-1-yl)-7-fluoroisothiazolo[4,3-c]pyridin-3-yl)-2,6-diazabicyclo[3.2.0]heptan-2-yl)prop-2-en-1-one (11.8 mg, 11% yield) as a yellow solid. ¹H NMR (400 MHz, CDCl₃): δ 8.89-8.80 (m, 1H), 8.04-7.92 (m, 2H), 7.76 (d, J=7.2 Hz, 1H), 7.63-7.51 (m, 2H), 7.46 (t, J=7.6 Hz, 1H), 6.64-6.28 (m, 2H), 5.84 (br d, J=8.0 Hz, 1H), 5.56-5.00 (m, 2H), 4.52 (br s, 1H), 4.39-4.14 (m, 2H), 4.10-3.70 (m, 1H), 2.77-2.52 (m, 2H), 2.33-2.24 (m, 1H). LCMS [ESI, M+1]: 455.

### Example 20

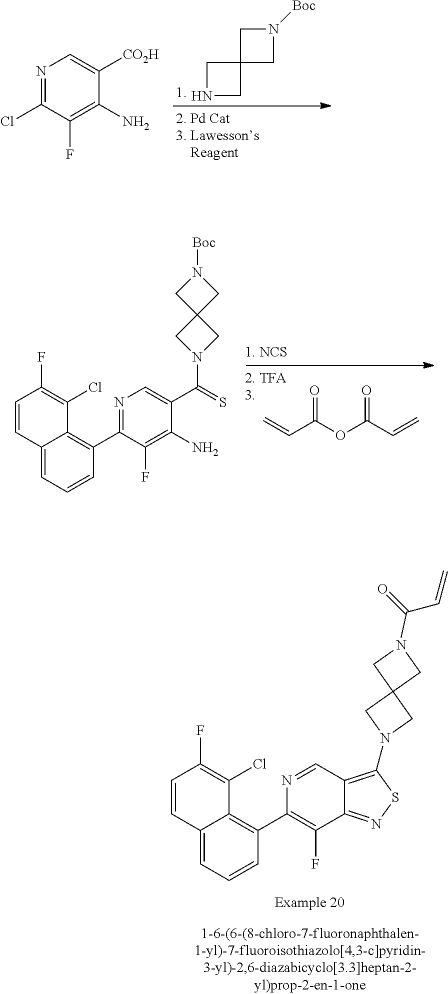

A mixture of 4-amino-6-chloro-5-fluoronicotinic acid (1 g, 5.25 mmol, 1.00 equiv), tert-butyl 2,6-diazaspiro[3.3]heptane-2-carboxylate-oxalate salt (2.27 g, 7.87 mmol, 1.5 equiv), T3P (9.36 mL, 15.7 mmol, 50% in EtOAc, 3.00 equiv), DIPEA (2.74 mL, 15.7 mmol, 3.00 equiv) in DMF (20 mL) was flushed with nitrogen. The mixture was stirred at 0° C. for 30 min prior to being diluted with satd aq NaHCO₃ (30 mL) at 5° C. and then H₂O (100 mL). The aqueous phase was extracted with EtOAc (3×100 mL). The combined organic layer was washed with brine (2×100 mL), dried over anh Na₂SO₄, filtered and concentrated under reduced pressure. The residue was purified by column chromatography (SiO₂, petroleum ether/ethyl acetate, 5:1 to 0:1) to afford tert-butyl 6-(4-amino-6-chloro-5-fluoronicotinoyl)-2,6-diazaspiro[3.3]heptane-2-carboxylate (720 mg, 35% yield) as a white solid. ¹H NMR (400 MHz, CDCl₃): δ 7.97 (s, 1H), 6.25 (br s, 2H), 4.39 (s, 4H), 4.11 (s, 4H), 1.45 (s, 9H). LCMS [ESI, M+1]: 371.1.

A mixture of tert-butyl 6-(4-amino-6-chloro-5-fluoronicotinoyl)-2,6-diazaspiro[3.3]heptane-2-carboxylate (668 mg, 1.8 mmol, 1 equiv), 8-chloro-7-fluoronaphthalen-1-yl)trimethylstannane (1.86 g, 5.42 mmol, 3.00 equiv), Pd(dppf)Cl₂ (132 mg, 181 μmol, 0.10 equiv), CuI (68.8 mg, 361 μmol, 0.20 equiv) and BINAP (225 mg, 361 μmol, 0.20 equiv) in toluene (8.00 mL) was flushed with N₂. The mixture was stirred at 110° C. for 6 h prior to being filtered and concentrated under vacuum to give a residue. The residue was purified by column chromatography (SiO₂, petroleum ether/ethyl acetate, 2:1 to 0:1) to give tert-butyl 6-(4-amino-6-(8-chloro-7-fluoronaphthalen-1-yl)-5-fluoronicotinoyl)-2,6-diazaspiro[3.3]heptane-2-carboxylate (660 mg, 71% yield) as a yellow solid. ¹H NMR (400 MHz, CDCl₃): δ 8.25 (s, 1H), 7.99-7.94 (m, 1H), 7.90-7.82 (m, 1H), 7.59-7.55 (m, 2H), 7.39 (t, J=8.8 Hz, 1H), 6.19 (br s, 2H), 4.62-4.32 (m, 4H), 4.14 (s, 4H), 1.46 (s, 9H).

To a solution of tert-butyl 6-(4-amino-6-(8-chloro-7-fluoronaphthalen-1-yl)-5-fluoronicotinoyl)-2,6-diazaspiro[3.3]heptane-2-carboxylate (660 mg, 1.28 mmol, 1.00 equiv) in toluene (10.0 mL) was added Lawesson's reagent (518 mg, 1.28 mmol, 1.00 equiv). The mixture was stirred at 80° C. for 4 h prior to being filtered and concentrated under vacuum to give a residue. The residue was purified by column chromatography (SiO₂, petroleum ether/ethyl acetate, 2:1 to 0:1) to afford tert-butyl 6-(4-amino-6-(8-chloro-7-fluoronaphthalen-1-yl)-5-fluoropyridine-3-carbonothioyl)-2,6-diazaspiro[3.3]heptane-2-carboxylate (400 mg, 59% yield) as a yellow solid. LCMS [ESI, M+1]: 530.9.

To a solution of 6-(4-amino-6-(8-chloro-7-fluoronaphthalen-1-yl)-5-fluoropyridine-3-carbonothioyl)-2,6-diazaspiro[3.3]heptane-2-carboxylate (350 mg, 659 μmol, 1.00 equiv) in THE (7.00 mL) was added NCS (96.8 mg, 725 μmol, 1.10 equiv). The mixture was stirred at 40° C. for 30 min prior to being diluted with water (20.0 mL) and extracted with ethyl acetate (3×20.0 mL). The combined organic layer was dried over anhydrous sodium sulfate and filtered. The filtrate was concentrated under reduced pressure to dryness. The crude product was purified by column chromatography (SiO₂, petroleum ether/ethyl acetate, 2:1 to 0:1) to give tert-butyl 6-(6-(8-chloro-7-fluoronaphthalen-1-yl)-7-fluoroisothiazolo[4,3-c]pyridin-3-yl)-2,6-diazaspiro[3.3]heptane-2-carboxylate (170 mg, 49% yield) as a yellow solid. ¹H NMR (400 MHz, CDCl₃): δ 8.77 (s, 1H), 7.99-7.96 (m, 1H), 7.89-7.85 (m, 1H), 7.64-7.54 (m, 2H), 7.38 (t, J=8.8 Hz, 1H), 4.68-4.59 (m, 4H), 4.26 (s, 4H), 1.47 (s, 9H). LCMS [ESI, M+1]: 528.9.

To a solution of tert-butyl 6-(6-(8-chloro-7-fluoronaphthalen-1-yl)-7-fluoroisothiazolo[4,3-c]pyridin-3-yl)-2,6-diazaspiro[3.3]heptane-2-carboxylate (60.0 mg, 113 μmol, 1.00 equiv) in DCM (1.00 mL) was added TFA (600 μL, 8.10 mmol, 71.4 equiv). The mixture was stirred at 0° C. for 1 h prior to being diluted with water (10.0 mL) and saturated aq sodium bicarbonate (5.00 mL). The aqueous phase was extracted with DCM (3×10.0 mL). The combined organic layer was dried over anhydrous sodium sulfate, filtered and concentrated at reduced pressure to provide 6-(8-chloro-7-fluoronaphthalen-1-yl)-7-fluoro-3-(2,6-diazaspiro[3.3]heptan-2-yl)isothiazolo[4,3-c]pyridine (40.0 mg, crude) as a yellow solid. ¹H NMR (400 MHz, CDCl₃): δ 8.69 (s, 1H), 7.96 (d, J=8.0 Hz, 1H), 7.90-7.84 (m, 1H), 7.63-7.55 (m, 2H), 7.38 (t, J=8.8 Hz, 1H), 4.62-4.54 (m, 4H), 3.96 (s, 4H). LCMS [ESI, M+1]: 429.0.

To a solution of 6-(8-chloro-7-fluoronaphthalen-1-yl)-7-fluoro-3-(2,6-diazaspiro[3.3]heptan-2-yl)isothiazolo[4,3-c]pyridine (40.0 mg, 93.3 μmol, 1.00 equiv) in DCM (1.00 mL) was added TEA (38.9 μL, 280 μmol, 3.00 equiv) and acrylic anhydride (35.3 mg, 280 μmol, 3.00 equiv). The mixture was stirred at −40° C. for 30 min prior to being diluted with water (10.0 mL) and extracted with ethyl acetate (3×10.0 mL). The combined organic layer was dried over anhydrous sodium sulfate, filtered and concentrated under reduced pressure. The residue was purified by prep-HPLC [Column: Waters Xbridge 150×25 mm×5 μm; mobile phase, A: water (10 mM NH₄HCO₃), B: ACN, B %: 30%-60%; 10 min] to afford 1-(6-(6-(8-chloro-7-fluoronaphthalen-1-yl)-7-fluoroisothiazolo[4,3-c]pyridin-3-yl)-2,6-diazaspiro[3.3]heptan-2-yl)prop-2-en-1-one (11.3 mg, 25% over two steps) as a yellow solid. ¹H NMR (400 MHz, CDCl₃): δ 8.70 (d, J=1.2 Hz, 1H), 7.99-7.94 (m, 1H), 7.89-7.83 (m, 1H), 7.64-7.54 (m, 2H), 7.38 (t, J=8.8 Hz, 1H), 6.43-6.35 (m, 1H), 6.25-6.15 (m, 1H), 5.78-5.74 (m, 1H), 4.70-4.62 (m, 4H), 4.56 (br s, 2H), 4.41 (br s, 2H). LCMS [ESI, M+1]: 482.9.

### Example 21

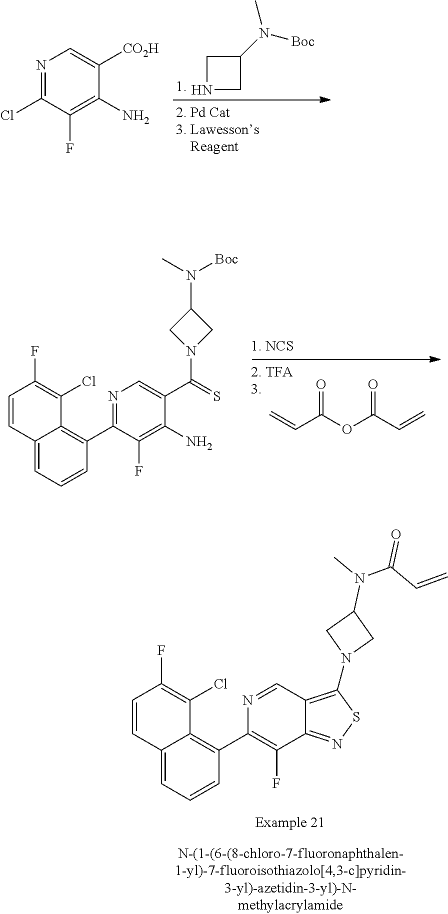

To a solution of 4-amino-6-chloro-5-fluoronicotinic acid (1.30 g, 6.82 mmol, 1.00 equiv) in DMF (20.0 mL) was added DIEA (3.56 mL, 20.5 mmol, 3.00 equiv), tert-butyl azetidin-3-yl(methyl)carbamate (2.16 g, 11.6 mmol, 1.70 equiv) and T3P (12.2 mL, 20.5 mmol, 50% in EtOAc, 3.00 equiv). The mixture was stirred at −40° C. for 30 min prior to being diluted with water (20.0 mL). The pH was adjusted to ˜ 8 using solid NaHCO₃ and the aqueous layer was extracted with ethyl acetate (3×20.0 mL). The combined organic layer was dried over anhydrous sodium sulfate, filtered and concentrated under reduced pressure to provide tert-butyl (1-(4-amino-6-chloro-5-fluoronicotinoyl)azetidin-3-yl)(methyl)carbamate (1.56 g, crude) as a white solid. ¹H NMR (400 MHz, CDCl₃): δ 8.00 (s, 1H), 6.45-6.04 (m, 2H), 4.97-4.77 (m, 1H), 4.58-4.17 (m, 4H), 2.94 (s, 3H), 1.47 (s, 9H). LCMS [ESI, M+1]: 359.1.

A mixture of tert-butyl (1-(4-amino-6-chloro-5-fluoronicotinoyl)azetidin-3-yl)(methyl)carbamate (670 mg, 1.87 mmol, 1.00 equiv), (8-chloro-7-fluoro-1-naphthyl)-trimethyl-stannane (1.80 g, 5.23 mmol, 2.80 equiv), CuI (71.1 mg, 374 μmol, 0.20 equiv), BINAP (232 mg, 373 μmol, 0.20 equiv) and Pd(dppf)Cl₂ (136 mg, 187 μmol, 0.10 equiv) in toluene (1.00 mL) was stirred at 110° C. for 6 hours under nitrogen. The reaction mixture was subsequently filtered and concentrated under vacuum to give a residue. The residue was purified by column chromatography (SiO₂, petroleum ether/ethyl acetate, 1:0 to 0:1) to provide tert-butyl (1-(4-amino-6-(8-chloro-7-fluoronaphthalen-1-yl)-5-fluoronicotinoyl)azetidin-3-yl)(methyl)carbamate (570 mg, 61% yield) as a yellow solid. ¹H NMR (400 MHz, CDCl₃): δ 8.28 (s, 1H), 7.98-7.93 (m, 1H), 7.89-7.82 (m, 1H), 7.59-7.55 (m, 2H), 7.38 (t, J=8.8 Hz, 1H), 6.19 (br s, 2H), 5.17-4.70 (m, 1H), 4.63-4.30 (m, 4H), 2.97 (s, 3H), 1.48 (s, 9H). LCMS [ESI, M+1]: 503.2.

To a solution of tert-butyl (1-(4-amino-6-(8-chloro-7-fluoronaphthalen-1-yl)-5-fluoronicotinoyl)azetidin-3-yl)(methyl)carbamate (520 mg, 1.03 mmol, 1.00 equiv) in toluene (20.0 mL) was added Lawesson's reagent (418 mg, 1.03 mmol, 1.00 equiv). The mixture was stirred at 80° C. for 4 h prior to being filtered and concentrated under vacuum to give a residue. The residue was purified by column chromatography (SiO₂, petroleum ether/ethyl acetate, 2:1 to 0:1) to give tert-butyl (1-(4-amino-6-(8-chloro-7-fluoronaphthalen-1-yl)-5-fluoropyridine-3-carbonothioyl)azetidin-3-yl)(methyl)carbamate (460 mg, 86% yield) as a yellow solid. LCMS [ESI, M+1]: 519.0.

To a solution of tert-butyl (1-(4-amino-6-(8-chloro-7-fluoronaphthalen-1-yl)-5-fluoropyridine-3-carbonothioyl)azetidin-3-yl)(methyl)carbamate (410 mg, 790 μmol, 1.00 equiv) in THF (8.00 mL) was added NCS (116 mg, 869 μmol, 1.10 equiv). The mixture was stirred at 40° C. for 30 min prior to being diluted with water (20.0 mL) and extracted with ethyl acetate (3×20.0 mL). The combined organic layer was dried over anhydrous sodium sulfate, filtered and concentrated under reduced pressure. The crude product was purified by column chromatography (SiO₂, petroleum ether/ethyl acetate, 2:1 to 0:1) to provide tert-butyl (1-(6-(8-chloro-7-fluoronaphthalen-1-yl)-7-fluoroisothiazolo[4,3-c]pyridin-3-yl)azetidin-3-yl)(methyl)carbamate (330 mg, 81% yield) as a yellow solid. ¹H NMR (400 MHz, CDCl₃): δ 8.74 (s, 1H), 7.96 (br d, J=7.6 Hz, 1H), 7.89-7.84 (m, 1H), 7.66-7.52 (m, 2H), 7.38 (t, J=8.8 Hz, 1H), 5.51-5.02 (m, 1H), 4.73-4.52 (m, 4H), 3.76-3.69 (m, 3H), 1.50 (s, 9H). LCMS [ESI, M+1]: 517.1.

To a solution of tert-butyl (1-(6-(8-chloro-7-fluoronaphthalen-1-yl)-7-fluoroisothiazolo[4,3-c]pyridin-3-yl)azetidin-3-yl)(methyl)carbamate (120 mg, 232 μmol, 1.00 equiv) in DCM (2.00 mL) was added TFA (1.00 mL, 13.5 mmol, 58.2 equi). The mixture was stirred at 0° C. for 1 h prior to being diluted with water (10.0 mL) (pH adjusted to ˜8 with solid Na₂CO₃). The aqueous phase was extracted with ethyl acetate (3×10.0 mL). The combined organic layer was dried over anhydrous sodium sulfate, filtered and concentrated under reduced pressure to afford 1-(6-(8-chloro-7-fluoronaphthalen-1-yl)-7-fluoroisothiazolo[4,3-c]pyridin-3-yl)-N-methylazetidin-3-amine (90.0 mg, crude) as a white solid.

To a solution of 1-(6-(8-chloro-7-fluoronaphthalen-1-yl)-7-fluoroisothiazolo[4,3-c]pyridin-3-yl)-N-methylazetidin-3-amine (80.0 mg, 192 μmol, 1.00 equiv) in DCM (2.00 mL) was added TEA (80.1 μL, 576 μmol, 3.00 equiv) and acrylic anhydride (72.6 mg, 576 μmol, 3.00 equiv). The mixture was stirred at −40° C. for 10 min prior to being diluted with water (10.0 mL) and extracted with ethyl acetate (3×10.0 mL). The combined organic layer was dried over anhydrous sodium sulfate, filtered and concentrated under reduced pressure. The crude product was purified by prep-HPLC [Column: Phenomenex Gemini-NX C18 75×30 mm×3 μm; mobile phase, A: water (10 mM NH₄HCO₃, B: ACN, B %: 26%-56%; 8 min] to afford N-(1-(6-(8-chloro-7-fluoronaphthalen-1-yl)-7-fluoroisothiazolo[4,3-c]pyridin-3-yl)azetidin-3-yl)-N-methylacrylamide (10.6 mg, 12% over two steps) as a yellow solid. ¹H NMR (400 MHz, CDCl₃): δ 8.72 (d, J=1.2 Hz, 1H), 7.98-7.94 (m, 1H), 7.89-7.84 (m, 1H), 7.64-7.54 (m, 2H), 7.38 (t, J=8.8 Hz, 1H), 6.65-6.55 (m, 1H), 6.42 (br d, J=16.8 Hz, 1H), 5.87-5.79 (m, 1H), 5.68-5.54 (m, 1H), 4.77-4.71 (m, 2H), 4.64-4.55 (m, 2H), 3.28 (s, 3H). LCMS [ESI, M+1]: 471.0.

### Example 22

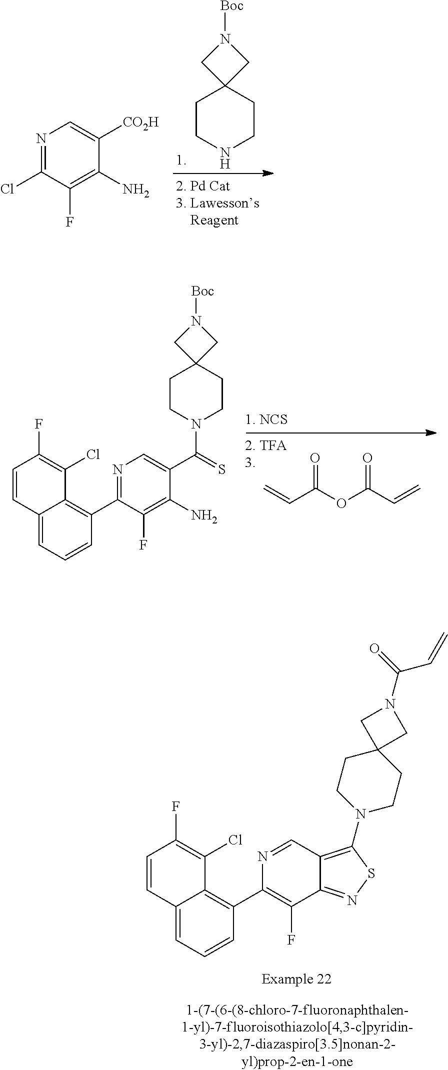

A mixture of 4-amino-6-chloro-5-fluoronicotinic acid (1.00 g, 5.25 mmol, 1.0 equiv), tert-butyl 2,7-diazaspiro[3.5]nonane-2-carboxylate (1.19 g, 5.25 mmol, 1.0 equiv), DIEA (2.03 g, 15.7 mmol, 3.0 equiv), T₃P (10.0 g, 15.7 mmol, 50% in EtOAc, 3.0 equiv) in DMF (20 mL) was stirred at 0° C. for 30 min under an atmosphere of nitrogen. The reaction mixture was subsequently diluted with sat aq NaHCO₃ (20 mL) at 5° C. and H₂O (50 mL). The aqueous phase was extracted with ethyl acetate (40 mL×2). The combined organic layer was washed with brine (25 mL×4), dried over anh Na₂SO₄, filtered and concentrated under reduced pressure to give a residue. The residue was purified by column chromatography (SiO₂, petroleum ether/ethyl acetate, 20:1 to 1:1) to afford tert-butyl 7-(4-amino-6-chloro-5-fluoronicotinoyl)-2,7-diazaspiro[3.5]nonane-2-carboxylate (1.30 g, 59% yield) as a white solid. ¹H NMR (400 MHz, CDCl₃): δ 7.84 (s, 1H), 5.33 (s, 2H), 3.71 (s, 2H), 3.58 (s, 2H), 2.97 (s, 2H), 2.90 (s, 2H), 1.82 (t, J=5.6 Hz, 4H), 1.46 (s, 9H). LCMS [ESI, M+1]: 399.

A mixture of tert-butyl 7-(4-amino-6-chloro-5-fluoronicotinoyl)-2,7-diazaspiro[3.5]nonane-2-carboxylate (1.00 g, 2.51 mmol, 1.0 equiv), (8-chloro-7-fluoro-1-naphthyl)-trimethyl-stannane (1.72 g, 5.01 mmol, 2.0 equiv), Pd(dppf)Cl₂ (183 mg, 250 μmol, 0.1 equiv), CuI (143 mg, 752 μmol, 0.3 equiv) and BINAP (312 mg, 501 μmol, 0.2 equiv) in toluene (10 mL) was stirred at 110° C. for 12 h under an atmosphere of nitrogen. The reaction mixture was subsequently concentrated under reduced pressure to give a residue. The residue was purified by column chromatography (SiO₂, petroleum ether/ethyl acetate, 10:1 to 0:1) to provide tert-butyl 7-(4-amino-6-(8-chloro-7-fluoronaphthalen-1-yl)-5-fluoronicotinoyl)-2,7-diazaspiro[3.5]nonane-2-carboxylate (1.30 g, 85% yield) as a brown solid. LCMS [ESI, M+1]: 543.0.

A mixture of tert-butyl 7-(4-amino-6-(8-chloro-7-fluoronaphthalen-1-yl)-5-fluoronicotinoyl)-2,7-diazaspiro[3.5]nonane-2-carboxylate (700 mg, 1.29 mmol, 1.0 equiv), Lawesson's reagent (521 mg, 1.29 mmol, 1.0 equiv) in toluene (10 mL) was stirred at 80° C. for 4 hours under an atmosphere of nitrogen. The reaction mixture was subsequently concentrated under reduced pressure to give a residue. The residue was purified by column chromatography (SiO₂, petroleum ether/ethyl acetate, 50:1 to 0:1) to provide tert-butyl 7-(4-amino-6-(8-chloro-7-fluoronaphthalen-1-yl)-5-fluoropyridine-3-carbonothioyl)-2,7-diazaspiro[3.5]nonane-2-carboxylate (420 mg, 53% yield) as a yellow solid. LCMS [ESI, M+1]: 559.

To a solution of tert-butyl 7-(4-amino-6-(8-chloro-7-fluoronaphthalen-1-yl)-5-fluoropyridine-3-carbonothioyl)-2,7-diazaspiro[3.5]nonane-2-carboxylate (220 mg, 393 μmol, 1.0 equiv) in THE (4.0 mL) was added NCS (57.8 mg, 433 μmol, 1.1 equiv) at 25° C. The reaction mixture was stirred at 25° C. for 2 h prior to being diluted with water (10 mL) and extracted with ethyl acetate (3×20 mL). The combined organic layer was dried over anh Na₂SO₄, filtered and concentrated under reduced pressure. The residue was purified by reversed phase flash chromatography (0.1% FA in water/ACN) to give tert-butyl 7-(6-(8-chloro-7-fluoronaphthalen-1-yl)-7-fluoroisothiazolo[4,3-c]pyridin-3-yl)-2,7-diazaspiro[3.5]nonane-2-carboxylate (55.0 mg, 82.9 μmol, 21% yield) as a yellow solid. LCMS [ESI, M+1]: 557.

To a solution of tert-butyl 7-(6-(8-chloro-7-fluoronaphthalen-1-yl)-7-fluoroisothiazolo[4,3-c]pyridin-3-yl)-2,7-diazaspiro[3.5]nonane-2-carboxylate (70.0 mg, 126 μmol, 1.0 equiv) in DCM (2.0 mL) was added TFA (2.0 mL) at 0° C. The reaction mixture was stirred at 0° C. for 0.5 hour. Subsequently, the reaction mixture was concentrated under reduced pressure to provide 6-(8-chloro-7-fluoronaphthalen-1-yl)-7-fluoro-3-(2,7-diazaspiro[3.5]nonan-7-yl)isothiazolo[4,3-c]pyridine (57.0 mg, crude) as a yellow solid.

To a solution of 6-(8-chloro-7-fluoronaphthalen-1-yl)-7-fluoro-3-(2,7-diazaspiro[3.5]nonan-7-yl)isothiazolo[4,3-c]pyridine (57.0 mg, 125 μmol, 1.0 equiv) and TEA (37.9 mg, 374 μmol, 3.0 equiv) in DCM (2.0 mL) was added acrylic anhydride (20.4 mg, 162 μmol, 1.3 equiv) at −40° C. The mixture was stirred at −40° C. for 0.5 hour. Subsequently, the mixture was diluted with water (20 mL) and extracted with ethyl acetate (3×20 mL). The combined organic layer was dried over anhydrous sodium sulfate, filtered and concentrated under reduced pressure. The residue was purified by prep-HPLC [Column: Phenomenex Gemini-NX C18 75×30 mm×3 μm; mobile phase, A: water (10 mM NH₄HCO₃), B: ACN; B %: 28%-58%; 8 min] to afford 1-(7-(6-(8-chloro-7-fluoronaphthalen-1-yl)-7-fluoroisothiazolo[4,3-c]pyridin-3-yl)-2,7-diazaspiro[3.5]nonan-2-yl)prop-2-en-1-one (20.2 mg, 30% yield,) as a yellow solid. ¹H NMR (400 MHz, CDCl₃): δ 9.00 (d, J=0.8 Hz, 1H), 7.97 (dd, J=8.0, 1.2 Hz, 1H), 7.87 (dd, J=9.2, 5.6 Hz, 1H), 7.66-7.54 (m, 2H), 7.38 (t, J=8.8 Hz, 1H), 6.46-6.35 (m, 1H), 6.29-6.17 (m, 1H), 5.74 (dd, J=10.4, 1.6 Hz, 1H), 4.06 (s, 2H), 3.95 (s, 2H), 3.82-3.65 (m, 4H), 2.15 (t, J=5.6 Hz, 4H). LCMS [ESI, M+1]: 511.

### Example A

### Inhibition of KRas G12C-Dependent Cell Growth

This Example illustrates that exemplary compounds of the present invention inhibit the growth of tumor cell lines that express KRas G12C.

The cellular inhibition of KRAs G12C by exemplary compounds of the present invention was determined by measuring the amount of a downstream marker of KRas activity, phosphorylated ERK (“Phospho-ERK”).

NCI-H358 cells (ATCC CRL-5807) express KRas G12C and were grown in RPMI medium supplemented with 10% fetal bovine serum, penicillin/streptomycin and 10 mM HEPES. Cells were plated in poly-D-Lysine coated 96-well plates at a concentration of 50,000 cells/well and allowed to attach for 8-12 hours. Diluted compounds were then added at a final concentration of 0.5% DMSO. After 3 hours, the medium was removed, 150 μL of 4% formaldehyde was added and the plates were incubated for 20 minutes. The plates were washed with PBS, and permeabilized using 150 μL of ice cold 100% methanol for 10 minutes. Non-specific antibody binding to the plates was blocked using 100 μL Licor Blocking Buffer (Li-Cor Biotechnology, Lincoln NE) for 1 hour at room temperature. Positive control samples and samples lacking cells were parallel processed with test samples as standards.

The amount Phospho-ERK was determined using an antibody specific for the phosphorylated form of ERK and compared to the amount of GAPDH. Primary antibodies used for detection were added as follows: Phospho-ERK (Cell Signaling cs9101) diluted 1:500 and GAPDH (Millipore MAB374) diluted 1:5000 in Licor block+0.05% Tween 20. The plates were incubated for 2 hours at room temperature. The plates were washed with PBS+0.05% Tween 20.

Secondary antibodies used to visualize primary antibodies were added as follows: Anti-rabbit-680 diluted 1:1000 and Anti-mouse-800 diluted 1:1000 in Licor Block+0.05% Tween 20 and incubated for 1 hour at room temperature. The plates were washed with PBS+0.05% Tween 20. A 100 μL aliquot of PBS was added to each well and the plates were read on a LICOR AERIUS plate reader.

The pERK(Thr202/Tyr204) signal was normalized with the GAPDH signal and percent of DMSO control values were calculated. IC₅₀ values were generated using a 4 parameter fit of the dose response curve. The results for exemplary compounds of Formula (I) are shown in Table 1.

TABLE 1
Inhibition of KRas G12C-mediated pERK
in H358 Cells by Exemplary Compounds

| Example No. | IC₅₀ (nM) |
|---|---|
| 1 | 2743 |
| 2 | 3050 |
| 3 | 75 |
| 4 | 169 |
| 5 | 5000 |
| 6 | 22 |
| 7 | 5913 |
| 8 | 1 |
| 9 | 9000 |
| 10 | 3675 |
| 11 | 17 |
| 12 | 24 |
| 13 | 31 |
| 14 | 21 |
| 15 | 1441 |
| 16 | 73 |
| 17 | 6000 |
| 18 | 14 |
| 19 | 748 |
| 20 | 7000 |
| 21 | 9000 |
| 22 | 8000 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of formula (I): 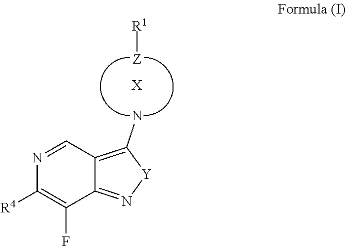
or a pharmaceutically acceptable salt thereof, wherein:
X is a 4-12 membered saturated or partially saturated monocyclic, bridged, spirocyclic or fused-bicyclic heterocyclic ring system, wherein said heterocyclic ring system is optionally substituted with one or more R⁵;
Y is C(R²)═C(R³) or S;
Z is N, C(H)—N(H)— or C(H)—N(CH₃)—
R¹ is —C(O)C(R^{A}) [symbol] (R^{B})ₚ, where
p is 1 or 2,
R^{A} is absent, hydrogen, deuterium, cyano, halogen, C1-C6 alkyl, halo-C1-C6 alkyl, heteroalkyl or hydroxy-C1-C6 alkyl, and
each R^{B} is independently hydrogen, deuterium, cyano, C1-C6 alkyl, alkoxy, halogen or halo-C1-C6 alkyl;
R² is absent, hydrogen, C1-C6 alkyl, alkoxy, halogen, cyano or C2-C6 alkynyl;
R³ is absent, hydrogen, C1-C6 alkyl, alkoxy, halogen, cyano or C2-C6 alkynyl;
R⁴ is 3-12 member heterocyclyl, 3-12 member cycloalkyl, C6-C14 aryl, C6-C14 aryl-C1-C6 alkyl or 5-14 member heteroaryl, wherein R⁴ is optionally substituted with one or more substituents independently selected from C1-C6 alkyl, C2-C6 alkenyl, C2-C6 alkynyl, 3-12 member cycloalkyl, amino, amino-C1-C6alkyl, hydroxy, alkoxy, halogen, cyano, and C1-C6 alkylamino; and
R⁵ is C1-C6 alkyl, cyano, C1-C6 alkyl-cyano, halogen, alkoxy, hydroxy, amino, C1-C6 alkylamino.

2. The compound or salt of claim 1, having the Formula (IA):

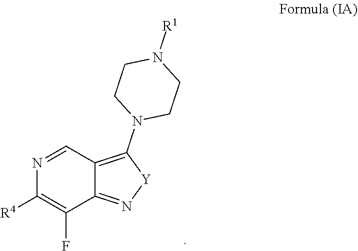

3. The compound or salt of claim 1, having the Formula:

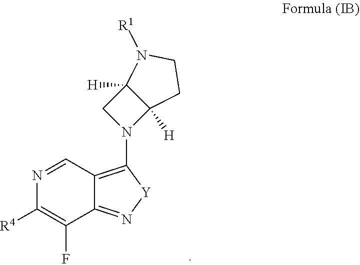

4. The compound or salt of claim 1, having the formula:

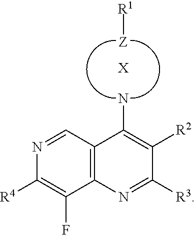

5. The compound or salt of claim 1, having the formula:

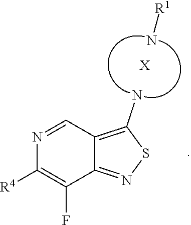

6. The compound or salt of claim 4, having the formula:

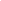

7. The compound or salt of claim 5, having the formula:

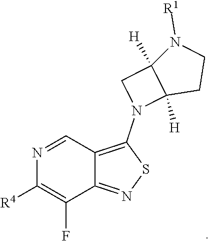

8. The compound or salt of claim 4, having the formula:

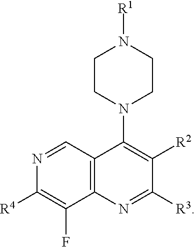

9. The compound or salt of claim 5, having the formula:

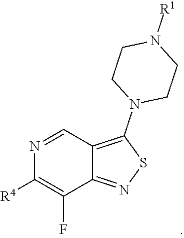

10. The compound or salt of claim 1, wherein X is a saturated bridged ring system optionally independently substituted with one or more C1-C6 alkyl or halogen.

11. The compound or salt of claim 1, wherein R¹—X is selected from the group consisting of: 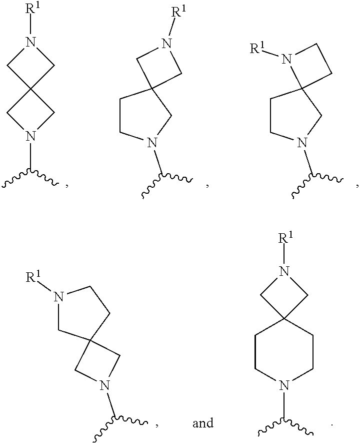
wherein R¹ is as defined for Formula (I).

12. The compound of claim 8, wherein R⁴ is an 8-chloro-7-fluoronaphth-1yl, 8-ethynyl-7-fluoronaphth-1yl or 8-ethynyl-7-hydroxynaphth-1yl substituent.

13. The compound of claim 10, wherein the saturated bicyclic ring system of claim 5 is optionally substituted with an alkyl, cyanoalkyl or halogen.

14. The compound of claim 1, wherein the compound is selected from: 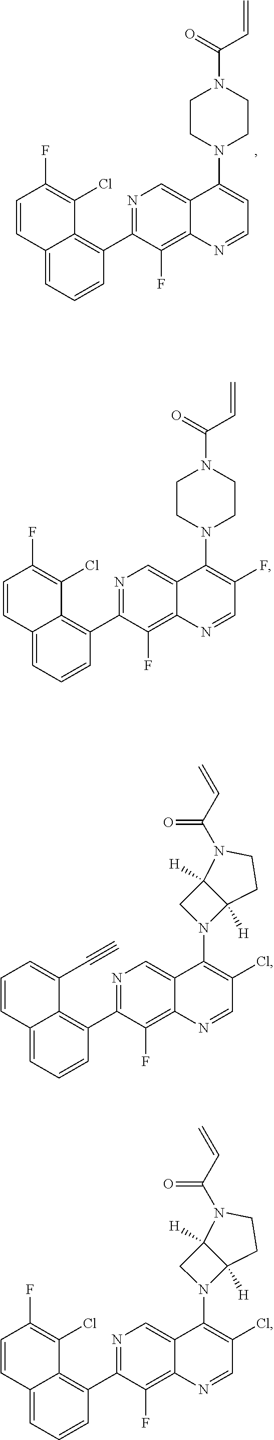 [SMILES: C=CC(=O)N1CCN(c2ccnc3c(F)c(-c4cccc5ccc(F)c(Cl)c45)ncc23)CC1 ; C=CC(=O)N1CCN(c2c(F)cnc3c(F)c(-c4cccc5ccc(F)c(Cl)c45)ncc23)CC1 ; C#Cc1cccc2cccc(-c3ncc4c(N5C[C@@H]6[C@H]5CCN6C(=O)C=C)c(Cl)cnc4c3F)c12 ; C=CC(=O)N1CC[C@@H]2[C@H]1CN2c1c(Cl)cnc2c(F)c(-c3cccc4ccc(F)c(Cl)c34)ncc12] 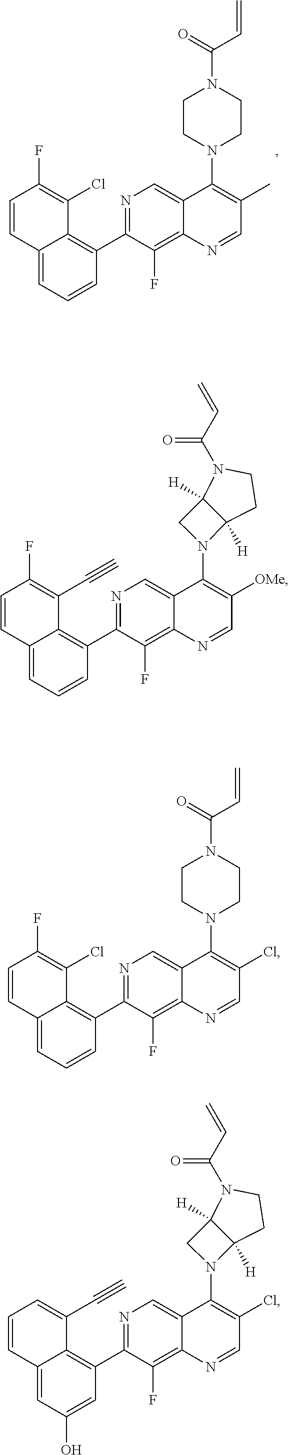 [SMILES: C=CC(=O)N1CCN(c2c(C)cnc3c(F)c(-c4cccc5ccc(F)c(Cl)c45)ncc23)CC1 ; C#Cc1c(F)ccc2cccc(-c3ncc4c(N5C[C@@H]6[C@H]5CCN6C(=O)C=C)c(OC)cnc4c3F)c12 ; C=CC(=O)N1CCN(c2c(Cl)cnc3c(F)c(-c4cccc5ccc(F)c(Cl)c45)ncc23)CC1 ; C#Cc1cccc2cc(O)cc(-c3ncc4c(N5C[C@@H]6[C@H]5CCN6C(=O)C=C)c(Cl)cnc4c3F)c12] 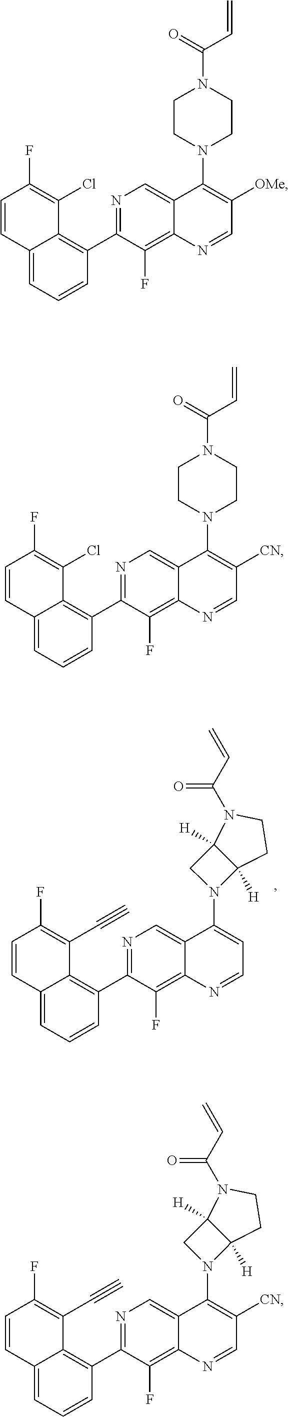 [SMILES: C=CC(=O)N1CCN(c2c(OC)cnc3c(F)c(-c4cccc5ccc(F)c(Cl)c45)ncc23)CC1 ; C=CC(=O)N1CCN(c2c(C#N)cnc3c(F)c(-c4cccc5ccc(F)c(Cl)c45)ncc23)CC1 ; C#Cc1c(F)ccc2cccc(-c3ncc4c(N5C[C@@H]6[C@H]5CCN6C(=O)C=C)ccnc4c3F)c12 ; C#Cc1c(F)ccc2cccc(-c3ncc4c(N5C[C@@H]6[C@H]5CCN6C(=O)C=C)c(C#N)cnc4c3F)c12] 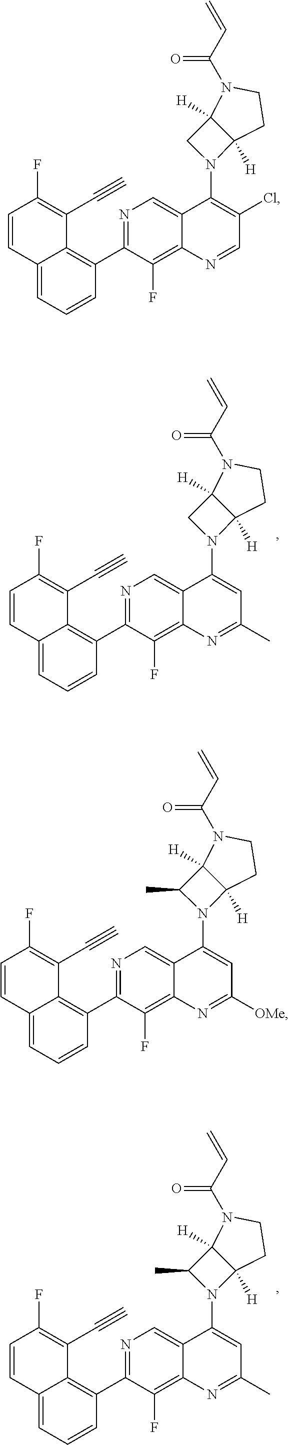 [SMILES: C#Cc1c(F)ccc2cccc(-c3ncc4c(N5C[C@@H]6[C@H]5CCN6C(=O)C=C)c(Cl)cnc4c3F)c12 ; C#Cc1c(F)ccc2cccc(-c3ncc4c(N5C[C@@H]6[C@H]5CCN6C(=O)C=C)cc(C)nc4c3F)c12 ; C#Cc1c(F)ccc2cccc(-c3ncc4c(N5[C@@H]6CCN(C(=O)C=C)[C@@H]6[C@@H]5C)cc(OC)nc4c3F)c12 ; C#Cc1c(F)ccc2cccc(-c3ncc4c(N5[C@@H]6CCN(C(=O)C=C)[C@@H]6[C@@H]5C)cc(C)nc4c3F)c12] 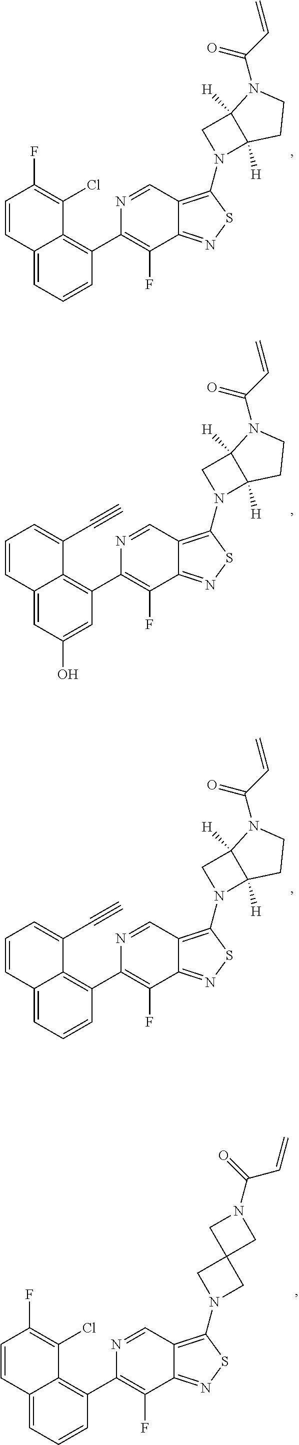 [SMILES: C=CC(=O)N1CC[C@@H]2[C@H]1CN2c1snc2c(F)c(-c3cccc4ccc(F)c(Cl)c34)ncc12 ; C#Cc1cccc2cc(O)cc(-c3ncc4c(N5C[C@@H]6[C@H]5CCN6C(=O)C=C)snc4c3F)c12 ; C#Cc1cccc2cccc(-c3ncc4c(N5C[C@@H]6[C@H]5CCN6C(=O)C=C)snc4c3F)c12 ; C=CC(=O)N1CC2(C1)CN(c1snc3c(F)c(-c4cccc5ccc(F)c(Cl)c45)ncc13)C2] 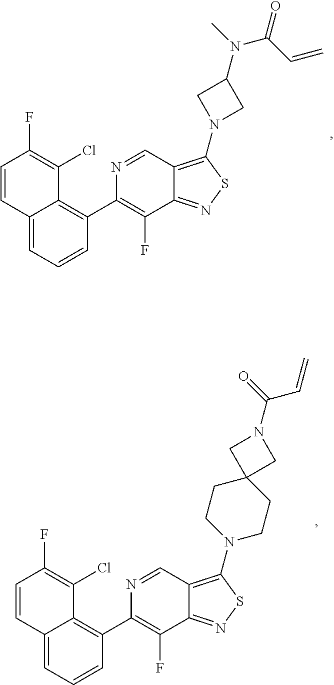 [SMILES: C=CC(=O)N(C)C1CN(c2snc3c(F)c(-c4cccc5ccc(F)c(Cl)c45)ncc23)C1 ; C=CC(=O)N1CC2(CCN(c3snc4c(F)c(-c5cccc6ccc(F)c(Cl)c56)ncc34)CC2)C1]
and pharmaceutically acceptable salts thereof.

15. A pharmaceutical composition, comprising a therapeutically effective amount of a compound of Formula (I) according to claim 1, and a pharmaceutically acceptable excipient.

16. A method for inhibiting KRas G12C activity in a cell, comprising contacting the cell in which inhibition of KRas G12C activity is desired with an effective amount of a compound of Formula (I) or a pharmaceutically acceptable salt thereof according to claim 1.

17. A method for treating cancer comprising administering to a patient having cancer a therapeutically effective amount of a compound of Formula (I) or a pharmaceutically acceptable salt thereof according to claim 1, alone or combined with a pharmaceutically acceptable carrier, excipient or diluents.

18. The method of claim 17, wherein the therapeutically effective amount of the compound is between about 0.01 to 100 mg/kg per day.

19. The method of claim 17, wherein the therapeutically effective amount of the compound is between about 0.1 to 50 mg/kg per day.

20. The method of claim 17, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

21. The method of claim 17, wherein the cancer is a KRas G12C-associated cancer.

22. The method of claim 17, wherein the cancer is non-small cell lung cancer.

23. A method for treating cancer in a patient in need thereof, the method comprising (a) determining that the cancer is associated with a KRas G12C mutation (e.g., a KRas G12C-associated cancer); and (b) administering to the patient a therapeutically effective amount of a compound of Formula (I) or a pharmaceutically acceptable salt thereof according to claim 1.
